SEQUENCE LISTING (38 sequences)

SEQ ID NO: 1 - DNA, 7854 bp, Dengue virus
  source 1..7854: mol_type = other DNA
gactcgggatgtgtaatcaactggaaaggcagagaactcaaatgtggaagcggcatttttgtcaccaatgaagtccacacctggacagagcaatataaattccaggccgactcccctaagagactatcagcggccattgggaaggcatgggaggagggtgtgtgtggaattcgatcagccactcgtctcgagaacatcatgtggaagcaaatatcaaatgaattaaaccacatcttacttgaaaatgacatgaaatttacagtggtcgtaggagacgttagtggaatcttggcccaaggaaagaaaatgattaggccacaacccatggaacacaaatactcgtggaaaagctggggaaaagccaaaatcataggagcagatgtacagaataccaccttcatcatcgacggcccaaacaccccagaatgccctgataaccaaagagcatggaacatttgggaagttgaagactatggatttggaattttcacgacaaacatatggttgaaattgcgtgactcctacactcaagtgtgtgaccaccggctaatgtcagctgccatcaaggatagcaaagcagtccatgctgacatggggtactggatagaaagtgaaaagaacgagacttggaagttggcaagagcctccttcatagaagttaagacatgcatctggccaaaatcccacactctatggagcaatggagtcctggaaagtgagatgataatcccaaagatatatggaggaccaatatctcagcacaactacagaccaggatatttcacacaaacagcagggccgtggcacttgggcaagttagaactagattttgatttatgtgaaggtaccactgttgttgtggatgaacattgtggaaatcgaggaccatctcttagaaccacaacagtcacaggaaagacaatccatgaatggtgctgtagatcttgcacgttaccccccctacgtttcaaaggagaagacgggtgctggtacggcatggaaatcagaccagtcaaggagaaggaagagaacctagttaagtcaatggtctctgcagggtcaggagaagtggacagtttttcactaggactgctatgcatatcaataatgatcgaagaggtaatgagatccagatggagcagaaaaatgctgatgactggaacattggctgtgttcctccttctcacaatgggacaattgacatggaatgatctgatcaggctatgtatcatggttggagccaacgcttcagacaagatggggatgggaacaacgtacctagctttgatggccactttcagaatgagaccaatgttcgcagtcgggctactgtttcgcagattaacatctagagaagttcttcttcttacagttggattgagtctggtggcatctgtagaactaccaaattccttagaggagctaggggatggacttgcaatgggcatcatgatgttgaaattactgactgattttcagtcacatcagctatgggctaccttgctgtctttaacatttgtcaaaacaactttttcattgcactatgcatggaagacaatggctatgatactgtcaattgtatctctcttccctttatgcctgtccacgacttctcaaaaaacaacatggcttccggtgttgctgggatctcttggatgcaaaccactaaccatgtttcttataacagaaaacaaaatctggggaaggaaaagctggcctctcaatgaaggaattatggctgttggaatagttagcattcttctaagttcacttctcaagaatgatgtgccactagctggcccactaatagctggaggcatgctaatagcatgttatgtcatatctggaagctcggccgatttatcactggagaaagcggctgaggtctcctgggaagaagaagcagaacactctggtgcctcacacaacatactagtggaggtccaagatgatggaaccatgaagataaaggatgaagagagagatgacacactcaccattctcctcaaagcaactctgctagcaatctcaggggtatacccaatgtcaataccggcgaccctctttgtgtggtatttttggcagaaaaagaaacagagatcaggagtgctatgggacacacccagccctccagaagtggaaagagcagtccttgatgatggcatttatagaattctccaaagaggattgttgggcaggtctcaagtaggagtaggagtttttcaagaaggcgtgttccacacaatgtggcacgtcaccaggggagctgtcctcatgtaccaagggaagagactggaaccaagttgggccagtgtcaaaaaagacttgatctcatatggaggaggttggaggtttcaaggatcctggaacgcgggagaagaagtgcaggtgattgctgttgaaccggggaagaaccccaaaaatgtacagacagcgccgggtaccttcaagacccctgaaggcgaagttggagccatagctctagactttaaacccggcacatctggatctcctatcgtgaacagagagggaaaaatagtaggtctttatggaaatggagtggtgacaacaagtggtacctacgtcagtgccatagctcaagctaaagcatcacaagaagggcctctaccagagattgaggacgaggtgtttaggaaaagaaacttaacaataatggacctacatccaggatcgggaaaaacaagaagataccttccagccatagtccgtgaggccataaaaagaaagctgcgcacgctagtcttagctcccacaagagttgtcgcttctgaaatggcagaggcgctcaagggaatgccaataaggtatcagacaacagcagtgaagagtgaacacacgggaaaggagatagttgaccttatgtgtcacgccactttcactatgcgtctcctgtctcctgtgagagttcccaattataatatgattatcatggatgaagcacattttaccgatccagccagcatagcagccagagggtatatctcaacccgagtgggtatgggtgaagcagctgcgattttcatgacagccactccccccggatcggtggaggcctttccacagagcaatgcagttatccaagatgaggaaagagacattcctgaaagatcatggaactcaggctatgactggatcactgatttcccaggtaaaacagtctggtttgttccaagcatcaaatcaggaaatgacattgccaactgtttaagaaagaatgggaaacgggtggtccaattgagcagaaaaacttttgacactgagtaccagaaaacaaaaaataacgactgggactatgttgtcacaacagacatatccgaaatgggagcaaacttccgagccgacagggtaatagacccgaggcggtgcctgaaaccggtaatactaaaagatggcccagagcgtgtcattctagccggaccgatgccagtgactgtggctagcgccgcccagaggagaggaagaattggaaggaaccaaaataaggaaggcgatcagtatatttacatgggacagcctctaaacaatgatgaggaccacgcccattggacagaagcaaaaatgctccttgacaacataaacacaccagaagggattatcccagccctctttgagccggagagagaaaagagtgcagcaatagacggggaatacagactacggggtgaagcgaggaaaacgttcgtggagctcatgagaagaggagatctacctgtctggctatcctacaaagttgcctcagaaggcttccagtactccgacagaaggtggtgctttgatggggaaaggaacaaccaggtgttggaggagaacatggacgtggagatctggacaaaagaaggagaaagaaagaaactacgaccccgctggctggatgccagaacatactctgacccactggctctgcgcgaattcaaagagttcgcagcaggaagaagaagcgtctcaggtgacctaatattagaaatagggaaacttccacaacatttaacgcaaagggcccagaacgccttggacaatctggttatgttgcacaactctgaacaaggaggaaaagcctatagacacgccatggaagaactaccagacaccatagaaacgttaatgctcctagctttgatagctgtgctgactggtggagtgacgttgttcttcctatcaggaaggggtctaggaaaaacatccattggcctactctgcgtgattgcctcaagtgcactgttatggatggccagtgtggaaccccattggatagcggcctctatcatactggagttctttctgatggtgttgcttattccagagccggacagacagcgcactccacaagacaaccagctagcatacgtggtgataggtctgttattcatgatattgacagtggcagccaatgagatgggattactggaaaccacaaagaaggacctggggattggtcatgcagctgctgaaaaccaccatcatgctgcaatgctggacgtagacctacatccagcttcagcctggactctctatgcagtggccacaacaattatcactcccatgatgagacacacaattgaaaacacaacggcaaatatttccctgacagctattgcaaaccaggcagctatattgatgggacttgacaagggatggccaatatcaaagatggacataggagttccacttctcgccttggggtgctattctcaggtgaacccgctgacgctgacagcggcggtattgatgctagtggctcattatgccataattggacccggactgcaagcaaaagctactagagaagctcaaaaaaggacagcagccggaataatgaaaaacccaactgtcgacgggatcgttgcaatagatttggaccctgtggtttacgatgcaaaatttgaaaaacagctaggccaaataatgttgttgatactttgcacatcacagatcctcctgatgcggaccacatgggccttgtgtgaatccatcacactagccactggacctctgactacgctttgggagggatctccaggaaaattctggaacaccacgatagcggtgtccatggcaaacatttttaggggaagttatctagcaggagcaggtctggccttttcattaatgaaatctctaggaggaggtaggagaggcacgggagcccaaggggaaacactgggagaaaaatggaaaagacagctaaaccaattgagcaagtcagaattcaacacttacaaaaggagtgggattatagaggtggatagatctgaagccaaagaggggttaaaaagaggagaaacgactaaacacgcagtgtcgagaggaacggccaaactgaggtggtttgtggagaggaaccttgtgaaaccagaagggaaagtcatagacctcggttgtggaagaggtggctggtcatattattgcgctgggctgaagaaagtcacagaagtgaaaggatacacgaaaggaggacctggacatgaggaaccaatcccaatggcaacctatggatggaacctagtaaagctatactccgggaaagatgtattctttacaccacctgagaaatgtgacaccctcttgtgtgatattggtgagtcctctccgaacccaactatagaagaaggaagaacgttacgtgttctaaagatggtggaaccatggctcagaggaaaccaattttgcataaaaattctaaatccctatatgccgagtgtggtagaaactttggagcaaatgcaaagaaaacatggaggaatgctagtgcgaaatccactctcaagaaactccactcatgaaatgtactgggtttcatgtggaacaggaaacattgtgtcagcagtaaacatgacatctagaatgctgctaaatcgattcacaatggctcacaggaagccaacatatgaaagagacgtggacttaggcgctggaacaagacatgtggcagtagaaccagaggtggccaacctagatatcattggccagaggatagagaatataaaaaatgaacacaaatcaacatggcattatgatgaggacaatccatacaaaacatgggcctatcatggatcatatgaggtcaagccatcaggatcagcctcatccatggtcaatggtgtggtgagactgctaaccaaaccatgggatgtcattcccatggtcacacaaatagccatgactgacaccacaccctttggacaacagagggtgtttaaagagaaagttgacacgcgtacaccaaaagcgaaacgaggcacagcacaaattatggaggtgacagccaggtggttatggggttttctctctagaaacaaaaaacccagaatctgcacaagagaggagttcacaagaaaagtcaggtcaaacgcagctattggagcagtgttcgttgatgaaaatcaatggaactcagcaaaagaggcagtggaagatgaacggttctgggaccttgtgcacagagagagggagcttcataaacaaggaaaatgtgccacgtgtgtctacaacatgatgggaaagagagagaaaaaattaggagagttcggaaaggcaaaaggaagtcgcgcaatatggtacatgtggttgggagcgcgctttttagagtttgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctattggccacgtcaatctttaagctaacctaccaaaacaaggtagtaagggtgcagagaccagcgaaaaatggaaccgtgatggatgtcatatccagacgtgaccagagaggaagtggacaggttggaacctatggcttaaacaccttcaccaacatggaggcccaactaataagacaaatggagtctgagggaatcttttcacccagcgaattggaaaccccaaatctagccgaaagagtcctcgactggttgaaaaaacatggcaccgagaggctgaaaagaatggcaatcagtggagatgactgtgtggtgaaaccaatcgatgacagatttgcaacagccttaacagctttgaatgacatgggaaaggtaagaaaagacataccgcaatgggaaccttcaaaaggatggaatgattggcaacaagtgcctttctgttcacaccatttccaccagctgattatgaaggatgggagggagatagtggtgccatgccgcaaccaagatgaacttgtaggtagggccagagtatcacaaggcgccggatggagcttgagagaaactgcatgcctaggcaagtcatatgcacaaatgtggcagctgatgtacttccacaggagagacttgagattagcggctaatgctatctgttcagccgttccagttgattgggtcccaaccagccgcaccacctggtcgatccatgcccaccatcaatggatgacaacagaagacatgttgtcagtgtggaatagggtttggatagaggaaaacccatggatggaggacaagactcatgtgtccagttgggaagacgttccatacctaggaaaaagggaagatcaatggtgtggttccctaataggcttaacagcacgagccacctgggccaccaacatacaagtggccataaaccaagtgagaaggctcattgggaatgagaattatctagacttcatgacatcaatgaagagattcaaaaacgagagtgatcccgaaggggcactctggtaa

SEQ ID NO: 2 - DNA, 7851 bp, Dengue virus
  source 1..7851: mol_type = other DNA
gatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagctcaagaagagggaatttgtggaatccgctcagtaacaagactggagaatctgatgtggaaacaaataacaccagaattgaatcacattctagcagaaaatgaggtgaagttaactatcatgacaggagacatcaaaggaatcatgcaggcaggaaaacgatctctgcggcctcagcccactgagctaaagtattcatggaaaacatggggcaaagcaaaaatgctctccacagagtctcataaccaaacctttctcattgatggccccgaaacagcagaatgccccaacacaaatagagcttggaattcgttggaagttgaagactacggctttggagtattcaccaccaatatatggctaaaattgaaagaaaaacaggatgcattctgcgactcaaaactcatgtcagcggccataaaagacaatagagccgtccatgccgatatgggttattggatagaaagtgcactcaatgacacatggaagatagagaaagcctctttcattgaagttaaaaattgccactggccaaaatcacacaccctctggagcaatggagtgctagaaagtgagatgataattccaaagaatctcgctggaccagtgtctcaacacaactatagaccaggctaccatacacaaatagcaggaccatggcatctaggtaagcttgagatggactttgatttctgcgatggaactacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaagagaaagaagaaaatttggttaactccttggtcacagccggacatgggcaggttgacaacttttcactaggagtcttgggaatggcattgttcctggaggaaatgcttaggacccgagtaggaacgaaacatgcaatactactagttgcagtttcttttgtgacattgatcacagggaacatgtcctttagagacctagggagagtggtggttatggtaggcgccactatgacggatgacataggtatgggcgtgacttatcttgccctactagcagccttcaaagtcagaccaacttttgcagctggactactcttgagaaagctgacctccaaggaattgatgatgactactataggaattgtactcctctcccaaagcaccataccagagaccattcttgaattgactgatgcgttagccttgggcatgatggtcctcaaaatggtaagaaatatggaaaagtatcaattagcagtgactatcatggctatcttgtgcgtcccaaacgcagtgatattacaaaacgcatggaaagtgagttgcacaatactagcagtggtgtccgtttccccattgcttttaacatcctcacagcaaaaaacggattggataccattagcattgacaatcaagggtctcaacccaacagctatttttctaacaaccctctcaagaaccagcaagaaaaggagctggccattaaatgaggctatcatggcagtcgggatggtgagcattttagccagttctctcctaaaaaatgatattcccatgacaggaccactagtggctggagggctcctcactgtgtgctacgtgctcactggacgatcggccgatttggaactggagagagcagccgatgtcaaatgggaagaccaggcagagatatcaggaagcagtccaatcctgtcaataacaatatcagaagatggtagcatgtcaataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaatcaagcaaaaagggattcttggatattcccagatcggagccggagtttacaaagaaggaacattccatacaatgtggcatgtcacacgtggcgctgtcctaatgcacaaaggaaagaggattgaaccatcatgggcggacgtcaagaaagatctaatatcatatggaggaggctggaagttagaaggagaatggaaggaaggagaagaagtccaggtattggcattggagcctggaaaaaatccaagagccgtccaaacaaaacctggtcttttcagaaccaacgccggaacaataggtgccgtatctctggacttttctcctggaacgtcaggatctccaatcattgacaaaaaaggaaaagttgtgggtctttatggtaatggtgttgttacaaggagtggagcatatgtgagtgctatagcccagacagaaaaaagcattgaagacaacccagagatcgaagatgacattttccgaaagagaagactgaccatactggacctccatccaggagcgggaaagacgaaaagataccttccggccatagtcagagaagctataaaacggggtttgagaacattaatcttggctcccactagagttgtggcagctgaaatggaggaagctcttagaggacttccaataagataccaaaccccagccatcagagctgagcacaccgggcgggagattgtggacctaatgtgtcatgccacatttaccatgaggctgctatcaccagttagagtgccaaactacaacctgattatcatggacgaagcccatttcacagacccagcaagtatagcagccagaggatacatctcaactcgagtggagatgggtgaggcagctgggattttcatgacagccactcccccgggaagcagagacccatttcctcagagcaatgcaccaatcatagatgaagaaagagaaatccctgaacgttcgtggaattctggacatgagtgggtcacggatttcaaagggaagactgtttggttcgttccaagtataaaagcaggaaatgatatagcagcttgcctgagaaaaaatggaaagaaagtgatacaactcagtaggaagacttttgattctgagtatgtcaagactagaaccaatgattgggatttcgtggttacaactgacatttcagaaatgggtgccaatttcaaggctgagagggttatagaccccagacgctgcatgaaaccagtcatactaacagatggtgaggagcgggtgattctggcaggacctatgccagtgacccactctagtgcagcacaaagaagagggagaataggaagaaatccaaaaaatgaaaatgaccagtacatatacatgggggaacctctggaaaatgatgaagactgtgcacactggaaagaagccaaaatgctcctagataacatcaacacaccagaaggaatcatccctagcatgttcgaaccagagcgtgaaaaagtggatgccattgatggcgaataccgcttgagaggagaagcaaggaaaacctttgtagacttaatgagaagaggagacctaccagtctggttggcctacaaagtggcagctgaaggcatcaactacgcagacagaaggtggtgttttgatggaatcaagaacaaccaaatcttggaagaaaacgtggaagttgaaatctggacaaaagaaggggaaaggaagaaattgaaacccagatggctggatgctaggatctattctgacccactggcgctaaaagaatttaaggaatttgcagccggaagaaagtctctgaccctgaacctaatcacagaaatgggtaggcttccaacctttatgactcagaaggcaagagacgcactggacaacttagcagtgctgcatacggctgaagcaggtggaagggcgtacaatcatgctctcagtgaactgccggagaccctggagacattgcttttactgacactgctggctacagtcacgggagggatctttttattcttgatgagcgggaggggcatagggaagatgaccctgggaatgtgctgcataatcacggctagcatcctcctatggtacgcacaaatacagccacactggatagcagcttcaataatactggagttttttctcatagttttgcttattccagaaccagaaaaacagagaacaccccaagacaaccaattgacctacgttgtcatagccatcctcacagtggtggccgcaaccatggcaaacgagatgggtttcctagaaaaaacgaagaaagatcttggattgggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtatccctaacagctatagccaaccaagccacagtgttaatgggtctcgggaaaggatggccattgtcaaagatggacatcggagttccccttctcgccattggatgctactcacaagtcaatcccataactctcacagcagctcttctcttattggtagcgcattatgccatcatagggccaggacttcaagcaaaagcaaccagggaagctcagaaaagagcagcggcgggcatcatgaaaaatccaaccgtcgatggaataacagtgattgacctagatccaataccctatgatccaaagtttgaaaagcagttgggacaagtaatgctcctagtcctctgcgtgactcaagtattgatgatgaggactacatgggctctgtgtgaggctttaaccttagctaccgggcccatctccacactgtgggaaggaaatccaggaagattttggaacactaccattgcggtgtcaatggctaacatttttagagggagttacttggctggagctggacttctcttttctattatgaagaacacaaccaacacaagaaggggaactggcaacataggagagacgcttggagagaaatggaaaagccgattgaacgcactgggaaaaagtgaattccagatctacaagaaaagtggaatccaggaagtggatagaaccttagcaaaagaaggcatcaaaagaggagaaacggaccatcacgctgtgtcgcgaggttcagcaaaactgagatggttcgttgagagaaacatggtcacaccagaagggaaagtagtggacctcggttgtggcagaggaggctggtcatactattgtggaggactaaaaaacgtaagagaagtcaaaggcctaacaaaaggaggaccaggacacgaagaacccatccccatgtcaacatatgggtggaatctagtgcgtcttcaaagtggagtcgacgttttcttcatcccgccagaaaagtgtgacacattgttgtgtgacataggggagtcatcaccaaatcccacagtggaagcaggacgaacactcagagtccttaatttagtagaaaattggttgaacaacaacactcaattttgcataaaagttctcaacccatatatgccctcagtcatagaaaaaatggaaacactgcaaaggaaatatggaggagccttagtgaggaatccactctcacgaaactccacacatgagatgtactgggtatctaatgcttccgggaacatagtgtcatcagtgaacatgatttcaaggatgttgatcaacagattcacaatgagacacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtggtcaggctgctgacaaaaccttgggacgtcgtccccatggtgacacagatggcaatgacagacacgactccatttggacaacagcgtgtttttaaagaaaaagtggacacgagaacccaagaaccgaaagaaggcacgaagaaactaatgaaaatcacagcagagtggctgtggaaagaattagggaagaaaaagacacctaggatgtgcaccagagaagaattcacaagaaaggtgagaagcaatgcagccttgggggccatattcactgatgagaacaagtggaagtcggcacgtgaggctgttgaagacagtaggttttgggagctggttgacaaggaaaggaatctccatcttgaaggaaagtgtgaaacatgtgtgtacaacatgatgggaaaaagagagaagaagctaggggaattcggcaaggcaaaaggtagcagagccatatggtacatgtggcttggagctcgcttcctagagtttgaagccctaggattcctaaatgaagatcactggttctccagagagaactccctgagtggagtggaaggagaagggctgcacaagctaggttacattctaagagacgtgagcaagaaagagggaggagctatgtatgccgatgacacagcaggatgggacacaagaatcacactagaagacctaaaaaatgaagaaatggtaacaaaccacatggaaggagaacacaagaaactagccgaggccattttcaaactaacgtaccaaaacaaggtggtgcgtgtgcaaagaccaacaccaagaggcacagtaatggacattatatcgagaagagaccaaagaggtagtggacaagttggtacctatggactcaatactttcaccaatatggaagcccaactaatcagacagatggagggagaaggagtcttcaaaaacattcagcacctgacagtcacagaagaaatcgccgtgcaaaactggttggcaagagtggggcgcgaaaggttatcaagaatggccatcagtggagatgattgtgttgtgaaacctttagatgacaggttcgcaagcgctttaacagctctaaatgacatgggaaagattaggaaagacatacaacaatgggaaccttcaagaggatggaacgattggacacaagtgcccttctgttcacaccatttccatgagctaatcatgaaagatggtcgcgtacttgttgttccatgtagaaaccaagatgaactgattggcagagcccgaatctcccaaggagcagggtggtctttgcgggagacggcctgtttggggaagtcctacgcccaaatgtggagcttgatgtacttccacagacgcgacctcaggctggcggcaaatgccatttgctcggcagtaccatcacattgggttccaacaagtcgaacaacctggtccatacacgccaaacatgaatggatgacaacggaagacatgctgacagtctggaacagggtgtggattcaagaaaacccatggatggaagacaaaaccccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaggaatacacagattacatgccatccatgaaaagatttagaagagaagaggaagaagcaggagtcctgtggtag

SEQ ID NO: 3 - DNA, 7855 bp, Dengue virus
  source 1..7855: mol_type = other DNA
gacatgggatgtgttataaactggaaaggcaaagaactcaaatgtggaagtggaatcttcgtcaccaatgaggtccatacctggacagagcaatacaaattccaagcagactcaccaaaaagattggcgacagccattgcaggcgcttgggagaatggagtgtgcggaattaggtcaacaaccagaatggagaatctcctgtggaagcaaatagccaatgaactgaactacatattgtgggaaaacaatatcaaattaacggtagttgtgggcgatataattggggtcttagagcaaggaaaaagaacactaacaccacaacccatggagctaaaatactcatggaaaacgtggggaaaggcaaaaatagtgacagctgaaatacaaaattcctctttcataatagacgggccaaacacaccggagtgtccaagtgcctcaagagcatggaatgtgtgggaggtggaagattacgggttcggagtcttcacaaccaacatatggctgaaactccgagaggtgtacacccaactatgtgaccataggctaatgtcggcagctgtcaaggatgagagggccgtgcatgccgacatgggctactggatagaaagccaaaagaatggaagttggaagctagaaaaagcatccctcatagaggtgaaaacctgcacatggccaaaatcacataccctttggagtaatggtgtgctagagagtgacatgatcatcccaaagagtctagctggtcctatttcgcaacacaactacaggcccgggtaccacacccagacggcgggaccctggcatttaggaaaattagacctggacttcaactattgcgaaggaacaacagttgtcatcacagaaaactgtgggacaagaggcccatcattgagaacaacaacagtgtcagggaagttaatacacgaatggtgttgccgttcgtgcacacttcctcccttgcgatacatgggagaagacggttgctggtatggcatggaaatcagacccatcagtgagaaagaagagaacatggtaaagtctttagtctcagcgggaagtggaaaggtggacaactttacaatgggtgtcttgtgtttggcaatcctctttgaagaggtgatgagaggaaaatttgggaagaaacacatgattgcgggggttttcttcacgtttgtgctccttctctcagggcaaataacatggagagacatggcgcacacactaataatgattgggtccaacgcatctgacaagatgggaatgggcgttacctacctagctttaattgcaacatttaaaatccagccattcttggctttgggatttttcctaagaaaactgacatccagagaaaatttattgttaggagttgggctggctatggcaacaacgttacaactgccagaggacattgaacaaatggcaaatggaatcgctctggggctcatggcgcttaaactgataacacaatttgaaacataccaattatggacagcattaatctccttaacgtgttcaaatacaatgtttacgttgactgttgcctggagaacagccaccctgattttggccggagtttcgcttttaccagtgtgtcagtcttcgagcatgaggaaaacagactggcttccaatggcagtggcagctatgggagttccacctctaccactttttatttttagcttgaaagacacacttaaaaggagaagctggccactgaatgaaggggtgatggctgttgggcttgtgagcattctggccagttctctccttagaaatgatgtacccatggctggaccattagtggccgggggcttgctgatagcgtgctacgtcataactggcacgtcagcagacctcaccgtagaaaaagcagcagatataacatgggaggaagaggctgagcaaacaggagtgtcccacaacttaatgatcacagttgatgatgatggaacaatgagaataaaagatgatgagactgagaatatcctaacagtgcttttgaaaacagcattactaatagtatcaggagtctttccatactccatacccgcaacattgctggtctggcacacttggcaaaagcaaacccaaagatccggcgttctatgggacgtacccagccccccagagacacagaaagcagaactggaagagggggtctataggatcaaacagcaaggaatttttgggaaaacccaagtaggggttggagtacagaaagaaggagtcttccacaccatgtggcacgttacaagaggggcggtgttgacatataatgggaaaagactggaaccaaactgggctagtgtgaaaaaagatctgatttcatacggaggaggatggagattgagcgcacaatggcaaaagggggaggaggtgcaggttattgccgtagagcctgggaagaacccaaagaactttcaaaccatgccaggcacttttcagactacaacaggggaaataggagcaattgcactggatttcaagcctggaacttcaggatctcctatcataaacagagagggaaaggtagtgggactgtatggcaatggagtggttacaaagaatggtggctacgtcagcggaatagcgcaaacgaatgcagaaccagatggaccgacaccagaattggaagaagagatgttcaaaaagcgaaatctaaccataatggatcttcatcctgggtcaggaaagacacggaaataccttccagctattgttagagaggcaatcaagagacgtctaaggactctaattttggcaccgacaagggtggttgcagctgagatggaagaagcattgaaagggctcccaataaggtaccaaacaacagcaacaaaatctgaacacacaggaagagagattgttgatctaatgtgccacgcaacgttcacaatgcgcctgctgtcaccagttagggttccaaattataacttgataataatggatgaagcccatttcacagacccagccagtatagcggctagagggtacatatcgactcgtgttggaatgggagaggcagccgcaattttcatgacagcaacgccccctggaacagctgatgcctttcctcagagcaacgctccaattcaagatgaagaaagggacataccagaacgctcatggaattcaggcaatgaatggataaccgacttcgctgggaaaacggtgtggtttgtccccagcattaaagccggaaatgacatagcaaactgcttgcgaaaaaacgggaaaaaggtcattcaacttagtaggaagacttttgacacagaatatcagaaaactaaactgaatgattgggacttcgtagtgacaactgacatttcagaaatgggggccaatttcaaagcagatagagtgatcgacccaagaagatgtctcaaaccagtgatcctgacagatggaccagagcgggtgatcctggctggaccaatgccagtcaccgcggcgagtgctgcgcaaaggagagggagagttggcaggaacccacaaaaagaaaatgaccagtacatattcacgggccaacctctcaataatgatgaagaccacgctcactggacagaagcaaaaatgctgctggacaacattaatacaccagaagggatcataccagctctctttgagccagaaagggagaagtcagccgccatagacggtgagtatcgcttgaaaggtgagtctaggaagactttcgtggaactcatgaggaggggtgaccttccagtctggttagcccataaagtagcatcagaagggatcaaatatacagatagaaaatggtgctttgatggacaacgtaataatcaaattttagaggagaacatggatgtggaaatctggacaaaggaaggagaaaagaaaaaattgagacctaggtggcttgatgcccgcacttattcagatcccttagcactcaaggaattcaaggactttgcggctggcagaaagtcaatcgcccttgatcttgtgacagaaataggaagagtgccttcacatctagcccatagaacgagaaacgctctggacaatctggtgatgctgcatacgtcagaacatggcggtagggcctacaggcatgcggtggaggaactaccagagacaatggaaacactcctactcttgggactcatgatcttgttgacaggtggagcaatgcttttcttaatatcaggtaaagggattggaaagacctcaataggactcatttgtgtaattgcttccagcggcatgttgtggatggccgaaatcccactccaatggatcgcgtcggctatagtcctggagttttttatgatggtgttgcttataccagaaccagaaaagcagagaactccccaagacaaccaactcgcatatgtcgtgataggcatactcacattggctgcaataatagcagccaatgaaatgggattgttggaaactacaaagagagatttaggaatgtctaaggagccaggtgttgtctctccaaccagctatttagatgtggacttgcacccagcatcagcctggacattgtacgccgtggccactacagtaataacaccaatgttaagacataccatagagaattctacagcaaatgtgtctctggcagctatagccaaccaggcagtggtcctgatgggtttggacaaaggatggccaatatcaaaaatggacttaggagtacccctactggcattgggttgctattcacaagtgaacccactgactctaacagcggcagtactcttgctgatcacacattatgctattataggtccaggattgcaggcaaaagccactcgtgaagctcaaaaaaggacagctgctggaataatgaagaatccaacggtggatgggataatgacaatagacctagatcccgtaatatatgattcaaaatttgaaaaacaactgggacaggttatgctcctggttttgtgtgcagttcaacttttgttaatgagaacatcatgggccttgtgtgaagctttaactctagctacaggaccaataacaacactctgggaaggatcacctgggaagttttggaacaccacgatagctgtttccatggcgaacatttttagagggagctatttagcaggagctgggcttgctttttctataatgaaatcagttggaacaggaaaaagaggaacaggctcacaaggtgaaactttaggagaaaaatggaaaaagaaattaaatcaattatcccggaaagagtttgacctttacaagaaatctggaatcactgaagtggatagaacagaagccaaagaagggttgaaaagaggagaaacaacacatcatgccgtgtccagaggtaccgcgaaacttcaatggtttgtggagagaaacatggtcattcccgaaggaagagtcatagacttgggctgtggaagaggaggctggtcatattactgtgcaggactgaaaaaagtcacagaagtgcgaggatacacaaaaggcggtccaggacgcgaagaaccagtacctatgtccacatatggatggaacatagttaagttaatgagtggaaaggatgtgttttatcttccacctgaaaagtgtgacaccctgttgtgtgacattggagaatcttcaccaagcccaacagtggaagaaagcagaactataagagttttgaagatggttgaaccatggctaaagaacaaccaattttgcattaaagtattgaacccttacatgccaactgtgattgagcacctagaaagattacaaaggaaacatggaggaatgcttgtgagaaatccactttcacgaaactccacgcacgaaatgtactggatatctaatggcacaggtaacattgtcgcttcagtcaatatggtatctagactgctactgaacaggttcacgatgacacacagaagacccaccattgagaaagatgtggatttaggagcaggaactcgacatgttaatgcggaaccagaaacacccaacatggatgtcattggggaaagaataaaaaggatcaaggaagagcataattcaacatggcactatgatgacgaaaacccctacaaaacgtgggcttaccatggatcttatgaagttaaagccacaggctcagcctcctccatgataaatggagtcgtgaaactcctcactaaaccatgggatgtggtgcccatggtgacacagatggcaatgacagatacaactccatttggccagcagagagtctttaaagagaaagtggacaccaggacacccaggtccatgccaggaacaagaagggtcatggggatcacagcggagtggctctggagaaccttgggaaggaacaaaaaacccaggctatgcacaagggaagagtttacaaaaaaggtcagaaccaacgcagccatgggcgccgttttcacagaggagaaccaatgggatagcgcgaaagctgctgttgaggatgaggatttttggaaacttgtggacagagaacgtgaactccacaaattgggcaagtgtggaagctgtgtttacaacatgatgggcaagagagagaagaaacttggagagtttggcaaagcaaaaggcagtagagctatatggtacatgtggttgggagccaggtaccttgagttcgaagcccttggattcttaaatgaagaccactggttctcgcgtgagaactcttacagtggagtggaaggagaaggactgcacaagctaggctatatattaagggacatttccaagatacccggaggagctatgtatgctgatgacacagctggttgggacacaagaataacagaagatgacctgcacaatgaggaaaagatcacacagcaaatggaccctgaacacaggcagttagcgaacgctatatttaagctcacataccaaaacaaagtggtcaaagttcaacgaccgactccaacaggcacggtaatggacatcatatctaggaaagaccaaagaggcagtggacaggtaggaacttatggtctgaatacattcaccaacatggaagcccagttaatcagacaaatggaaggagaaggtgtgctgtcaaaggcagacctcgagaaccctcatctgccagagaagaaaattacacaatggttggaaaccaaaggagtggagaggttaagaagaatggccattagcggggatgattgcgtagtgaaaccaatcgatgacaggttcgctaatgccctgcttgctctgaacgacatgggaaaggttcggaaagacatacctcaatggcagccatcaaagggatggcatgattggcaacaggttcctttctgctcccaccacttttcatgaattgatcatgaaagatggaagaaagttggtggttccctgcagaccccaggacgaactaataggaagagcaagaatctctcaaggagcgggatggagccttagagaaaccgcatgtctggggaaagcctacgctcaaatgtggagtctcatgtattttcacagaagagacctcagactagcatccaacgccatatgttcagcagtaccagtccactgggtccccacaagtagaacgacatggtctattcatgctcaccatcagtggatgaccacagaagacatgcttactgtctggaacagggtatggatcgaggacaatccatggatggaagacaaaactccagtcacaacctgggaaaatgttccatatttagggaagagagaagaccaatggtgcggatcacttattggtctcacttccagagcaacctgggcccagaacatacccacagcaattcaacaggtgagaagccttataggcaatgaagagtttctggactacatgccttcaatgaagagatttaggaaggaggaggagtcggagggagccatttggtaa

SEQ ID NO: 4 - DNA, 7842 bp, Dengue virus
  source 1..7842: mol_type = other DNA
gacatgggttgtgtggcgtcatggagtgggaaagaattgaagtgtggaagcggaatttttgtggttgacaacgtgcacacttggacagaacagtacaaatttcaaccagagtccccagcgagactagcgtctgcaatattaaatgcccacaaagatggggtctgtggaattagatcaaccacgaggctggaaaatgtcatgtggaagcaaataaccaacgagctaaactatgttctctgggaaggaggacatgacctcactgtagtggctggggatgtgaagggggtgttgaccaaaggcaagagagcactcacacccccagtgagtgatctgaaatattcatggaagacatggggaaaagcaaaaatcttcaccccagaagcaagaaatagcacatttttaatagacggaccagacacctctgaatgccccaatgaacgaagagcatggaactctcttgaggtggaagactatggatttggcatgttcacgaccaacatatggatgaaattccgagaaggaagttcagaagtgtgtgaccacaggttaatgtcagctgcaattaaagatcagaaagctgtgcatgctgacatgggttattggatagagagctcaaaaaaccagacctggcagatagagaaagcatctcttattgaagtgaaaacatgtctgtggcccaagacccacacactgtggagcaatggagtgctggaaagccagatgctcattccaaaatcatatgcgggccctttttcacagcacaattaccgccagggctatgccacgcaaaccgtgggcccatggcacttaggcaaattagagatagactttggagaatgccccggaacaacagtcacaattcaggaggattgtgaccatagaggcccatctttgaggaccaccactgcatctggaaaactagtcacgcaatggtgctgccgctcctgcacgatgcctcccttaaggttcttgggagaagatgggtgctggtatgggatggagattaggcccttgagtgaaaaagaagagaacatggtcaaatcacaggtgacggccggacagggcacatcagaaactttttctatgggtctgttgtgcctgaccttgtttgtggaagaatgcttgaggagaagagtcactaggaaacacatgatattagttgtggtgatcactctttgtgctatcatcctgggaggcctcacatggatggacttactacgagccctcatcatgttgggggacactatgtctggtagaataggaggacagatccacctagccatcatggcagtgttcaagatgtcaccaggatacgtgctgggtgtgtttttaaggaaactcacttcaagagagacagcactaatggtaataggaatggccatgacaacggtgctttcaattccacatgaccttatggaactcattgatggaatatcactgggactaattttgctaaaaatagtaacacagtttgacaacacccaagtgggaaccttagctctttccttgactttcataagatcaacaatgccattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgagggcataatggctgtgggtttggttagtctcttaggaagcgctcttttaaagaatgatgtccctttagctggcccaatggtggcaggaggcttacttctggcggcttacgtgatgagtggtagctcagcagatctgtcactagagaaggccgccaacgtgcagtgggatgaaatggcagacataacaggctcaagcccaatcgtagaagtgaagcaggatgaagatggctctttctccatacgggacgtcgaggaaaccaatatgataacccttttggtgaaactggcactgataacagtgtcaggtctctaccccttggcaattccagtcacaatgaccttatggtacatgtggcaagtgaaaacacaaagatcaggagccctgtgggacgtcccctcacccgctgccactaaaaaagccgcactgtctgaaggagtgtacaggatcatgcaaagagggttattcgggaaaactcaggttggagtagggatacacatggaaggtgtatttcacacaatgtggcatgtaacaagaggatcagtgatctgccacgagactgggagattggagccatcttgggctgacgtcaggaatgacatgatatcatacggtgggggatggaggcttggagacaaatgggacaaagaagaagacgttcaggtcctcgccatagaaccaggaaaaaatcctaaacatgtccaaacgaaacctggccttttcaagaccctaactggagaaattggagcagtaacattagatttcaaacccggaacgtctggttctcccatcatcaacaggaaaggaaaagtcatcggactctatggaaatggagtagttaccaaatcaggtgattacgtcagtgccataacgcaagccgaaagaattggagagccagattatgaagtggatgaggacatttttcgaaagaaaagattaactataatggacttacaccccggagctggaaagacaaaaagaattcttccatcaatagtgagagaagccttaaaaaggaggctacgaactttgattttagctcccacgagagtggtggcggccgagatggaagaggccctacgtggactgccaatccgttatcagaccccagctgtgaaatcagaacacacaggaagagagattgtagacctcatgtgtcatgcaaccttcacaacaagacttttgtcatcaaccagggttccaaattacaaccttatagtgatggatgaagcacatttcaccgatccttctagtgtcgcggctagaggatacatctcgaccagggtggaaatgggagaggcagcagccatcttcatgaccgcaacccctcccggagcgacagatccctttccccagagcaacagcccaatagaagacatcgagagggaaattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagagtatccaaaaacgaaactcacggactgggactttgtggtcactacagacatatctgaaatgggggccaattttagagccgggagagtgatagaccctagaagatgcctcaagccagttatcctaccagatgggccagagagagtcattttagcaggtcctattccagtgactccagcaagcgctgctcagagaagagggcgaataggaaggaacccagcacaagaagacgaccaatacgttttctccggagacccactaaaaaatgatgaagatcatgcccactggacagaagcaaagatgctgcttgacaatatctacaccccagaagggatcattccaacattgtttggtccggaaagggaaaaaacccaagccattgatggagagtttcgcctcagaggggaacaaaggaagacttttgtggaattaatgaggagaggagaccttccggtgtggctgagctataaggtagcttctgctggcatttcttacgaagatcgggaatggtgcttcacaggggaaagaaataaccaaattttagaagaaaacatggaggttgaaatttggactagagagggagaaaagaaaaagctaaggccaagatggttagatgcacgtgtatacgctgaccccatggctttgaaggatttcaaggagtttgccagtggaaggaagagtataactctcgacatcctaacagagattgccagtttgccaacttacctttcctctagggccaagctcgcccttgataacatagtcatgctccacacaacagaaagaggagggagggcctatcaacacgccctgaacgaacttccggagtcactggaaacactcatgcttgtagctttactaggtgctatgacagcaggcatcttcctgtttttcatgcaagggaaaggaatagggaaattgtcaatgggtttgataaccattgcggtggctagtggcttgctctgggtagcagaaattcaaccccagtggatagcggcctcaatcatactagagttttttctcatggtactgttgataccggaaccagaaaaacaaaggaccccacaagacaatcaattgatctacgtcatattgaccattctcaccatcattggtctaatagcagccaacgagatggggctgattgaaaaaacaaaaacggattttgggttttaccaggtaaaaacagaaaccaccatcctcgatgtggacttgagaccagcttcagcatggacgctctatgcagtagccaccacaattctgactcccatgctgagacacaccatagaaaacacgtcggccaacctatctctagcagccattgccaaccaggcagccgtcctaatggggcttggaaaaggatggccgctccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctgggatcatgaaaaatcccacagtggacgggataacagtaatagatctagaaccaatatcctatgacccaaaatttgaaaagcaattagggcaggtcatgctactagtcttgtgtgctggacaactactcttgatgagaacaacatgggctttctgtgaagtcttgactttggccacaggaccaatcttgaccttgtgggagggcaacccgggaaggttttggaacacgaccatagccgtatccaccgccaacattttcaggggaagttacttggcgggagctggactggctttttcactcataaagaatgcacaaacccctaggaggggaactgggaccacaggagagacactgggagagaagtggaagagacagctaaactcattagacagaaaagagtttgaagagtataaaagaagtggaatactagaagtggacaggactgaagccaagtctgccctgaaagatgggtctaaaatcaagcatgcagtatcaagagggtccagtaagatcagatggattgttgagagagggatggtaaagccaaaagggaaagttgtagatcttggctgtgggagaggaggatggtcttattacatggcgacactcaagaacgtgactgaagtgaaagggtatacaaaaggaggtccaggacatgaagaaccgattcccatggctacttatggttggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtccttaacccctacatgccaacagtcatagaagagctggagaaactgcagagaaaacatggtgggaaccttgtcagatgcccgctgtccaggaactccacccatgagatgtattgggtgtcaggagcgtcgggaaacattgtgagctctgtgaacacaacatcaaagatgttgttgaacaggttcacaacaaggcataggaaacccacttatgagaaggacgtagatcttggggcaggaacgagaagtgtctccactgaaacagaaaaaccagacatgacaatcattgggagaaggcttcagcgattgcaagaagagcacaaagaaacctggcattatgatcaggaaaacccatacagaacctgggcgtatcatggaagctatgaagctccttcgacaggctctgcatcctccatggtgaacggggtggtaaaactgctaacaaaaccctgggatgtgattccaatggtgactcagttagccatgacagatacaaccccttttgggcaacaaagagtgttcaaagagaaggtggataccagaacaccacaaccaaaacccggtacacgaatggttatgaccacgacagccaattggctgtgggccctccttggaaagaagaaaaatcccagactgtgcacaagggaagagttcatctcaaaagttagatcaaacgcagccataggcgcagtctttcaggaagaacagggatggacatcagccagtgaagctgtgaatgacagccggttttgggaactggttgacaaagaaagggccctacaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatatatcctggaggagatagacaagaaggatggagacctaatgtatgctgatgacacagcaggctgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgcggggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgaaagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccaccactttcacaagatctttatgaaggatggccgctcactagttgttccatgtagaaaccaggatgaactgatagggagagccagaatctcgcagggagctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccacgctcatcaccagtggatgaccactgaagatatgctcaaagtgtggaacagagtgtggatagaagacaaccctaatatgactgacaagactccagtccattcgtgggaagatataccttacctagggaaaagagaggatttgtggtgtggatccctgattggactttcttccagagccacctgggcgaagaacattcatacggccataacccaggtcaggaacctgatcggaaaagaggaatacgtggattacatgccagtaatgaaaagatacagtgctccttcagagagtgaaggagttctgtaa

SEQ ID NO: 5 - DNA, 90 bp, Dengue virus
  source 1..90: mol_type = other DNA
agttgttagtctacgtggaccgacaagaacagtttcgaatcggaagcttgcttaacgtagttctaacagttttttattagagagcagcat

SEQ ID NO: 6 - DNA, 96 bp, Dengue virus
  source 1..96: mol_type = other DNA
agttgttagtctacgtggaccgacaaagacagattctttgagggagctaagctcaacgtagttctaacagttttttaattagagagcagatctctg

SEQ ID NO: 7 - DNA, 58 bp, Dengue virus
  source 1..58: mol_type = other DNA
gactcggaagcttgcttaacgtagtgctaacagttttttgttagagagcagatctctg

SEQ ID NO: 8 - DNA, 101 bp, Dengue virus
  source 1..101: mol_type = other DNA
agttgttagtctgtgtggaccgacaaggacagttccaaatcggaagcttgcttaacacagttctaacagtttgtttgaatagagagcagatctctggaaaa

SEQ ID NO: 9 - DNA, 462 bp, Dengue virus
  source 1..462: mol_type = other DNA
gccaactcattcacaaaataaaggaaaataaaaaatcaaacaaggcaagaagtcaggccggattaagccatagcacggtaagagctatgctgcctgtgagccccgtccaaggacgtaaaatgaagtcaggccgaaagccacggttcgagcaagccgtgctgcctgtagctccatcgtggggatgtaaaaacccgggaggctgcaaaccatggaagctgtacgcatggggtagcagactagtggttagaggagacccctcccaagacacaacgcagcagcggggcccaacaccaggggaagctgtaccctggtggtaaggactagaggttagaggagaccccccgcacaacaacaaacagcatattgacgctgggagagaccagagatcctgctgtctctacagcatcattccaggcacagaacgccaaaaaatggaatggtgctgttgaatcaacaggttct

SEQ ID NO: 10 - DNA, 451 bp, Dengue virus
  source 1..451: mol_type = other DNA
aaagcagaactaatttaaaacaaggctggaagtcaggtcggattaagccatagtacggaaaaaactatgctacctgtgagccccgtccaaggacgttaaaagaagtcaggccatcataaatgccatagcttgagtaaactatgcagcctgtagctccacctgagaaggtgtaaaaaatccgggaggccacaaaccatggaagctgtacgcatggcgtagtggactagcggttagaggagacccctcccttacaaatcgcagcaacaatgggggcccaaggcgagatgaagctgtagtctcgctggaaggactagaggttagaggagacccccccgaaacaaaaaacagcatattgacgctgggaaagaccagagatcctgctgtctcctcagcatcattccaggcacagaacgccagaaaatggaatggtgctgttgaatcaacaggttct

SEQ ID NO: 11 - DNA, 440 bp, Dengue virus
  source 1..440: mol_type = other DNA
acgtaggaagtgaaaaagaggctaactgtcaggccacattaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaataaaaacagcatattgacgctgggagagaccagagatcctgctgtctcctcagcatcattccaggcacagaacgccagaaaatggaatggtgctgttgaatcaacaggttct

SEQ ID NO: 12 - DNA, 384 bp, Dengue virus
  source 1..384: mol_type = other DNA
ttaccaacaacaaacaccaaaggctattgaagtcaggccacttgtgccacggtttgagcaaaccgtgctgcctgtagctccgccaataatgggaggcgtaataatccccagggaggccatgcgccacggaagctgtacgcgtggcatattggactagcggttagaggagacccctcccatcactgataaaacgcagcaaaagggggcccgaagccaggaggaagctgtactcctggtggaaggactagaggttagaggagacccccccaacacaaaaacagcatattgacgctgggaaagaccagagatcctgctgtctctgcaacatcaatccaggcacagagcgccgcaagatggattggtgttgttgatccaacaggttct

SEQ ID NO: 13 - DNA, 102 bp, Dengue virus
  source 1..102: mol_type = other DNA
atgaacaaccaacggaaaaagacgggtcgaccgtctttcaatatgctgaaacgcgcgagaaaccgcgtgtcaactgtttcacagttggcgaagagattctca

SEQ ID NO: 14 - DNA, 102 bp, Dengue virus
  source 1..102: mol_type = other DNA
atgaataaccaacggaaaaaggcgagaaacacgcctttcaatatgctgaaacgcgagagaaaccgcgtgtcgactgtgcaacagctgacaaagagattctca

SEQ ID NO: 15 - DNA, 102 bp, Dengue virus
  source 1..102: mol_type = other DNA
atgaacaaccaacggaagaaggcgggaaaaccgtctatcaatatgctgaaacgcgtgagaaaccgtgtgtcaactggatcacagttggcgaagagattctca

SEQ ID NO: 16 - DNA, 99 bp, Dengue virus
  source 1..99: mol_type = other DNA
atgaaccaacgaaaaaaggtggttagaccacctttcaatatgctgaaacgcgagagaaaccgcgtatcaacccctcaagggttggtgaagagattctca

SEQ ID NO: 17 - DNA, 105 bp, Dengue virus
  source 1..105: mol_type = other DNA
attctgctgacatggctaggattaaactcaaggagcacgtccctttcaatgacgtgtatcgcagttggcatggtcacactgtacctaggagtcatggttcaggcg

SEQ ID NO: 18 - DNA, 105 bp, Dengue virus
  source 1..105: mol_type = other DNA
gtcattatcacatggataggaatgaattcacgcagcacctcactgtctgtgtcactagtattggtgggaattgtgacactgtatttgggagtcatggtgcaggcg

SEQ ID NO: 19 - DNA, 105 bp, Dengue virus
  source 1..105: mol_type = other DNA
gttctcttgacttggatagggttgaattcaaaaaacacatccatgtcattttcatgcattgcgataggagtcattacactctatctgggagctgtggtgcaagct

SEQ ID NO: 20 - DNA, 105 bp, Dengue virus
  source 1..105: mol_type = other DNA
ttcttagtgttgtggattggcacgaactcgaggaacacttcaatggctatgacgtgcatagctgttggaggaatcactctgtttctgggcttcacagttcaagca

SEQ ID NO: 21 - DNA, 80 bp, hepatitis delta virus
  source 1..80: mol_type = other DNA
gggtcggcatggcatctccacctcctcgcggtccgacctgggcatccgaaggaggacgcacgtccactcggatggctaag

SEQ ID NO: 22 - protein, 2617 aa, Dengue virus
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAGSGEVDSFSLGLLCISIMIEEVMRSRWSRKMLMTGTLAVFLLLTMGQLTWNDLIRLCIMVGANASDKMGMGTTYLALMATFRMRPMFAVGLLFRRLTSREVLLLTVGLSLVASVELPNSLEELGDGLAMGIMMLKLLTDFQSHQLWATLLSLTFVKTTFSLHYAWKTMAMILSIVSLFPLCLSTTSQKTTWLPVLLGSLGCKPLTMFLITENKIWGRKSWPLNEGIMAVGIVSILLSSLLKNDVPLAGPLIAGGMLIACYVISGSSADLSLEKAAEVSWEEEAEHSGASHNILVEVQDDGTMKIKDEERDDTLTILLKATLLAISGVYPMSIPATLFVWYFWQKKKQRSGVLWDTPSPPEVERAVLDDGIYRILQRGLLGRSQVGVGVFQEGVFHTMWHVTRGAVLMYQGKRLEPSWASVKKDLISYGGGWRFQGSWNAGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIKRKLRTLVLAPTRVVASEMAEALKGMPIRYQTTAVKSEHTGKEIVDLMCHATFTMRLLSPVRVPNYNMIIMDEAHFTDPASIAARGYISTRVGMGEAAAIFMTATPPGSVEAFPQSNAVIQDEERDIPERSWNSGYDWITDFPGKTVWFVPSIKSGNDIANCLRKNGKRVVQLSRKTFDTEYQKTKNNDWDYVVTTDISEMGANFRADRVIDPRRCLKPVILKDGPERVILAGPMPVTVASAAQRRGRIGRNQNKEGDQYIYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEIWTKEGERKKLRPRWLDARTYSDPLALREFKEFAAGRRSVSGDLILEIGKLPQHLTQRAQNALDNLVMLHNSEQGGKAYRHAMEELPDTIETLMLLALIAVLTGGVTLFFLSGRGLGKTSIGLLCVIASSALLWMASVEPHWIAASIILEFFLMVLLIPEPDRQRTPQDNQLAYVVIGLLFMILTVAANEMGLLETTKKDLGIGHAAAENHHHAAMLDVDLHPASAWTLYAVATTIITPMMRHTIENTTANISLTAIANQAAILMGLDKGWPISKMDIGVPLLALGCYSQVNPLTLTAAVLMLVAHYAIIGPGLQAKATREAQKRTAAGIMKNPTVDGIVAIDLDPVVYDAKFEKQLGQIMLLILCTSQILLMRTTWALCESITLATGPLTTLWEGSPGKFWNTTIAVSMANIFRGSYLAGAGLAFSLMKSLGGGRRGTGAQGETLGEKWKRQLNQLSKSEFNTYKRSGIIEVDRSEAKEGLKRGETTKHAVSRGTAKLRWFVERNLVKPEGKVIDLGCGRGGWSYYCAGLKKVTEVKGYTKGGPGHEEPIPMATYGWNLVKLYSGKDVFFTPPEKCDTLLCDIGESSPNPTIEEGRTLRVLKMVEPWLRGNQFCIKILNPYMPSVVETLEQMQRKHGGMLVRNPLSRNSTHEMYWVSCGTGNIVSAVNMTSRMLLNRFTMAHRKPTYERDVDLGAGTRHVAVEPEVANLDIIGQRIENIKNEHKSTWHYDEDNPYKTWAYHGSYEVKPSGSASSMVNGVVRLLTKPWDVIPMVTQIAMTDTTPFGQQRVFKEKVDTRTPKAKRGTAQIMEVTARWLWGFLSRNKKPRICTREEFTRKVRSNAAIGAVFVDENQWNSAKEAVEDERFWDLVHRERELHKQGKCATCVYNMMGKREKKLGEFGKAKGSRAIWYMWLGARFLEFEALGFMNEDHWFSRENSLSGVEGEGLHKLGYILRDISKIPGGNMYADDTAGWDTRITEDDLQNEAKITDIMEPEHALLATSIFKLTYQNKVVRVQRPAKNGTVMDVISRRDQRGSGQVGTYGLNTFTNMEAQLIRQMESEGIFSPSELETPNLAERVLDWLKKHGTERLKRMAISGDDCVVKPIDDRFATALTALNDMGKVRKDIPQWEPSKGWNDWQQVPFCSHHFHQLIMKDGREIVVPCRNQDELVGRARVSQGAGWSLRETACLGKSYAQMWQLMYFHRRDLRLAANAICSAVPVDWVPTSRTTWSIHAHHQWMTTEDMLSVWNRVWIEENPWMEDKTHVSSWEDVPYLGKREDQWCGSLIGLTARATWATNIQVAINQVRRLIGNENYLDFMTSMKRFKNESDPEGALW

SEQ ID NO: 23 - protein, 2616 aa, Dengue virus
DSGCVVSWKNKELKCGSGIFITDNVHTWTEQYKFQPESPSKLASAIQKAQEEGICGIRSVTRLENLMWKQITPELNHILAENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDAFCDSKLMSAAIKDNRAVHADMGYWIESALNDTWKIEKASFIEVKNCHWPKSHTLWSNGVLESEMIIPKNLAGPVSQHNYRPGYHTQIAGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVVVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLLLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFRTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYKVAAEGINYADRRWCFDGIKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALLLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMETLQRKYGGALVRNPLSRNSTHEMYWVSNASGNIVSSVNMISRMLINRFTMRHKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKNIQHLTVTEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLSVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 24 - protein, 2614 aa, Dengue virus
DMGCVINWKGKELKCGSGIFVTNEVHTWTEQYKFQADSPKRLATAIAGAWENGVCGIRSTTRMENLLWKQIANELNYILWENNIKLTVVVGDIIGVLEQGKRTLTPQPMELKYSWKTWGKAKIVTAEIQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWSNGVLESDMIIPKSLAGPISQHNYRPGYHTQTAGPWHLGKLDLDFNYCEGTTVVITENCGTRGPSLRTTTVSGKLIHEWCCRSCTLPPLRYMGEDGCWYGMEIRPISEKEENMVKSLVSAGSGKVDNFTMGVLCLAILFEEVMRGKFGKKHMIAGVFFTFVLLLSGQITWRDMAHTLIMIGSNASDKMGMGVTYLALIATFKIQPFLALGFFLRKLTSRENLLLGVGLAMATTLQLPEDIEQMANGIALGLMALKLITQFETYQLWTALISLTCSNTMFTLTVAWRTATLILAGVSLLPVCQSSSMRKTDWLPMAVAAMGVPPLPLFIFSLKDTLKRRSWPLNEGVMAVGLVSILASSLLRNDVPMAGPLVAGGLLIACYVITGTSADLTVEKAADITWEEEAEQTGVSHNLMITVDDDGTMRIKDDETENILTVLLKTALLIVSGVFPYSIPATLLVWHTWQKQTQRSGVLWDVPSPPETQKAELEEGVYRIKQQGIFGKTQVGVGVQKEGVFHTMWHVTRGAVLTYNGKRLEPNWASVKKDLISYGGGWRLSAQWQKGEEVQVIAVEPGKNPKNFQTMPGTFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRQTTATKSEHTGREIVDLMCHATFTMRLLSPVRVPNYNLIIMDEAHFTDPASIAARGYISTRVGMGEAAIFMTATPPGTADAFPQSNAPIQDEERDIPERSWNSGNEWITDFAGKTVWFPSIKAGNDIANCLRKNGKKVIQLSRKTFDTEYQKTKLNDWDFVVTTDISEMGANFKADRVIDPRRCLKPVILTDGPERVILAGPMPVTAASAAQRRGRVGRNPQKENDQYIFTGQPLNNDEDHAHWTEAKMLLDNINTPEGIIPALFEPEREKSAAIDGEYRLKGESRKTFVELMRRGDLPVWLAHKVASEGIKYTDRKWCFDGQRNNQILEENMDVEIWTKEGEKKKLRPRWLDARTYSDPLALKEFKDFAAGRKSIALDLVTEIGRVPSHLAHRTRNALDNLVMLHTSEHGGRAYRHAVEELPETMETLLLLGLMILLTGGAMLFLISGKGIGKTSIGLICVIASSGMLWMAEIPLQWIASAIVLEFFMMVLLIPEPEKQRTPQDNQLAYVVIGILTLAAIIAANEMGLLETTKRDLGMSKEPGVVSPTSYLDVDLHPASAWTLYAVATTVITPMLRHTIENSTANVSLAAIANQAVVLMGLDKGWPISKMDLGVPLLALGCYSQVNPLTLTAAVLLLITHYAIIGPGLQAKATREAQKRTAAGIMKNPTVDGIMTIDLDPVIYDSKFEKQLGQVMLLVLCAVQLLLMRTSWALCEALTLATGPITTLWEGSPGKFWNTTIAVSMANIFRGSYLAGAGLAFSIMKSVGTGKRGTGSQGETLGEKWKKKLNQLSRKEFDLYKKSGITEVDRTEAKEGLKRGETTHHAVSRGTAKLQWFVERNMVIPEGRVIDLGCGRGGWSYYCAGLKKVTEVRGYTKGGPGHEEPVPMSTYGWNIVKLMSGKDVFYLPPEKCDTLLCDIGESSPSPTVEESRTIRVLKMVEPWLKNNQFCIKVLNPYMPTVIEHLERLQRKHGGMLVRNPLSRNSTHEMYWISNGTGNIVASVNMVSRLLLNRFTMTHRRPTIEKDVDLGAGTRHVNAEPETPNMDVIGERIKRIKEEHNSTWHYDDENPYKTWAYHGSYEVKATGSASSMINGVVKLLTKPWDVVPMVTQMAMTDTTPFGQQRVFKEKVDTRTPRSMPGTRRVMGITAEWLWRTLGRNKKPRLCTREEFTKKVRTNAAMGAVFTEENQWDSAKAAVEDEDFWKLVDRERELHKLGKCGSCVYNMMGKREKKLGEFGKAKGSRAIWYMWLGARYLEFEALGFLNEDHWFSRENSYSGVEGEGLHKLGYILRDISKIPGGAMYADDTAGWDTRITEDDLHNEEKITQQMDPEHRQLANAIFKLTYQNKVVKVQRPTPTGTVMDIISRKDQRGSGQVGTYGLNTFTNMEAQLIRQMEGEGVLSKADLENPHLPEKKITQWLETKGVERLRRMAISGDDCVVKPIDDRFANALLALNDMGKVRKDIPQWQPSKGWHDWQQVPFCSHHFHELIMKDGRKLVVPCRPQDELIGRARISQGAGWSLRETACLGKAYAQMWSLMYFHRRDLRLASNAICSAVPVHWVPTSRTTWSIHAHHQWMTTEDMLTVWNRVWIEDNPWMEDKTPVTTWENVPYLGKREDQWCGSLIGLTSRATWAQNIPTAIQQVRSLIGNEEFLDYMPSMKRFRKEEESEGAIW

SEQ ID NO: 25 - protein, 2612 aa, Dengue virus
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAGQGTSETFSMGLLCLTLFVEECLRRRVTRKHMILVVVITLCAIILGGLTWMDLLRALIMLGDTMSGRIGGQIHLAIMAVFKMSPGYVLGVFLRKLTSRETALMVIGMAMTTVLSIPHDLMELIDGISLGLILLKIVTQFDNTQVGTLALSLTFIRSTMPLVMAWRTIMAVLFVVTLIPLCRTSCLQKQSHWVEITALILGAQALPVYLMTLMKGASRRSWPLNEGIMAVGLVSLLGSALLKNDVPLAGPMVAGGLLLAAYVMSGSSADLSLEKAANVQWDEMADITGSSPIVEVKQDEDGSFSIRDVEETNMITLLVKLALITVSGLYPLAIPVTMTLWYMWQVKTQRSGALWDVPSPAATKKAALSEGVYRIMQRGLFGKTQVGVGIHMEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAIFMTATPPGATDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILPDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYEDREWCFTGERNNQILEENMEVEIWTREGEKKKLRPRWLDARVYADPMALKDFKEFASGRKSITLDILTEIASLPTYLSSRAKLALDNIVMLHTTERGGRAYQHALNELPESLETLMLVALLGAMTAGIFLFFMQGKGIGKLSMGLITIAVASGLLWVAEIQPQWIAASIILEFFLMVLLIPEPEKQRTPQDNQLIYVILTILTIIGLIAANEMGLIEKTKTDFGFYQVKTETTILDVDLRPASAWTLYAVATTILTPMLRHTIENTSANLSLAAIANQAAVLMGLGKGWPLHRMDLGVPLLAMGCYSQVNPTTLTASLVMLLVHYAIIGPGLQAKATREAQKRTAAGIMKNPTVDGITVIDLEPISYDPKFEKQLGQVMLLVLCAGQLLLMRTTWAFCEVLTLATGPILTLWEGNPGRFWNTTIAVSTANIFRGSYLAGAGLAFSLIKNAQTPRRGTGTTGETLGEKWKRQLNSLDRKEFEEYKRSGILEVDRTEAKSALKDGSKIKHAVSRGSSKIRWIVERGMVKPKGKVVDLGCGRGGWSYYMATLKNVTEVKGYTKGGPGHEEPIPMATYGWNLVKLHSGVDVFYKPTEQVDTLLCDIGESSSNPTIEEGRTLRVLKMVEPWLSSKPEFCIKVLNPYMPTVIEELEKLQRKHGGNLVRCPLSRNSTHEMYWVSGASGNIVSSVNTTSKMLLNRFTTRHRKPTYEKDVDLGAGTRSVSTETEKPDMTIIGRRLQRLQEEHKETWHYDQENPYRTWAYHGSYEAPSTGSASSMVNGVVKLLTKPWDVIPMVTQLAMTDTTPFGQQRVFKEKVDTRTPQPKPGTRMVMTTTANWLWALLGKKKNPRLCTREEFISKVRSNAAIGAVFQEEQGWTSASEAVNDSRFWELVDKERALHQEGKCESCVYNMMGKREKKLGEFGRAKGSRAIWYMWLGARFLEFEALGFLNEDHWFGRENSWSGVEGEGLHRLGYILEEIDKKDGDLMYADDTAGWDTRITEDDLQNEELITEQMAPHHKILAKAIFKLTYQNKVVKVLRPTPRGAVMDIISRKDQRGSGQVGTYGLNTFTNMEVQLIRQMEAEGVITQDDMQNPKGLKERVEKWLKECGVDRLKRMAISGDDCVVKPLDERFGTSLLFLNDMGKVRKDIPQWEPSKGWKNWQEVPFCSHHFHKIFMKDGRSLVVPCRNQDELIGRARISQGAGWSLRETACLGKAYAQMWSLMYFHRRDLRLASMAICSAVPTEWFPTSRTTWSIHAHHQWMTTEDMLKVWNRVWIEDNPNMTDKTPVHSWEDIPYLGKREDLWCGSLIGLSSRATWAKNIHTAITQVRNLIGKEEYVDYMPVMKRYSAPSESEGVL

SEQ ID NO: 26 - protein, 35 aa, Dengue virus
MNNQRKKTGRPSFNMLKRARNRVSTVSQLAKARFS

SEQ ID NO: 27 - protein, 34 aa, Dengue virus
MNNQRKKARNTPFNMLKRERNRVSTVQQLTKRFS

SEQ ID NO: 28 - protein, 34 aa, Dengue virus
MNNQRKKAGKPSINMLKRVRNRVSTGSQLAKRFS

SEQ ID NO: 29 - protein, 33 aa, Dengue virus
MNQRKKVVRPPFNMLKRERNRVSTPQGLVKRFS

SEQ ID NO: 30 - protein, 35 aa, Dengue virus
ILLTWLGLNSRSTSLSMTCIAVGMVTLYLGVMVQA

SEQ ID NO: 31 - protein, 35 aa, Zika virus
VIITWIGMNSRSTSLSVSLVLVGIVTLYLGVMVQA

SEQ ID NO: 32 - protein, 35 aa, Dengue virus
VLLTWIGLNSKNTSMSFSCIAIGVITLYLGAVVQA

SEQ ID NO: 33 - protein, 35 aa, Dengue virus
FLVLWIGTNSRNTSMAMTCIAVGGITLFLGFTVQA

SEQ ID NO: 34 - DNA, 2381 bp, Zika virus
  source 1..2381: mol_type = other DNA
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaacaataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccaccaggaagctgcaaacgcggtcgcaaacctggttggaatcaagagaatacacaaagcacttgattagagtcgaaaattggatattcaggaaccctggcttcgcgttagcagcggccgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttctgacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccgataatgctgtcagttcatggctcccagcacagtgggatgatcgttaatgacacaggacatgaaactgatgagaatagagcgaaagttgagataacgcccaattcaccgagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacctgaaatgtcgcctgaaaatggataaacttagattgaagggcgtgtcatactccttgtgtactgcagcgttcacattcaccaagatcccggctgaaacactgcacgggacagtcacagtggagttacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgct

SEQ ID NO: 35 - protein, 795 aa, Zika virus
MKNPKKKSGGFRIVNMLKRGVARVSPFGGLKRLPAGLLLGHGPIRMVLAILAFLRFTAIKPSLGLINRWGSVGKKEAMETIKKFKKDLAAMLRIINARKEKKRRGADTSVGIVGLLLTTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVELQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 36 - protein, 119 aa, Zika virus
MKNPKKKSGGFRIVNMLKRGVARVSPFGGLKRLPAGLLLGHGPIRMVLAILAFLRFTAIKPSLGLINRWGSVGKKEAMETIKKFKKDLAAMLRIINARKEKKRRGADTSVGIVGLLLTT

SEQ ID NO: 37 - protein, 96 aa, Zika virus
AMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRR

SEQ ID NO: 38 - protein, 579 aa, Zika virus
AVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVELQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

(19) United States
(12) Patent Application Publication
(10) Pub. No.: US 2026/0306610 A1
(43) Pub. Date: Oct. 8, 2026

(54) Systems and Methods for Delivery of Viral Antigens

(71) Applicant: Veergen Inc., Fresno, CA (US)

(72) Inventors: Gustavo Garcia, Jr., Fresno, CA (US); Sanjay Chauhan, Fresno, CA (US)

(73) Assignee: Veergen Inc., Fresno, CA (US)

(21) Appl. No.: 19/631,969

(22) Filed: Mar. 27, 2026

Related U.S. Application Data
(60) Provisional application No. 63/780,107, filed on Mar. 28, 2025.

(51) Int. Cl. A61K 39/12; A61K 39/00; A61P 31/14; C12N 7/00; C12N 15/86

(52) U.S. Cl. CPC A61K 39/12; A61P 31/14; C12N 7/00; C12N 15/86; A61K 2039/53; A61K 2039/70; C12N 2770/24134; C12N 2770/24143; C12N 2770/24152

(57) ABSTRACT

Systems and methods for generating viral vectors, including systems and methods for generating Dengue vaccine are described. Generally, a viral vector can comprise structural proteins from a non-dengue Orthoflavivirus species and a genetic code for nonstructural proteins from dengue virus or a different Orthoflavivirus species. Viral vectors can be produced to yield vaccines to stimulate immune response to the Orthoflavivirus species providing genetic code for nonstructural proteins.

### CROSS REFERENCE TO RELATED APPLICATIONS

The current application claims the benefit of U.S. Provisional Application No. 63/780,107, entitled “Systems and Methods for Delivery of Viral Antigens”, filed Mar. 28, 2025, the disclosure of which is hereby incorporated by reference in its entirety.

### SEQUENCE LISTING

The instant application contains a Sequence Listing which has been submitted electronically in XML format and is hereby incorporated by reference in its entirety. The XML sequencing was created on Mar. 27, 2026, is named V29-09734.xml, and is 82,269 bytes in size.

### TECHNICAL FIELD

The disclosure is generally directed toward systems and methods delivery of dengue virus antigens, including vectors for vaccination.

### BACKGROUND

Dengue is a mosquito-borne disease that arises due to infection by the dengue virus (DENV). The virus is prevalent in the South and Central Americas, Asia, Africa, and Australia and spreading into North America and Europe due to expansion of the mosquito vector habitat. There are four major antigenically distinct serotypes DENV-1, DENV-2, DENV-3 and DENV-4, each having several subtypes.

The Flaviviridae family of viruses includes the dengue virus and several other pathogenic viruses, including many that are arthropod-borne. These viruses are small-enveloped with positive-sense RNA genomes of approximately 9.0-13 kb. The family include the genera of Orthoflavivirus (yellow fever virus, dengue virus, Zika virus, Japanese encephalitis virus, Powassan virus, West Nile virus and tick-borne encephalitis virus), Pestivirus (bovine viral diarrhea virus, classical swine fever virus, and border disease virus from sheep), Hepacivirus (hepatitis C virus, canine hepacivirus, equine hepacivirus, and GB virus B), and Pegivirus (human pegivirus, porcine pegivirus, and goose pegivirus).

### SUMMARY

Several embodiments are directed towards systems and methods for delivery of dengue virus antigens. In many embodiments, a system for delivery of dengue virus antigens comprises a replication-defective viral vector derived of a Flaviviridae virus. In several embodiments, a system for delivery of dengue virus antigens comprises a viral vector for expressing one or more nonstructural dengue proteins. In many embodiments, a viral vector comprises an outer envelope and packaged genetic material encoding one or more nonstructural proteins of dengue virus. In several embodiments, an outer envelope of a viral vector is derived from a virus of a the Orthoflavivirus genera. In many embodiments, an outer envelope of a viral vector is derived from a virus of a the Orthoflavivirus species that is not a dengue virus. In several embodiments, a viral vector comprises packaged genetic material encoding one or more nonstructural proteins of dengue virus and does not functionally encode any structural proteins of dengue virus.

Many embodiments are directed to a viral vector system comprising packaged genetic material encoding one or more dengue virus nonstructural proteins selected from: NS1, NS2A, NS2B, NS3, NS4A, NS4B, and NS5. In several embodiments, a viral vector system comprises packaged genetic material encoding two or more dengue virus nonstructural proteins selected from: NS1, NS2A, NS2B, NS3, NS4A, NS4B, and NS5. In some embodiments, a viral vector system comprises packaged genetic material encoding all of the following dengue virus nonstructural proteins: NS1, NS2A, NS2B, NS3, NS4A, NS4B, and NS5.

Several embodiments are directed to a multivalent viral vector system comprising packaged genetic material encoding one or more dengue virus nonstructural proteins of two or more serotypes. In many embodiments, a multivalent viral vector system comprises packaged genetic material encoding one or more dengue virus nonstructural proteins of three or more serotypes. In some embodiments, a multivalent viral vector system comprises packaged genetic material encoding one or more dengue virus nonstructural proteins of four serotypes. In some embodiments, a multivalent viral vector system comprises packaged genetic material encoding one or more dengue virus nonstructural proteins of two or more subtypes of a serotype.

### BRIEF DESCRIPTION OF THE DRAWINGS

The description and claims will be more fully understood with reference to the following figures and data graphs, which are presented as examples and should not be construed as a complete recitation of the scope of the disclosure.

FIGS. 1A and 1B provide schematics of the structure (FIG. 1A) and polyprotein processing (FIG. 1B) of Flaviviridae family viruses.

FIGS. 2A and 2B provide schematics of expression constructs for a vector and antigen expression construct (FIG. 2A) and heterologous packaging structure core construct (FIG. 2B).

FIGS. 3A and 3B provide a schematic of an example of a DVAX vaccine vector design and manufacturing process. FIG. 3A: Genomic organization of wild-type DENV virus, replication-deficient DVAX vector, and packaging construct are presented.

FIG. 3B: Workflow schematic illustrating the steps of vaccine particle generation, purification, quality control, and safety and efficacy assessment.

FIGS. 4A and 4B provides images of DVAX packaging and production. FIG. 4A: DVAX producer cells expressing DENV antigens (fluorescent signal) after transfection. FIG. 4B: Testing and antigen expression of produced DVAX particles. Vero cells were inoculated with DENV vaccine particles and fixed 48 hours post-inoculation. The cells were analyzed for DENV antigen expression (fluorescent signal). The data shows the viral vaccine is efficient in transducing cells and expressing DENV nonstructural proteins.

FIG. 5 provides a schematic illustration of pre-clinical safety and efficacy evaluation of DVAX vaccine in small animal model.

FIGS. 6A and 6B provide data on DVAX safety study in adult mice. FIG. 6A: Survival curve over 21-day period of immunized groups with DVAX vaccine candidate or phosphate-buffer saline (PBS). FIG. 6B: Mice body weights over 21-day period of DVAX vaccine candidate and phosphate-buffer saline (PBS) groups. No statistically significant weight change between vaccinated and unvaccinated groups was noted, suggesting the vaccine had no deleterious effects on animal health.

FIGS. 7A-7C provide data on pre-clinical efficacy study that demonstrates that DVAX2 provides effective protection against DENV2 and DENV3 infections. FIG. 7A: Mice spleen weights of vaccinated (DVAX2) versus unvaccinated (Unvac) groups 3 days post-challenge with WT DENV2 and DENV3. FIGS. 7B & 7C: Spleen viral load of unvaccinated and DVAX2 immunized mice that were challenged with wild-type DENV serotypes 2 or 3 (3 dpi). The DVAX2 vaccinated mice had no detectable level of DENV replication. A two-tailed unpaired, nonparametric Mann-Whitney test was conducted; ***, 10 P<0.0001, *, P<0.05.

### DETAILED DESCRIPTION

The description generally provides systems and methods related to the provision of dengue antigen delivery. Several embodiments are directed towards replication-deficient viral vectors that can be utilized for vaccination against the dengue virus. In many embodiments, a viral vector has an enveloped outer surface with structural proteins derived from an Orthoflavivirus virus. In many embodiments, one or more of the structural proteins of the outer surface are not derived from dengue virus. In many embodiments, a viral vector comprises an expression construct encoding one or more nonstructural dengue virus proteins. In many embodiments, a viral vector comprises an expression construct that does not functionally encode one or more structural dengue virus proteins.

Several embodiments are directed toward compositions of viral-vector vaccines and methods for administrating viral vectors. In many embodiments, a vaccine comprises viral vectors for delivery of expression constructs that express one or more nonstructural dengue virus proteins. In many embodiments, a vaccine is multivalent and comprises viral vectors that express one or more nonstructural dengue virus proteins of two or more serotypes.

Several embodiments are also directed towards systems and methods for production of viral vectors and vaccines. In many embodiments, a set of one or more expression cassettes are utilized to express structural proteins to yield an encapsulated and enveloped outer surface and an RNA to be packaged therein. In many embodiments, the set of one or more expression cassettes are expressed within packaging cells to yield high titers of viral vectors. In many embodiments, viral vectors are enriched and prepared for use as a vaccine.

### Orthoflavivirus

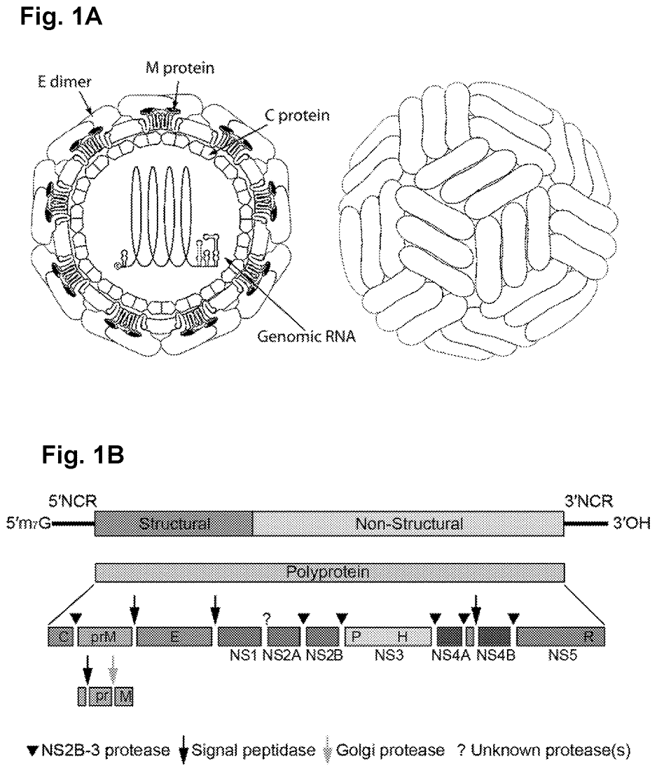

Provided in FIGS. 1A and 1B is a schematic of an Orthoflavivirus virion (FIG. 1A) and an expressed polyprotein of from the Orthoflavivirus genome (FIG. 1B). Three structural proteins make up the outer layer: capsid (C, 11 kDa), the major envelope protein (E, 50 kDa), and either prM (26 kDa), in immature virions, or M (8 kDa), in mature virions. These three structural proteins are near the N-terminus of the polypeptide. The Orthoflavivirus genome also expresses seven nonstructural proteins: NS1 (46 kDa), NS2A (22 kDa), NS2B (14 kDa), NS3 (70 kDa), NS4A (16 kDa), NS4B (27 kDa), and NS5 (103 kDa). The polypeptide is cleaved to yield the individualized peptides of the polypeptide. These viruses take advantage of host proteases as well as expressing its own protease, a function provided by NS2B-3. Signal peptidase, which is expressed within the endoplasmic reticulum, cleaves the peptide at several locations. The NS2B-3 protease self-cleaves to release itself and further cleaves at a number of other locations. Precursor M protein is cleaved by Golgi protease to yield mature M protein.

Several Orthoflavivirus species are well characterized, many of which are arthropod-borne. Examples include dengue virus (Orthoflavivirus dengue), Japanese encephalitis virus (Orthoflavivirus japonicum), Tick-borne encephalitis virus (Orthoflavivirus encephalitidis), Powassan virus (Orthoflavivirus powassanense), West Nile virus (Orthoflavivirus nilense), Yellow fever virus (Orthoflavivirus flavi), and Zika virus (Orthoflavivirus zikaense). A more complete list of Orthoflavivirus species can be found within T. S. Postler, et al., Arch Virol. 2023 Aug. 10; 168 (9): 224, the disclosure of which is hereby incorporated by reference.

Several Orthoflavivirus species comprise multiple antigenically distinct serotypes. For example, dengue virus comprises four distinct serotypes, referred to as DENV-1, DENV-2, DENV-3, and DENV-4.

### Systems of Viral Vectors

Several embodiments are directed to systems of viral vectors for delivery of dengue virus protein. Generally, a viral vector of the current disclosure comprises an inner space and an outer layer. In many embodiments, the inner space comprises a positive strand RNA for expressing antigenic proteins. In many embodiments, the outer core comprises structural proteins of Orthoflavivirus.

Various designs of viral vectors of the disclosure overcome common issues with dengue vaccines. A severe complication of dengue that can arise is secondary infection of a dengue virus. Specifically, when immune to a particular serotype of dengue, a secondary infection of a different serotype can result in heightened disease (much more severe than the first infection) due to a phenomenon known as antibody-dependent enhancement (ADE). Dengue viruses infect and replicates within various immune cells, including dendritic cells, macrophages, monocytes, and lymphocytes. Obtaining immunity results in development of antibodies that recognize the outer core structural proteins (e.g., neutralizing antibodies). Upon infection with a second serotype, these antibodies target the structural proteins of the dengue virions resulting in phagocytosis of these virions by dendritic cells and macrophages, increasing infection and replication of the dengue virus in its target cells. To mitigate the issue of ADE, various viral vectors are configured to exclude one or more dengue virus structural proteins, which can be replaced with structural proteins from another source. In many embodiments, non-dengue Orthoflavivirus structural proteins are utilized to encapsulate a viral vector for expressing dengue virus antigens. In certain embodiments, Zika virus structural proteins are utilized to encapsulate a viral vector for expressing dengue virus antigens.

In accordance with several embodiments, a viral vector for expressing dengue virus antigens comprises a positive strand RNA for expressing one or more nonstructural proteins of the dengue virus (or antigenic fragments thereof). The nonstructural proteins can stimulate an immune response when expressed within a mammalian host, which can be useful for vaccination. In several embodiments, the one or more nonstructural proteins of the dengue virus are selected from: NS1, NS2A, NS2B, NS3, NS4A, NS4B, and NS5.

In some embodiments, a viral vector comprises a positive strand RNA for expressing two or more nonstructural proteins of the dengue virus are selected from: NS1, NS2A, NS2B, NS3, NS4A, NS4B, and NS5. In some embodiments, a viral vector comprises a positive strand RNA for expressing three or more nonstructural proteins of the dengue virus are selected from: NS1, NS2A, NS2B, NS3, NS4A, NS4B, and NS5. In some embodiments, a viral vector comprises a positive strand RNA for expressing four or more nonstructural proteins of the dengue virus are selected from: NS1, NS2A, NS2B, NS3, NS4A, NS4B, and NS5. In some embodiments, a viral vector comprises a positive strand RNA for expressing five or more nonstructural proteins of the dengue virus are selected from: NS1, NS2A, NS2B, NS3, NS4A, NS4B, and NS5. In some embodiments, a viral vector comprises a positive strand RNA for expressing six or more nonstructural proteins of the dengue virus are selected from: NS1, NS2A, NS2B, NS3, NS4A, NS4B, and NS5. In some embodiments, a viral vector comprises a positive strand RNA for expressing all seven nonstructural proteins of the dengue virus: NS1, NS2A, NS2B, NS3, NS4A, NS4B, and NS5.

Many embodiments are directed towards viral vectors comprising an outer layer excluding one or more structural proteins of dengue virus, which can be utilized to express dengue virus antigens. In lieu of dengue virus structural proteins, viral vectors can be produced comprising an outer layer with one or more structural proteins of another Orthoflavivirus species, such as (for example) Zika virus. Accordingly, in some embodiments, a viral vector comprising a positive strand RNA for expressing one or more nonstructural proteins of the dengue virus comprises an outer layer that excludes one or more dengue virus structural proteins selected from: C, M, and E. In some embodiments, a viral vector comprising a positive strand RNA for expressing one or more nonstructural proteins of the dengue virus comprises an outer layer that excludes two or more dengue virus structural proteins selected from: C, M, and E. In some embodiments, a viral vector comprising a positive strand RNA for expressing one or more nonstructural proteins of the dengue virus comprises an outer layer that excludes the dengue virus structural proteins: C, M, and E.

In some embodiments, a viral vector comprising a positive strand RNA for expressing one or more nonstructural proteins of the dengue virus comprises an outer layer that includes one or more non-dengue-virus Orthoflavivirus structural proteins selected from: C, M, and E. In some embodiments, a viral vector comprising a positive strand RNA for expressing one or more nonstructural proteins of the dengue virus comprises an outer layer that includes two or more non-dengue-virus Orthoflavivirus structural proteins selected from: C, M, and E. In some embodiments, a viral vector comprising a positive strand RNA for expressing one or more nonstructural proteins of the dengue virus comprises an outer layer that includes non-dengue-virus Orthoflavivirus structural proteins: C, M, and E. Structural proteins of any other Orthoflavivirus species can be utilized, including (but not limited to) Japanese encephalitis virus (Orthoflavivirus japonicum), Tick-borne encephalitis virus (Orthoflavivirus encephalitidis), West Nile virus (Orthoflavivirus nilense), Powassan virus, Yellow fever virus (Orthoflavivirus flavi), and Zika virus (Orthoflavivirus zikaense) (for more Orthoflavivirus species options, see also, T. S. Postler, et al., Arch Virol. 2023 Aug. 10; 168(9):224, the disclosure of which is hereby incorporated by reference). The structural proteins utilized for a viral vector can be fully processed or any precursor of a processed protein. In some embodiments, the outer layer comprises non-dengue-virus Orthoflavivirus structural proteins: C, M, and E derived from the Orthoflavivirus same species. In some embodiments, the outer layer comprises non-dengue-virus Orthoflavivirus structural proteins: C, M, and E derived from two or more Orthoflavivirus species.

Many embodiments of viral vectors utilize Zika virus structural proteins for an outer capsid and envelope layer, which have been found to yield high titers of viral vectors for expressing viral antigens. In some embodiments, a viral vector for expressing viral antigens comprises an outer layer that comprises one or more Zika virus structural proteins: C, M, and E. In some embodiments, a viral vector for expressing viral antigens comprises an outer layer that comprises two or more Zika virus structural proteins: C, M, and E. In some embodiments, a viral vector for expressing viral antigens comprises an outer layer that comprises Zika virus structural proteins: C, M, and E. In many embodiments, a viral vector comprises Zika virus structural proteins for an outer capsid and envelope layer for expression of non-Zika virus nonstructural proteins of another Orthoflavivirus species (i.e., any Orthoflavivirus species other than Orthoflavivirus zikaense). In certain embodiments, a viral vector comprises Zika virus structural proteins for an outer capsid and envelope layer for expression of dengue virus nonstructural proteins.

Several embodiments of the disclosure are directed towards multivalent viral vector system. Generally, a multivalent viral vector system comprises viral vectors that express one or more nonstructural proteins of two or more Orthoflavivirus species, two or more serotypes of Orthoflavivirus species, or two or more subtypes of Orthoflavivirus species serotypes.

Several embodiments of the disclosure are directed towards multivalent viral vector system comprising viral vectors that express nonstructural proteins of multiple dengue virus serotypes. As previously noted, dengue viruses comprise four major serotypes DENV-1, DENV-2, DENV-3, and DENV-4. In some particular embodiments, a multivalent viral vector system comprises viral vectors that express one or more nonstructural proteins of two or more serotypes of dengue virus. In some particular embodiments, a multivalent viral vector system comprises viral vectors that express one or more nonstructural proteins of three or more serotypes of dengue virus. In some particular embodiments, a multivalent viral vector system comprises viral vectors that express one or more nonstructural proteins of all four serotypes of dengue virus.

Several embodiments of the disclosure are directed towards multivalent viral vector system comprising viral vectors that express nonstructural proteins of multiple genetic subtypes. Subtypes within a dengue virus serotype is a dengue virus that can be identified by a genotype. Five genetic subtypes have been described for DENV-1; five genetic subtypes have also been described for DENV-2; four genetic subtypes have been described for DENV-3; and four genetic subtypes have also been described for DENV-4 (see, e.g., S. C. Weaver and N. Vasilakis, Infect Genet Evol. 2009 July; 9(4):523-40; the disclosure of which is hereby incorporated by reference). In some particular embodiments, a multivalent viral vector system comprises viral vectors that express one or more nonstructural proteins of two or more subtypes of dengue virus serotypes. In some particular embodiments, a multivalent viral vector system comprises viral vectors that express one or more nonstructural proteins of three or more subtypes of dengue virus serotypes. In some particular embodiments, a multivalent viral vector system comprises viral vectors that express one or more nonstructural proteins of four or more subtypes of dengue virus serotypes. In some particular embodiments, a multivalent viral vector system comprises viral vectors that express one or more nonstructural proteins of five or more subtypes of dengue virus serotypes.

Multivalent viral vector systems can be implemented in a variety of ways. A multivalent viral vector system can comprise viral vectors sharing a common expression system shared by all the vectors; the expression system can provide a means for expressing two or more serotypes. This can be achieved by recombining two or more serotypes (or subtypes) into a singular expression system. In some implementations, a multivalent viral vector system is to utilize vectors that include a positive strand RNA for expressing nonstructural proteins of two or more serotypes of dengue virus. For example, a dengue virus multivalent viral vector system can comprise viral vectors in which each vector comprises a sequence encoding a common positive strand RNA for expressing a DENV-1 NS3 and a DENV-2 NS5 that is shared among the viral vectors.

Several embodiments of the disclosure are directed towards multivalent viral vector system comprising viral vectors in which each vector comprises a sequence encoding a common positive strand RNA for expressing nonstructural proteins of multiple dengue virus serotypes. In some embodiments, a multivalent viral vector system comprises viral vectors, each vector comprising a common positive strand RNA for expressing nonstructural proteins of two or more serotypes selected from: DENV-1, DENV-2, DENV-3, and DENV-4. In some embodiments, a multivalent viral vector system comprises viral vectors, each vector comprising a common positive strand RNA for expressing nonstructural proteins of three or more serotypes selected from: DENV-1, DENV-2, DENV-3, and DENV-4. In some embodiments, a multivalent viral vector system comprises viral vectors, each vector comprising a common positive strand RNA for expressing nonstructural proteins of four serotypes: DENV-1, DENV-2, DENV-3, and DENV-4.

Several embodiments of the disclosure are directed towards multivalent viral vector system comprising viral vectors in which each vector comprises a common positive strand RNA for expressing nonstructural proteins of multiple dengue virus subtypes. In some embodiments, a multivalent viral vector system comprises viral vectors, each vector comprising a common positive strand RNA for expressing nonstructural proteins of two or more subtypes of dengue virus serotypes. In some embodiments, a multivalent viral vector system comprises viral vectors, each vector comprising a common positive strand RNA for expressing nonstructural proteins of three or more subtypes of dengue virus serotypes. In some embodiments, a multivalent viral vector system comprises viral vectors, each vector comprising a common positive strand RNA for expressing nonstructural proteins of four or more subtypes of dengue virus serotypes. In some embodiments, a multivalent viral vector system comprises viral vectors, each vector comprising a common positive strand RNA for expressing nonstructural proteins of five or more subtypes of dengue virus serotypes.

Another way to implement a multivalent viral vector system is to utilize mixtures of viral vectors in which the mixture comprises two or more vector genotypes in which each vector genotype comprises a positive strand RNA for expressing one or more nonstructural proteins of a singular serotype (and/or subtype). A vector genotype is to refer to a vector having a unique genetic sequence. For example, a dengue virus multivalent viral vector system comprises a mixture of viral vector genotypes in which a percentage of the vectors comprise a vector genotype having a positive strand RNA for expressing one or more nonstructural proteins of DENV-1 and a percentage of the vectors comprise a vector genotype having a positive strand RNA for expressing one or more nonstructural proteins of DENV-2.

Several embodiments of the disclosure are directed towards multivalent viral vector system comprising multiple viral vector genotypes in which each vector genotype comprises a common positive strand RNA for expressing nonstructural proteins of a dengue virus serotype. In some embodiments, a multivalent viral vector system comprises viral vectors of two or more vector genotypes, each vector genotype comprising a positive strand RNA for expressing one or more nonstructural proteins of a genotype of a unique dengue virus serotype selected from: DENV-1, DENV-2, DENV-3, and DENV-4. In some embodiments, a multivalent viral vector system comprises viral vectors of three or more vector genotypes, each vector genotype comprising a positive strand RNA for expressing one or more nonstructural proteins of a genotype of a unique dengue virus serotype selected from: DENV-1, DENV-2, DENV-3, and DENV-4. In some embodiments, a multivalent viral vector system comprises viral vectors of four vector genotypes, each vector genotype comprising a positive strand RNA for expressing one or more nonstructural proteins of a genotype of a unique dengue virus serotype selected from: DENV-1, DENV-2, DENV-3, and DENV-4.

Several embodiments of the disclosure are directed towards multivalent viral vector system comprising multiple viral vector genotypes in which each vector genotype comprises a common positive strand RNA for expressing nonstructural proteins of a dengue virus subtype. In some embodiments, a multivalent viral vector system comprises viral vectors of two or more vector genotypes, each vector genotype comprising a positive strand RNA for expressing one or more nonstructural proteins of a genotype of a unique subtype of a dengue virus serotype. In some embodiments, a multivalent viral vector system comprises viral vectors of three or more vector genotypes, each vector genotype comprising a positive strand RNA for expressing one or more nonstructural proteins of a genotype of a unique subtype of a dengue virus serotype. In some embodiments, a multivalent viral vector system comprises viral vectors of four or more vector genotypes, each vector genotype comprising a positive strand RNA for expressing one or more nonstructural proteins of a genotype of a unique subtype of a dengue virus serotype. In some embodiments, a multivalent viral vector system comprises viral vectors of five or more vector genotypes, each vector genotype comprising a positive strand RNA for expressing one or more nonstructural proteins of a genotype of a unique subtype of a dengue virus serotype.

Multivalent viral vector systems can also be implemented by combining strategies. In some implementations, a multivalent viral vector system comprises two or more vector genotypes, each vector genotype comprising a positive strand RNA for expressing nonstructural proteins of two or more genotypes of a unique dengue virus serotype (or subtypes). Accordingly, various embodiments of multivalent viral vector systems for dengue virus can comprise two or more vector genotypes, each vector genotype comprising a positive strand RNA for expressing nonstructural proteins of two or more genotypes of a unique dengue virus serotype selected from: DENV-1, DENV-2, DENV-3, and DENV-4. Various embodiments of multivalent viral vector systems for dengue virus can comprise two or more vector genotypes, each vector genotype comprising a positive strand RNA for expressing nonstructural proteins of two or more genotypes of a unique subtype of a dengue virus serotype.

### Systems and Methods for Production of Viral Vectors and Vaccines

Several embodiments are directed towards systems and methods for production of viral vectors. Generally, nucleic acid expression constructs are expressed within production cells to yield high titers of replication-deficient viral vectors. The viral vectors can be collected and enriched, then prepared for further analysis and/or vaccine administration.

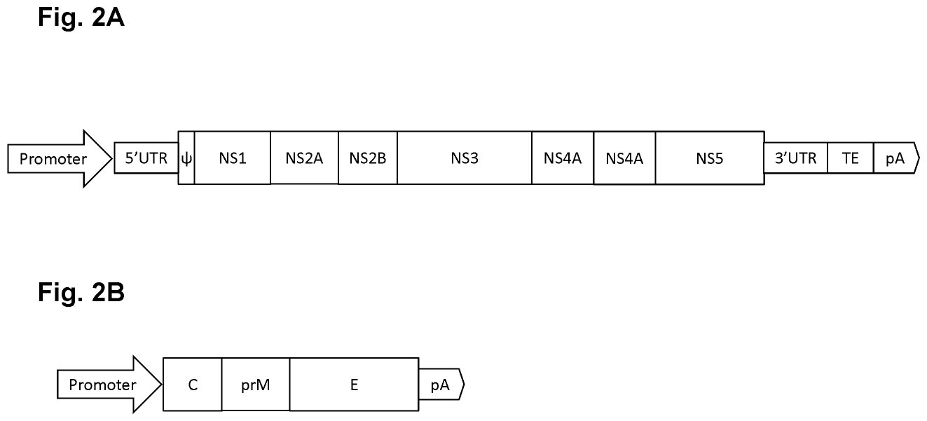

Provided in FIGS. 2A and 2B are examples of expression constructs that can be utilized to generate a viral vector. Each of the depicted constructs comprise a polygene sequence for encoding a polypeptide, operatively linked to regulatory sequences for RNA and/or peptide expression. FIG. 2A provides an example of an expression construct to yield a positive strand RNA to be packaged within an inner space of a viral vector. FIG. 2B provides an example of an expression construct to yield expression of structural peptides to form an outer layer of a viral vector.

In the example of the expression construct to yield a positive strand RNA to be packaged (FIG. 2A), the construct yields an RNA capable of being packaged into an outer capsid and envelope layer within packaging cells and capable of being further translated into the polypeptide upon transduction within a vaccine recipient. The construct includes an encoded nucleic acid sequence of one or more nonstructural proteins of dengue virus; the example within FIG. 2A includes all 7 nonstructural proteins. The construct further includes a 5′-UTR and 3′-UTR, which can be derived from the native sequence of an Orthoflavivirus species. The 5′-UTR or 3′-UTR can optionally include a translation enhancer (TE), which can help increase expression of the antigenic polypeptide. The construct can include a packaging signal sequence (ψ) such that it can be efficiently packaged into the viral vector. For Orthoflavivirus species, a packaging signal sequence (ψ) can be within the sequence of a native 5′-UTR. Examples of polypeptide sequences of nonstructural dengue virus proteins of a genotype for each serotype are provided within SEQ ID NOs: 22-25 (Table 2). Examples of a nucleotide sequence that encode nonstructural dengue virus proteins a genotype for each serotype are provided within SEQ ID NOs: 1-4 (Table 2). An example of a translational enhancer is provided within SEQ ID NO: 21 (Table 2).

In the example of the expression construct to yield structural peptides (FIG. 2B), the construct yields an RNA capable of being translated into polypeptide within the packaging cells. The construct includes a gene sequence of one or more structural proteins of a non-dengue-virus Orthoflavivirus species (e.g., Zika virus structural proteins). The construct should exclude a packaging signal sequence (w) to prevent the expressed RNA from being packaged into the viral vector. Examples of polypeptide sequences of structural zika virus proteins are provided within SEQ ID NOs: 35-38. Examples of a nucleotide sequence that encode structural zika virus proteins is provided within SEQ ID NO: 34.

In some embodiments, a gene or polygene is codon optimized for host expression (as may be dependent on intended vaccine recipient). In some embodiments, the transgene comprises a sequence for a peptide tag, a self-cleavage peptide, a signal peptide, or other peptide. Peptides that can be encoded with the transgene include (but are not limited to) His-tag, Myc-tag, HA-tag, FLAG-tag, VSV-tag, T7-tag, biotinylation site, Fc-tag, glutathione-S-transferase-tag, SNAP-tag, CLIP-tag, T2A self-cleavage peptide, F2A self-cleavage peptide, secretion signal peptide, nuclear localization signal, etc.

In some embodiments, any particular nucleotide or amino acid sequence may have a sequence that is modified from its native sequence. Alterations in sequence may be employed for any number of reasons, such as (for example) for codon optimization or to distinguish from native sequence of a biological cell, peptide mimics that have enhance activity, and sequences that are immunoevasive or immunogenic. In various embodiments, the amino acid or nucleobase sequence of a particular sequence is at least 50% identical to its native sequence, at least 60% identical to its native sequence, at least 70% identical to its native sequence, at least 80% identical to its native sequence, at least 90% identical to its native sequence, at least 95% identical to its native sequence, at least 99% identical to its native sequence, or 100% identical to its native sequence.

In some embodiments, an expression construct comprises a nucleotide sequence for encoding nonstructural proteins of DENV-1. In various embodiments, an expression construct comprises a nucleotide sequence for encoding nonstructural proteins of DENV-1 in which the amino acid sequence that is encoded is at least 50% identical, at least 60% identical, at least 70% identical, at least 80% identical, at least 90% identical, at least 95% identical, at least 99% identical, or 100% identical to SEQ ID NO: 22. In various embodiments, an expression construct comprises a nucleotide sequence for encoding nonstructural proteins of DENV-1, the nucleotide sequence is at least 50% identical, at least 60% identical, at least 70% identical, at least 80% identical, at least 90% identical, at least 95% identical, at least 99% identical, or 100% identical to SEQ ID NO: 1.

In some embodiments, an expression construct comprises a nucleotide sequence for encoding nonstructural proteins of DENV-2. In various embodiments, an expression construct comprises a nucleotide sequence for encoding nonstructural proteins of DENV-2 in which the amino acid sequence that is encoded is at least 50% identical, at least 60% identical, at least 70% identical, at least 80% identical, at least 90% identical, at least 95% identical, at least 99% identical, or 100% identical to SEQ ID NO: 23. In various embodiments, an expression construct comprises a nucleotide sequence for encoding nonstructural proteins of DENV-2, the nucleotide sequence is at least 50% identical, at least 60% identical, at least 70% identical, at least 80% identical, at least 90% identical, at least 95% identical, at least 99% identical, or 100% identical to SEQ ID NO: 2.

In some embodiments, an expression construct comprises a nucleotide sequence for encoding nonstructural proteins of DENV-3. In various embodiments, an expression construct comprises a nucleotide sequence for encoding nonstructural proteins of DENV-3 in which the amino acid sequence that is encoded is at least 50% identical, at least 60% identical, at least 70% identical, at least 80% identical, at least 90% identical, at least 95% identical, at least 99% identical, or 100% identical to SEQ ID NO: 24. In various embodiments, an expression construct comprises a nucleotide sequence for encoding nonstructural proteins of DENV-3, the nucleotide sequence is at least 50% identical, at least 60% identical, at least 70% identical, at least 80% identical, at least 90% identical, at least 95% identical, at least 99% identical, or 100% identical to SEQ ID NO: 3.

In some embodiments, an expression construct comprises a nucleotide sequence for encoding nonstructural proteins of DENV-4. In various embodiments, an expression construct comprises a nucleotide sequence for encoding nonstructural proteins of DENV-4 in which the amino acid sequence that is encoded is at least 50% identical, at least 60% identical, at least 70% identical, at least 80% identical, at least 90% identical, at least 95% identical, at least 99% identical, or 100% identical to SEQ ID NO: 25. In various embodiments, an expression construct comprises a nucleotide sequence for encoding nonstructural proteins of DENV-4, the nucleotide sequence is at least 50% identical, at least 60% identical, at least 70% identical, at least 80% identical, at least 90% identical, at least 95% identical, at least 99% identical, or 100% identical to SEQ ID NO: 4.

As noted herein, various embodiments of viral vectors exclude dengue virus structural proteins in the outer capsid and envelope layers. Accordingly, in some embodiments, an expression construct to yield packageable positive-strand RNA excludes nucleotide sequence for encoding a functional structural protein dengue virus. In some embodiments, an expression construct to yield packageable positive-strand RNA excludes nucleotide sequence for encoding functional dengue virus structural proteins: C, M, and E.

Although an expression construct to yield packageable positive-strand RNA excludes nucleotide sequence for encoding a functional structural protein dengue virus, it may include nucleotide sequences to yield a partial structural protein of dengue virus that is nonfunctional. It may be beneficial to include sequences of partial structural protein of dengue virus to promote high vector titer yields and/or high expression of non-structural proteins upon transduction. For example, it has been found that an expression construct that encodes a partial C protein and/or a partial E protein can yield high viral vector titers and robust nonstructural protein expression upon transduction. Examples of polypeptide sequences for partial and nonfunctional dengue virus C proteins of a genotype for each serotype are provided within SEQ ID NOs: 26-29 (Table 2). Examples of polypeptide sequences for partial and nonfunctional dengue virus E proteins of a genotype for each serotype are provided within SEQ ID NOs: 30-33 (Table 2). Examples of a nucleotide sequence that encode partial and nonfunctional dengue virus C proteins of a genotype for each serotype are provided within SEQ ID NOs: 13-16 (Table 2). Examples of a nucleotide sequence that encode partial and nonfunctional dengue virus E proteins of a genotype for each serotype are provided within SEQ ID NOs: 17-20 (Table 2).

In various embodiments, an expression construct comprises a 5′-UTR derived from DENV-1, DENV-2, DENV-3, or DENV-4. In various embodiments, an expression construct comprises a 5′-UTR derived from DENV-1, the sequence is at least 50% identical, at least 60% identical, at least 70% identical, at least 80% identical, at least 90% identical, at least 95% identical, at least 99% identical, or 100% identical to SEQ ID NO: 5. In various embodiments, an expression construct comprises a 5′-UTR derived from DENV-2, the sequence is at least 50% identical, at least 60% identical, at least 70% identical, at least 80% identical, at least 90% identical, at least 95% identical, at least 99% identical, or 100% identical to SEQ ID NO: 6. In various embodiments, an expression construct comprises a 5′-UTR derived from DENV-3, the sequence is at least 50% identical, at least 60% identical, at least 70% identical, at least 80% identical, at least 90% identical, at least 95% identical, at least 99% identical, or 100% identical to SEQ ID NO: 7. In various embodiments, an expression construct comprises a 5′-UTR derived from DENV-4, the sequence is at least 50% identical, at least 60% identical, at least 70% identical, at least 80% identical, at least 90% identical, at least 95% identical, at least 99% identical, or 100% identical to SEQ ID NO: 8. A 5′-UTR or one serotype can be used with nonstructural proteins and/or with a 3′-UTR of another serotype.

In various embodiments, an expression construct comprises a 3′-UTR derived from DENV-1, DENV-2, DENV-3, or DENV-4. In various embodiments, an expression construct comprises a 3′-UTR derived from DENV-1, the sequence is at least 50% identical, at least 60% identical, at least 70% identical, at least 80% identical, at least 90% identical, at least 95% identical, at least 99% identical, or 100% identical to SEQ ID NO: 9. In various embodiments, an expression construct comprises a 3′-UTR derived from DENV-2, the sequence is at least 50% identical, at least 60% identical, at least 70% identical, at least 80% identical, at least 90% identical, at least 95% identical, at least 99% identical, or 100% identical to SEQ ID NO: 10. In various embodiments, an expression construct comprises a 3′-UTR derived from DENV-3, the sequence is at least 50% identical, at least 60% identical, at least 70% identical, at least 80% identical, at least 90% identical, at least 95% identical, at least 99% identical, or 100% identical to SEQ ID NO: 11. In various embodiments, an expression construct comprises a 3′-UTR derived from DENV-4, the sequence is at least 50% identical, at least 60% identical, at least 70% identical, at least 80% identical, at least 90% identical, at least 95% identical, at least 99% identical, or 100% identical to SEQ ID NO: 12. A 3′-UTR or one serotype can be used with a 5′-UTR and/or nonstructural proteins of another serotype.

Several embodiments are directed towards an expression construct to yield a positive-strand RNA for packaging into a viral vector (viral particle), the expression construction comprising a 5′-UTR, nucleotide sequence encoding a set of one or more nonstructural proteins, and a 3′UTR. In various embodiments, an expression construct to yield a positive-strand RNA for packaging into a viral vector (viral particle), the expression construction comprising a 5′-UTR, nucleotide sequence encoding nonstructural proteins, and a 3′UTR; the 5′-UTR having a sequence that is at least 50% identical, at least 60% identical, at least 70% identical, at least 80% identical, at least 90% identical, at least 95% identical, at least 99% identical, or 100% identical to one of SEQ ID NOs: 5-8; the nucleotide sequence encoding nonstructural proteins having a sequence that is at least 50% identical, at least 60% identical, at least 70% identical, at least 80% identical, at least 90% identical, at least 95% identical, at least 99% identical, or 100% identical to one of SEQ ID NOs: 22-25; and the 3′-UTR having a sequence that is at least 50% identical, at least 60% identical, at least 70% identical, at least 80% identical, at least 90% identical, at least 95% identical, at least 99% identical, or 100% identical to one of SEQ ID NOs: 9-13.

Each construct can be operatively linked to a promoter and polyA signal to yield high RNA expression. The construction can optionally further include one or more operatively linked regulatory sequences: one or more enhancer sequences, a transcriptional termination sequence, a complete intron sequence containing a donor and acceptor splice site, a Kozak sequence, an internal ribosome entry site, or any other regulatory sequence for modulating, increasing, and/or controlling gene expression. The term “operatively linked” refers to a juxtaposition of a regulatory sequence with a transgene permitting them to function in their intended manner. Examples of mammalian regulatory sequences that can be utilized include (but are not limited to): CMV-promoter, SV40-promoter, RSV-promoter, EF1a-promoter, PGK1-promoter, Ubc-promoter, CMV-enhancer, SV40-enhancer, a globin intron, SV40 poly-A signal, and tk-poly-A signal. Expression constructs can also optionally include a viral origin of replication, a selectable marker, or an expression marker (e.g., fluorescent protein).

Expression constructs can be provided within any nucleic acid conformation, such as RNA expression vectors, linear DNA expression vectors, and plasmid DNA. Plasmid DNA is often preferred to facilitate high yield of DNA expression vectors utilizing bacterial systems. Plasmids can include an origin of replication, a selectable marker, cloning sites, or any other sequence utilized in the provision of molecular biology.

Viral vectors can be packaged within mammalian cell lines, insect cell lines, or any other appropriate cell line. Examples of cell lines include (but are not limited to): such as HEK293 cells, HEK293FT cells, Vero cells, BHK-21 cells, Caco-2 cells, Hela cells, plasma cells, Chinese Hamster Ovary (CHO) cells, HuH7 cells, C6/36 mosquito cells, or any other cells utilized for viral vector production. The cells can be grown in adherent or nonadherent conditions, on tissue culture plates or within bioreactors, and at any appropriate scale for testing or manufacturing.

To package viral vectors, packaging cells are contacted with expression constructs such that they enter therein and express within the cell (e.g., via transfection). Suitable methods for nucleic acid delivery into a packaging cell can include any method by which a nucleic acid can be introduced into a cell, a tissue or an organism. Examples of methods for nucleic acid delivery include (but are not limited to) direct delivery of DNA such as by injection, including microinjection; by electroporation; by calcium phosphate precipitation; by using a chemical carrier such as diethylaminomethyl (DEAE), polyethylene glycol (PEG), or polyethyleneimine (PEI); by direct sonic loading; by liposome mediated transfection; by microprojectile bombardment; and/or by agitation with silicon carbide fibers.

For stable, long-term transfection of mammalian cells, depending upon the expression vector and transfection technique used, typically only a fraction of cells (i.e., not 100%) will integrate the foreign DNA into their genome. In order to identify stably expressing cells within a population, a selectable marker (e.g., gene product inducing resistance to antibiotics or a fluorescent protein) can be introduced into the host cells along with the gene of interest. Cells stably transfected with the introduced nucleic acid can be identified by drug selection (e.g., cells that have incorporated the selectable marker gene will survive, while the other cells die) or identifying and isolating fluorescent cells (e.g., via flow cytometry) or identifying and isolating cells with extracellular expression of a transgene product (e.g., via flow cytometry and tagging cells with fluorescent antibodies capable of detecting the transgene product), among other methods known in the arts.

Several embodiments are directed towards production of vaccines. Upon expression of nucleic acid within producer cell, viral the viral vectors (viral vaccine particles) can be harvested from the cells by collecting the culture supernatant and/or cell lysate. Cellular components can be removed and the viral vectors can then be enriched and/or purified. Any appropriate method for viral vector enrichment and/or purification can be utilized. Examples of techniques utilized for viral vector enrichment and/or purification include (but are not limited to): nuclease treatment to remove excess nucleic acids, ultrafiltration, diafiltrating, antibody column exchange, chromatography, ultracentrifugation, Cs—Cl density gradient ultracentrifugation, dialysis, and sterile filtration.

Viral vectors can be left in suspension or lyophilized for storage. Storage solutions may include saline, a buffer, preservatives, hexose, or other components to protect viral vector transduction efficiency. Viral vectors may be stored at an appropriate temperature, such as −70° C. to −85° C., −15° C. to −25° C., or 2° C. to 8° C. or 24° C. to 37° C.

Viral vectors can be titered at various stages of production. A variety of methods can be utilized for assessing viral vector titer, such as (for example) measuring plaque forming units, quantifying viral genomes via RT-qPCR, q-PCR, fluorescent protein expression, immunofluorescence, enzyme-linked immunosorbent assay (ELISA), and dot plots.

Several embodiments are directed toward administration of viral vectors as vaccines to recipients. Generally, one or more doses of viral vectors can be administered to a recipient to induce an immune response. Dosage can be determined utilizing animal studies (e.g., mice) and within clinical trials. Generally, a dose can be within 10\hat{ }3 PFU to 10\hat{ }13 PFU. In various embodiments, a dose of viral vector for vaccination is between 10\hat{ }2 PFU and 10\hat{ }4 PFU, between 10\hat{ }3 PFU and 10\hat{ }5 PFU, 10\hat{ }4 PFU and 10\hat{ }6 PFU, between 10\hat{ }5 PFU and 10\hat{ }7 PFU, 10\hat{ }6 PFU and 10\hat{ }8 PFU, between 10\hat{ }7 PFU and 10\hat{ }9 PFU, 10\hat{ }8 PFU and 10\hat{ }10 PFU, between 10\hat{ }9 PFU and 10\hat{ }11 PFU, 10\hat{ }10 PFU and 10\hat{ }12 PFU, between 10\hat{ }11 PFU and 10\hat{ }13 PFU, or between 10\hat{ }12 PFU and 10\hat{ }14 PFU.

Administration of a vaccine can be repeated as necessary to yield an initial immune response or to boost a prior immune response. A number of vaccine schedules can be utilized and can be determined utilizing animal studies and within clinical trials. In some implementations, a single administration is performed to initiate immunity. In some implementations, two administrations are performed to initiate immunity; each dose can be separated by 2 weeks up to one year. In some implementations, three administrations are performed to initiate immunity; each dose can be separated by 2 weeks up to one year. In some implementations, a booster vaccine is performed after initiation of vaccination; a booster vaccine can be performed beyond a year of from receiving the initial vaccine, beyond a two years of from receiving the initial vaccine, beyond a five years of from receiving the initial vaccine, or beyond a 10 years of from receiving the initial vaccine.

Administration of a vaccine can be by any appropriate route. Examples of routes of administration include (but are not limited to): intramuscular, subcutaneous, and intranasal. Dilution and/or reconstitution of a stored vaccine formula can optionally be performed at the site of administration.

### EXAMPLES & EXPERIMENTAL DATA

Biological data enable and support the systems and methods for viral vector production and vaccine administration. The example utilizes a viral vector comprising dengue virus nonstructural proteins and Zika structural proteins in inducing immunity against dengue virus. Vaccine safety and efficacy assessment was performed on mice.

Described in this example is a Dengue vaccine platform (DVAX) based on utilizing an RNA replicon coding for viral antigens that are packaged in a viral shell and aimed at inducing long-lasting immune response. In this vaccine technology, the Dengue viral structural proteins are excluded that have been shown to induce sub-optimal antibodies that can enhance the severity of subsequent natural infection with different serotypes. The phenomenon is referred to as antibody-dependent enhancement (ADE).

Antibody-dependent enhancement was addressed by generating a Dengue vector construct containing only the nonstructural (NS) coding region flanked by 5′-untranslated (UTR) and 3′ UTR sequences. Using a packaging construct, this replication-deficient Dengue vaccine particles were generated in human producer cells and verified the production of viral particles and Dengue antigen expression in vitro. Our safety and efficacy animal study showed this Dengue vaccine (VGN-001) effectively inhibited infection of multiple Dengue serotypes without causing ADE.

### Results

### Generation of DVAX2 Replication-Deficient Vaccine

Rationale: The replication-deficient Dengue virus (DENV) vaccine (DVAX) is designed to enter the host cells upon immunization and express nonstructural (NS) viral proteins but not DENV structural proteins. Endogenous expression of NS proteins is expected to potentially present the peptides from those viral proteins on major histocompatibility complex (MHC) class I and induce predominantly a cell-mediated immune response. The intent is to induce cellular immunity against DENV NS proteins and examine its immune protective effects in animal model DENV infection.

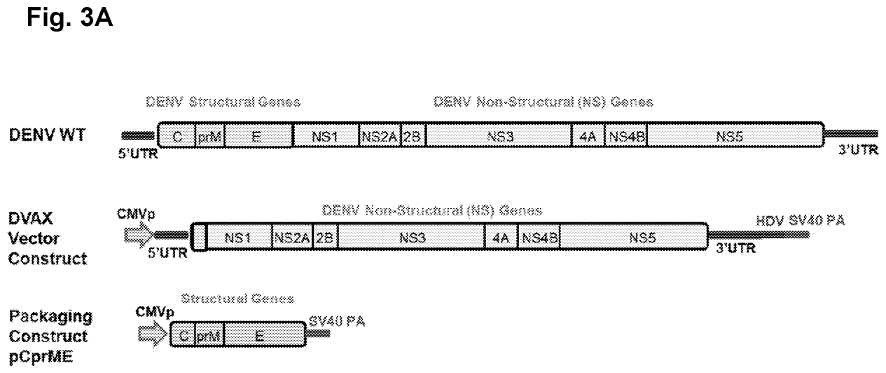

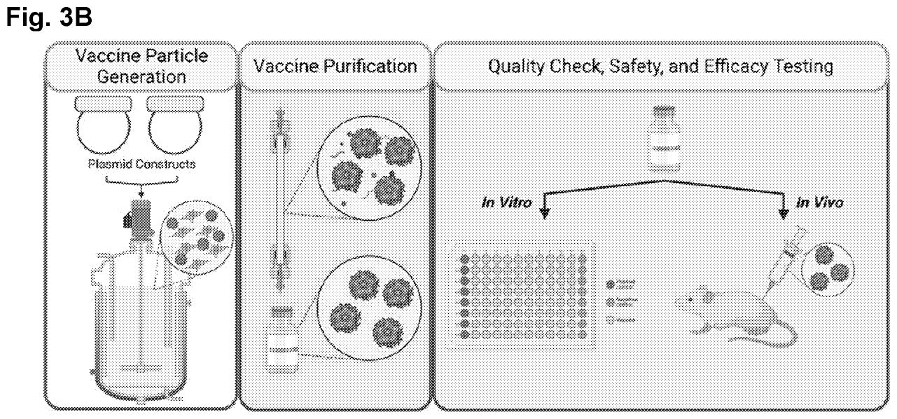

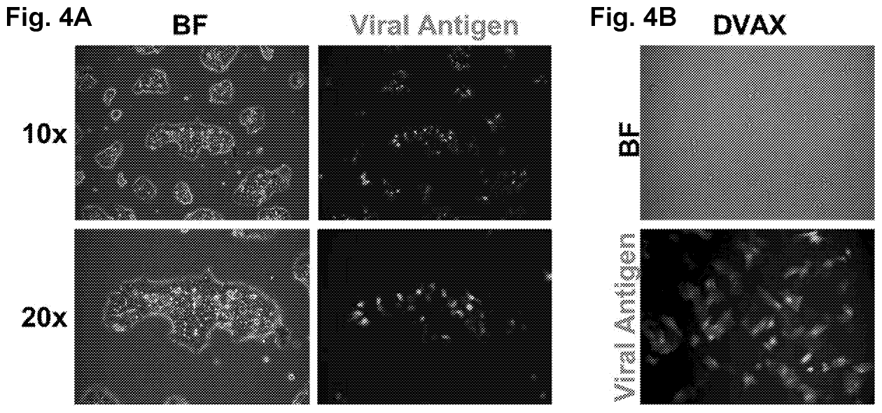

Design and Vector Construct: The replication-deficient DENV vector system was developed by removing the DENV structural coding gene region (For example, DENV2 genome location 199 to 2316 nucleotide were deleted based on the available DENV reference genome sequence: GenBank: GU289914.1) as described in FIG. 3A. This resulted in the removal of a segment comprised of 2,117 nucleotides, encompassing the core (C), pre-membrane (PrM), and envelope (E) structural genes. The DENV vector system genomic insert is flanked by a cytomegalovirus (CMV) promoter at the 5′ end, and a translational enhancer ribozyme sequence from hepatitis delta virus (HDV) followed by a SV40 poly(A) sequence at the 3′ end. A heterologous packaging plasmid DNA construct containing Zika viral core (C), precursor membrane (PrM), and envelope (E) structural genes on an expression plasmid. The schematic of vaccine particle generation, purification, quality testing, and safety and efficacy assessment is provided in FIG. 3B. To generate vaccine particles, the two-plasmid system comprising of the packaging plasmid (Zika structural genes [CprME]) and DVAX vector plasmid were transfected into producer cells at a ratio of 3:1. The transfection efficiency was observed by immunostaining the transfected cells and visualized under fluorescent microscopy (FIG. 4A). The purified vaccine particles were subjected to titer quantification and the transduced Vero cells were immunostained and visualized for DENV viral antigens using fluorescent microscopy (FIG. 4B). Cell culture supernatant was collected daily from Day 1 to Day 21 days post-transfection (dpt). The vaccine particles were subjected to purification. To confirm the titer and viral particle production efficiency, Vero cells were transduced with produced DVAX2 vaccine particles at a 10-fold dilution. Fluorescent microscopy visualized the expression of immunostained viral antigens in the cells at 48 hours post-transduction (FIG. 4B). The viral antigen expression (green) is mediated by NS proteins of DVAX. Therefore, our microscopy data indicates endogenous expression of the DENV NS proteins in transduced cells. In conclusion, DENV vaccines can be produced comprising viral vector particles that comprise a non-dengue virus outer surface with a positive-strand RNA genome for expressing non-structural proteins of DENV.

### DVAX is Safe in Mouse Model.

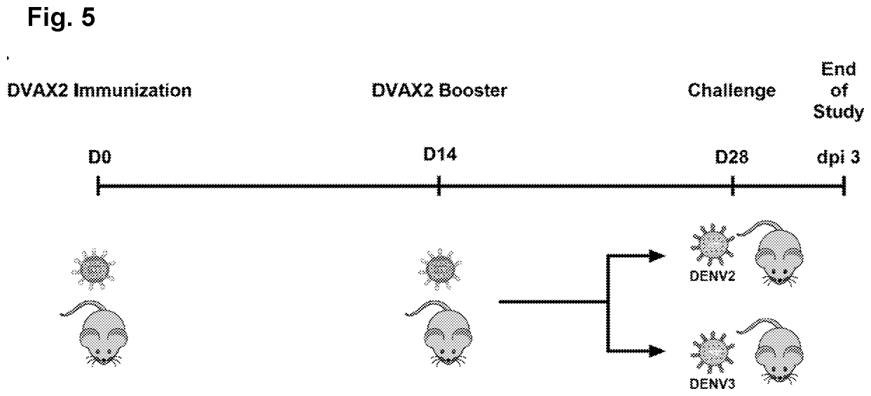

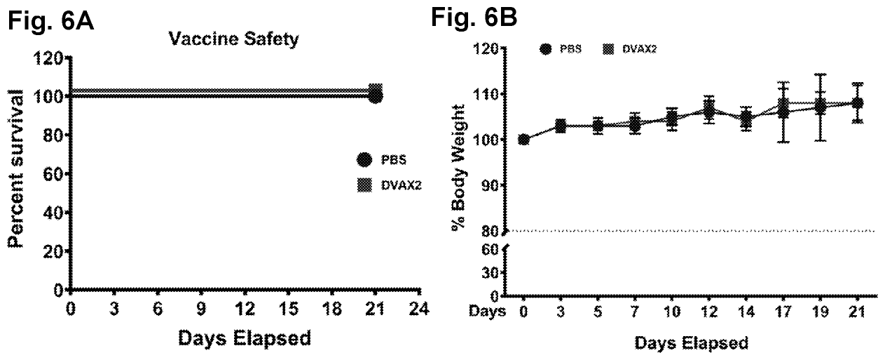

A schematic representation of this immunization study timeline is illustrated in FIG. 5. After producing vaccine particles, the safety of the DVAX vaccine was evaluated in adult Ifnar1−/− mice (mixed sex; 14 to 18 weeks old; n=4 to 5 animals/group). Mice were immunized with DVAX (1×10⁴ transduction units/mouse; subcutaneous route), and the unvaccinated group received phosphate-buffered saline (PBS) injection. The DVAX vaccine candidate is a replication-deficient subunit vaccine, therefore an additional booster dose was given 14 days after the primary immunization (1×10⁴ vaccine particle transduction units/mouse; by subcutaneous route) to enhance the DENV-specific immune response. After tracking survival over 21 days, it was found that mice vaccinated with DVAX had experienced no mortality. Vaccinated mice were healthy and viable until the day 21 endpoint (FIG. 6A). In addition, all vaccinated mice maintained normal body weight similar to the PBS group, thereby demonstrating that DVAX is well tolerated (FIG. 6B).

### DVAX is Effective in Preventing Wild-Type DENV Infection in Adult Mice.

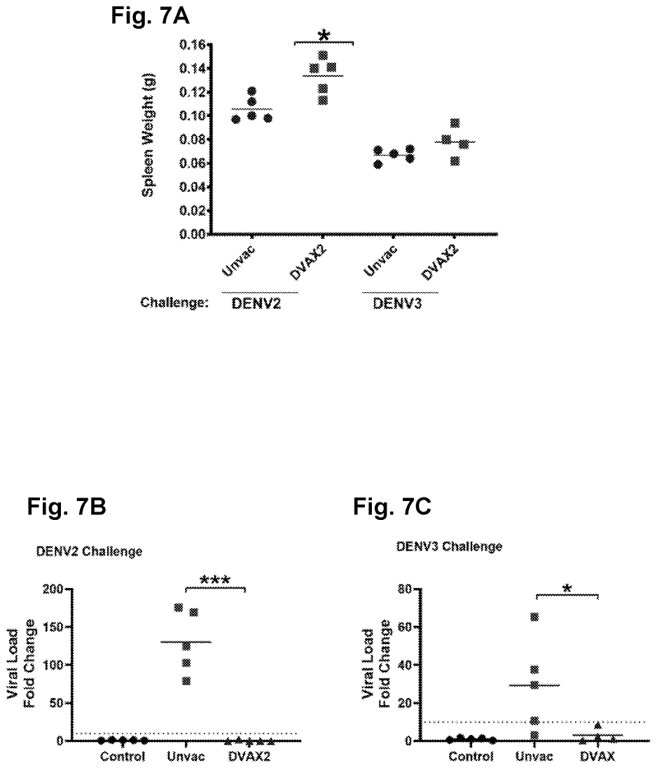

For proof of concept, the immunizations were done using a DVAX2 based on DENV serotype 2. At 2-weeks post-booster immunization, both the vaccinated and unvaccinated groups were challenged with wild-type DENV serotypes 2 or 3, and were followed for 3 days. DENV serotype 3 was used to assess potential ADE response in challenge studies as well as to define beneficial cross-protection. Mice vaccinated with DVAX2 effectively prevented the infection of WT DENV serotypes 2 and 3 challenges (FIGS. 7A-7C). Spleen weights of vaccinated animals show an increase in weight compared to unvaccinated challenged animals (FIG. 7A). Moreover, vaccination prevented viral replication in the spleen at 3 days post-challenge of WT DENV serotypes 2 and 3 compared to unvaccinated mice (FIGS. 7B-7C). These results indicate that the DVAX2 construct is safe and is effective in inducing a protective antiviral immune response against DENV infection. Moreover, the DVAX2 immunization followed by heterologous serotype 3 challenge did not show enhanced virus replication demonstrating cross-protection without evidence of the ADE phenomenon.

### EXAMPLES OF EMBODIMENTS

Example 1. A viral vector system, comprising:
one or more viral vector types, each viral vector type comprising:
   an outer layer comprising structural proteins of a non-dengue Orthoflavivirus species; and
   a nucleic acid for expressing one or more nonstructural proteins of dengue virus.

Example 2. The viral vector system of example 1, wherein the non-dengue Orthoflavivirus species is Japanese encephalitis virus, Tick-borne encephalitis virus, West Nile virus, Powassan virus, Yellow fever virus, or Zika virus.

Example 3. The viral vector system of example 2, wherein the non-dengue Orthoflavivirus species is Zika virus.

Example 4. The viral vector system of any one of examples 1-3, wherein the structural proteins comprise one or more of: C protein, E protein, and M protein.

Example 5. The viral vector system of example 4, wherein the structure proteins comprise C protein, E protein, and M protein.

Example 6. The viral vector of any one of examples 1-5, wherein the one or more nonstructural proteins comprise one or more of: NS1, NS2A, NS2B, NS3, NS4A, NS4B, and NS5.

Example 7. The viral vector system of any one of examples 1-6, wherein the one or more nonstructural proteins comprise NS1, NS2A, NS2B, NS3, NS4A, NS4B, and NS5.

Example 8. The viral vector system of any one of examples 1-7, wherein the nucleic acid is a positive-strand RNA.

Example 9. The viral vector system of any one of examples 1-8, wherein the nucleic acid comprises a sequence encoding the one or more nonstructural proteins of dengue virus that is at least 50% identical to its native sequence.

Example 10. The viral vector system of example 9, wherein the nucleic acid comprises a sequence encoding the one or more nonstructural proteins of dengue virus that is at least 70% identical to its native sequence.

Example 11. The viral vector system of any one of examples 1-10, wherein the viral vector system comprises a multivalent viral vector system comprises two or more genotypes, each genotype is of a unique dengue virus serotype.

Example 12. The viral vector system of example 11, wherein the viral vector system comprises the multivalent viral vector system comprises three or more genotypes, each genotype is of a unique dengue virus serotype.

Example 13. The viral vector system of any one of examples 11 or 12, wherein the viral vector system comprises the multivalent viral vector system comprises four or more genotypes, each genotype is of a unique dengue virus serotype.

Example 14. The viral vector system of any one of examples 11-13, wherein the viral vector system comprises two or more genotypes, each genotype is of a unique subtype of a serotype of dengue virus.

Example 15. The viral vector system of any one of examples 1-14, wherein the viral vector system is formulated for vaccine administration.

Example 16. A method of yielding viral vector products, comprising:
contacting a culture of packaging cells with:
   a nucleic acid comprising a first set of one or more expression constructs that encodes for one or more nonstructural proteins of dengue virus with a 5′-UTR a 3′-UTR, and a packaging signal, wherein the one or more nonstructural proteins are derived from native sequences of dengue virus; and
   a nucleic acid comprising a second set of one or more expression constructs that encodes for one or more structural proteins of a non-dengue Orthoflavivirus species, wherein the one or more structural proteins are derived from a native sequence of non-dengue Orthoflavivirus species.

Example 17. The method of example 16 further comprising:
preparing the viral vector products for vaccine administration.

Example 18. The method of any one of examples 16 or 17, wherein the non-dengue Orthoflavivirus species is Japanese encephalitis virus, Tick-borne encephalitis virus, West Nile virus, Powassan virus, Yellow fever virus, or Zika virus.

Example 19. The method of example 18, wherein the non-dengue Orthoflavivirus species is Zika virus.

Example 20. The method of any one of examples 16-19, wherein the structural proteins comprise one or more of: C protein, E protein, and M protein, or a precursor protein thereof.

Example 21. The method of example 20, wherein the structural proteins comprise: C protein, E protein, and M protein; or a precursor protein thereof.

Example 22. The method of any one of examples 16-21, wherein the one or more nonstructural proteins comprise one or more of: NS1, NS2A, NS2B, NS3, NS4A, NS4B, and NS5.

Example 23. The method of any one of examples 16-22, wherein the one or more nonstructural proteins comprise NS1, NS2A, NS2B, NS3, NS4A, NS4B, and NS5.

Example 24. The method of any one of examples 16-23, wherein the viral vector products comprise:
an outer layer comprising the one or more structural proteins of the non-dengue Orthoflavivirus species nucleic acid; and
a positive-strand RNA comprising sequences of the 5′-UTR, the 3′-UTR, packaging signal, and a genetic code for the one or more nonstructural proteins of the dengue virus.

Example 25. The method of example 24, wherein the genetic code for the one or more nonstructural proteins of the dengue virus is at least 50% identical to its native sequence.

Example 26. The method of example 25, wherein the genetic code for the one or more nonstructural proteins of the dengue virus is at least 70% identical to its native sequence.

Example 27. The method of any one of examples 16-26, wherein the sequence that encodes for one or more nonstructural proteins of dengue virus of the first set of one or more expression constructs is at least 50% identical to its native sequence.

Example 28. The method of example 27, wherein a genetic sequence that encodes for one or more nonstructural proteins of dengue virus of the first set of one or more expression constructs is at least 70% identical to its native sequence.

Example 29. The method of any one of examples 16-28, wherein a genetic sequence that encodes for one or more structural proteins of a non-dengue Orthoflavivirus species of the second set of one or more expression constructs is at least 50% identical to its native sequence.

Example 30. The method of example 29, wherein a genetic sequence that encodes for one or more structural proteins of a non-dengue Orthoflavivirus species of the second set of one or more expression constructs is at least 70% identical to its native sequence.

Example 31. The method of any one of examples 16-30, wherein the viral vector products comprise two or more genotypes, each genotype is of a unique dengue virus serotype.

Example 32. The method of any one of examples 16-31 further comprising:
storing product viral vectors lyophilized or in suspension.

Example 33. A method of vaccination for dengue virus, comprising:
administering a viral vector vaccine to a recipient, wherein the viral vector vaccine comprises:
   one or more viral vector types, each viral vector type comprising:
      an outer layer comprising structural proteins of a non-dengue Orthoflavivirus species; and
      a nucleic acid for expressing one or more nonstructural proteins of dengue virus.

Example 34. The method of example 33, wherein administering to the recipient the viral-vector based vaccine is in accordance with a vaccine schedule.

Example 35. The method of example 34, wherein the vaccine schedule comprises two or more administrations.

Example 36. The method of example 35, wherein the two or more administrations comprises a first administration and a second administration, wherein the first administration comprises a first set of one or more genotypes of dengue virus and the second administration comprises a second set of one or more genotypes of dengue virus.

Example 37. The method of example 36, wherein the first set of one or more genotypes of dengue virus genotypes is different than the second set of one or more genotypes of dengue virus genotypes.

Example 38. The method of example 36, wherein the first set of one or more genotypes of dengue virus genotypes is the same as the second set of one or more genotypes of dengue virus genotypes.

Example 39. The method of example 37, wherein the first set of one or more genotypes comprises a genotype of a first dengue virus serotype and the second set of one or more genotypes comprises a genotype of a second dengue virus serotype, wherein the first dengue virus serotype is different from the second dengue virus serotype.

Example 40. The method of example 37, wherein the first set of one or more genotypes comprises a genotype of a first subtype of a dengue virus serotype and the second set of one or more genotypes comprises a second subtype of the dengue virus serotype, wherein the first subtype of the dengue virus serotype is different from the second subtype of the dengue virus serotype.

Example 41. The method of example 37, wherein the first set of one or more genotypes comprises a genotype of a first subtype of a first dengue virus serotype and the second set of one or more genotypes comprises a second subtype of a second dengue virus serotype, wherein the first dengue virus serotype is different from the second dengue virus serotype.

Example 42. The method of any one of examples 33-41, wherein administering to the recipient with the viral-vector based vaccine is a first administration to yield an initial immune response.

Example 43. The method of any one of examples 33-41, wherein administering to the recipient the viral-vector based vaccine is a booster administration to boost an immune response.

Example 44. The method of any one of examples 33-43, wherein the recipient has been exposed to a first genotype of dengue virus, wherein a sequence encoding one or more nonstructural proteins of dengue virus within the nucleic acid is derived from a second genotype that is different from the first genotype.

Example 45. The method of example 44, wherein the recipient had a previous infection of dengue virus of the first genotype.

Example 46. The method of example 44, wherein the recipient had received a prior administration of a vaccine for dengue virus of the first genotype.

Example 47. The method of any one of examples 33-46, wherein the non-dengue Orthoflavivirus species is Japanese encephalitis virus, Tick-borne encephalitis virus, West Nile virus, Powassan virus, Yellow fever virus, or Zika virus.

Example 48. The method of example 47, wherein the non-dengue Orthoflavivirus species is Zika virus.

Example 49. The method of any one of examples 33-48, wherein the structural proteins comprise one or more of: C protein, E protein, and M protein.

Example 50. The method of example 49, wherein the structure proteins comprise C protein, E protein, and M protein.

Example 51. The method of any one of examples 33-50, wherein the one or more nonstructural proteins of dengue virus comprise one or more of: NS1, NS2A, NS2B, NS3, NS4A, NS4B, and NS5.

Example 52. The method of any one of examples 33-51, wherein the one or more nonstructural proteins of dengue virus comprise NS1, NS2A, NS2B, NS3, NS4A, NS4B, and NS5.

Example 53. The method any one of examples 33-52, wherein the nucleic acid comprises a sequence encoding the one or more nonstructural proteins of dengue virus that is at least 50% identical to its native sequence.

Example 54. The method of example 53, wherein the nucleic acid comprises a sequence encoding the one or more nonstructural proteins of dengue virus that is at least 70% identical to its native sequence.

Example 55. The method of any one of examples 33-54, wherein the viral vector vaccine is a multivalent viral vector vaccine comprising two or more genotypes of dengue virus.

Example 56. A viral vector system, comprising:
one or more viral vector types, each viral vector type comprising:
   an outer layer comprising structural proteins of Zika virus; and
   a nucleic acid for expressing one or more nonstructural proteins of a non-Zika Orthoflavivirus species.

Example 57. The viral vector system of example 56, wherein the non-Zika Orthoflavivirus species is Japanese encephalitis virus, Tick-borne encephalitis virus, West Nile virus, Powassan virus, Yellow fever virus, or dengue virus.

Example 58. The viral vector system of example 56 or 57, wherein the structural proteins comprise one or more of: C protein, E protein, and M protein.

Example 59. The viral vector system of example 58, wherein the structure proteins comprise C protein, E protein, and M protein.

Example 60. The viral vector system of any one of examples 56-59, wherein the one or more nonstructural proteins comprise one or more of: NS1, NS2A, NS2B, NS3, NS4A, NS4B, and NS5.

Example 61. The viral vector system of any one of examples 56-60, wherein the one or more nonstructural proteins comprise NS1, NS2A, NS2B, NS3, NS4A, NS4B, and NS5.

Example 62. The viral vector system of any one of examples 56-61, wherein the nucleic acid is a positive-strand RNA.

Example 63. The viral vector system of any one of examples 56-62, wherein the nucleic acid comprises a sequence encoding the one or more nonstructural proteins of the non-Zika Orthoflavivirus species that is at least 50% identical to its native sequence.

Example 64. The viral vector system of example 63, wherein the nucleic acid comprises a sequence encoding the one or more nonstructural proteins of the non-Zika Orthoflavivirus species that is at least 70% identical to its native sequence.

Example 65. The viral vector system of any one of examples 56-64, wherein the viral vector system comprises a multivalent viral vector system comprises two or more serotypes of the non-Zika Orthoflavivirus species.

Example 66. The viral vector system of any one of examples 56-65, wherein the viral vector system is formulated for vaccine administration.

TABLE 1
Sequence Lising Description

| SEQ ID | | |
|---|---|---|
| NO: | Sequence Name | Sequence Description |
| 1 | DV-1 NS1-NS5 NA | Dengue Virus Serotype 1 Nonstructural Proteins 1-5, Nucleic Acid |
| 2 | DV-2 NS1-NS5 NA | Dengue Virus Serotype 2 Nonstructural Proteins 1-5, Nucleic Acid |
| 3 | DV-3 NS1-NS5 NA | Dengue Virus Serotype 3 Nonstructural Proteins 1-5, Nucleic Acid |
| 4 | DV-4 NS1-NS5 NA | Dengue Virus Serotype 4 Nonstructural Proteins 1-5, Nucleic Acid |
| 5 | DV-1 5′UTR | Dengue Virus Serotype 1 5′-UTR |
| 6 | DV-2 5′UTR | Dengue Virus Serotype 2 5′-UTR |
| 7 | DV-3 5′UTR | Dengue Virus Serotype 3 5′-UTR |
| 8 | DV-4 5′UTR | Dengue Virus Serotype 4 5′-UTR |
| 9 | DV-1 3′UTR | Dengue Virus Serotype 1 3′-UTR |
| 10 | DV-2 3′UTR | Dengue Virus Serotype 2 3′-UTR |
| 11 | DV-3 3′UTR | Dengue Virus Serotype 3 3′-UTR |
| 12 | DV-4 3′UTR | Dengue Virus Serotype 4 3′-UTR |
| 13 | DV-1 partial C NA | Dengue Virus Serotype 1 partial Structural Protein C, Nucleic Acid |
| 14 | DV-2 partial C NA | Dengue Virus Serotype 2 partial Structural Protein C, Nucleic Acid |
| 15 | DV-3 partial C NA | Dengue Virus Serotype 3 partial Structural Protein C, Nucleic Acid |
| 16 | DV-4 partial C NA | Dengue Virus Serotype 4 partial Structural Protein C, Nucleic Acid |
| 17 | DV-1 partial E NA | Dengue Virus Serotype 1 partial Structural Protein E, Nucleic Acid |
| 18 | DV-2 partial E NA | Dengue Virus Serotype 2 partial Structural Protein E, Nucleic Acid |
| 19 | DV-3 partial E NA | Dengue Virus Serotype 3 partial Structural Protein E, Nucleic Acid |
| 20 | DV-4 partial E NA | Dengue Virus Serotype 4 partial Structural Protein E, Nucleic Acid |
| 21 | HDV ribozyme | Hepatitus Delta Virus Ribozyme Translational Enhancer |
| 22 | DV-1 NS1-NS5 AA | Dengue Virus Serotype 1 Nonstructural Proteins 1-5, Amino Acid |
| 23 | DV-2 NS1-NS5 AA | Dengue Virus Serotype 2 Nonstructural Proteins 1-5, Amino Acid |
| 24 | DV-3 NS1-NS5 AA | Dengue Virus Serotype 3 Nonstructural Proteins 1-5, Amino Acid |
| 25 | DV-4 NS1-NS5 AA | Dengue Virus Serotype 4 Nonstructural Proteins 1-5, Amino Acid |
| 26 | DV-1 partial C AA | Dengue Virus Serotype 1 partial Structural Protein C, Amino Acid |
| 27 | DV-2 partial C AA | Dengue Virus Serotype 2 partial Structural Protein C, Amino Acid |
| 28 | DV-3 partial C AA | Dengue Virus Serotype 3 partial Structural Protein C, Amino Acid |
| 29 | DV-4 partial C AA | Dengue Virus Serotype 4 partial Structural Protein C, Amino Acid |
| 30 | DV-1 partial E AA | Dengue Virus Serotype 1 partial Structural Protein E, Amino Acid |
| 31 | DV-2 partial E AA | Dengue Virus Serotype 2 partial Structural Protein E, Amino Acid |
| 32 | DV-3 partial E AA | Dengue Virus Serotype 3 partial Structural Protein E, Amino Acid |
| 33 | DV-4 partial E AA | Dengue Virus Serotype 4 partial Structural Protein E, Amino Acid |
| 34 | ZV CME NA | Zika Virus Structural Proteins CME, Nucleic Acid |
| 35 | ZV CME AA | Zika Virus Structural Proteins CME, Amino Acid |
| 35 | ZV C AA | Zika Virus Structural Protein C, Amino Acid |
| 35 | ZV M AA | Zika Virus Structural Protein M, Amino Acid |
| 38 | ZV E AA | Zika Virus Structural Protein E, Amino Acid |

TABLE 2
Amino Acid Sequences

| SEQ | Sequence | |
|---|---|---|
| ID NO: | Name | Sequence |
| 1 | DV-1 | GACTCGGGAT GTGTAATCAA CTGGAAAGGC AGAGAACTCA AATGTGGAAG |
| | NS1-NS5 | CGGCATTTTT GTCACCAATG AAGTCCACAC CTGGACAGAG CAATATAAAT |
| | NA | TCCAGGCCGA CTCCCCTAAG AGACTATCAG CGGCCATTGG GAAGGCATGG |
| | | GAGGAGGGTG TGTGTGGAAT TCGATCAGCC ACTCGTCTCG AGAACATCAT |
| | | GTGGAAGCAA ATATCAAATG AATTAAACCA CATCTTACTT GAAAATGACA |
| | | TGAAATTTAC AGTGGTCGTA GGAGACGTTA GTGGAATCTT GGCCCAAGGA |
| | | AAGAAAATGA TTAGGCCACA ACCCATGGAA CACAAATACT CGTGGAAAAG |
| | | CTGGGGAAAA GCCAAAATCA TAGGAGCAGA TGTACAGAAT ACCACCTTCA |
| | | TCATCGACGG CCCAAACACC CCAGAATGCC CTGATAACCA AAGAGCATGG |
| | | AACATTTGGG AAGTTGAAGA CTATGGATTT GGAATTTTCA CGACAAACAT |
| | | ATGGTTGAAA TTGCGTGACT CCTACACTCA AGTGTGTGAC CACCGGCTAA |
| | | TGTCAGCTGC CATCAAGGAT AGCAAAGCAG TCCATGCTGA CATGGGGTAC |
| | | TGGATAGAAA GTGAAAAGAA CGAGACTTGG AAGTTGGCAA GAGCCTCCTT |
| | | CATAGAAGTT AAGACATGCA TCTGGCCAAA ATCCCACACT CTATGGAGCA |
| | | ATGGAGTCCT GGAAAGTGAG ATGATAATCC CAAAGATATA TGGAGGACCA |
| | | ATATCTCAGC ACAACTACAG ACCAGGATAT TTCACACAAA CAGCAGGGCC |
| | | GTGGCACTTG GGCAAGTTAG AACTAGATTT TGATTTATGT GAAGGTACCA |
| | | CTGTTGTTGT GGATGAACAT TGTGGAAATC GAGGACCATC TCTTAGAACC |
| | | ACAACAGTCA CAGGAAAGAC AATCCATGAA TGGTGCTGTA GATCTTGCAC |
| | | GTTACCCCCC CTACGTTTCA AAGGAGAAGA CGGGTGCTGG TACGGCATGG |
| | | AAATCAGACC AGTCAAGGAG AAGGAAGAGA ACCTAGTTAA GTCAATGGTC |
| | | TCTGCAGGGT CAGGAGAAGT GGACAGTTTT TCACTAGGAC TGCTATGCAT |
| | | ATCAATAATG ATCGAAGAGG TAATGAGATC CAGATGGAGC AGAAAAATGC |
| | | TGATGACTGG AACATTGGCT GTGTTCCTCC TTCTCACAAT GGGACAATTG |
| | | ACATGGAATG ATCTGATCAG GCTATGTATC ATGGTTGGAG CCAACGCTTC |
| | | AGACAAGATG GGGATGGGAA CAACGTACCT AGCTTTGATG GCCACTTTCA |
| | | GAATGAGACC AATGTTCGCA GTCGGGCTAC TGTTTCGCAG ATTAACATCT |
| | | AGAGAAGTTC TTCTTCTTAC AGTTGGATTG AGTCTGGTGG CATCTGTAGA |
| | | ACTACCAAAT TCCTTAGAGG AGCTAGGGGA TGGACTTGCA ATGGGCATCA |
| | | TGATGTTGAA ATTACTGACT GATTTTCAGT CACATCAGCT ATGGGCTACC |
| | | TTGCTGTCTT TAACATTTGT CAAAACAACT TTTTCATTGC ACTATGCATG |
| | | GAAGACAATG GCTATGATAC TGTCAATTGT ATCTCTCTTC CCTTTATGCC |
| | | TGTCCACGAC TTCTCAAAAA ACAACATGGC TTCCGGTGTT GCTGGGATCT |
| | | CTTGGATGCA AACCACTAAC CATGTTTCTT ATAACAGAAA ACAAAATCTG |
| | | GGGAAGGAAA AGCTGGCCTC TCAATGAAGG AATTATGGCT GTTGGAATAG |
| | | TTAGCATTCT TCTAAGTTCA CTTCTCAAGA ATGATGTGCC ACTAGCTGGC |
| | | CCACTAATAG CTGGAGGCAT GCTAATAGCA TGTTATGTCA TATCTGGAAG |
| | | CTCGGCCGAT TTATCACTGG AGAAAGCGGC TGAGGTCTCC TGGGAAGAAG |
| | | AAGCAGAACA CTCTGGTGCC TCACACAACA TACTAGTGGA GGTCCAAGAT |
| | | GATGGAACCA TGAAGATAAA GGATGAAGAG AGAGATGACA CACTCACCAT |
| | | TCTCCTCAAA GCAACTCTGC TAGCAATCTC AGGGGTATAC CCAATGTCAA |
| | | TACCGGCGAC CCTCTTTGTG TGGTATTTTT GGCAGAAAAA GAAACAGAGA |
| | | TCAGGAGTGC TATGGGACAC ACCCAGCCCT CCAGAAGTGG AAAGAGCAGT |
| | | CCTTGATGAT GGCATTTATA GAATTCTCCA AAGAGGATTG TTGGGCAGGT |
| | | CTCAAGTAGG AGTAGGAGTT TTTCAAGAAG GCGTGTTCCA CACAATGTGG |
| | | CACGTCACCA GGGGAGCTGT CCTCATGTAC CAAGGGAAGA GACTGGAACC |
| | | AAGTTGGGCC AGTGTCAAAA AAGACTTGAT CTCATATGGA GGAGGTTGGA |
| | | GGTTTCAAGG ATCCTGGAAC GCGGGAGAAG AAGTGCAGGT GATTGCTGTT |
| | | GAACCGGGGA AGAACCCCAA AAATGTACAG ACAGCGCCGG GTACCTTCAA |
| | | GACCCCTGAA GGCGAAGTTG GAGCCATAGC TCTAGACTTT AAACCCGGCA |
| | | CATCTGGATC TCCTATCGTG AACAGAGAGG GAAAAATAGT AGGTCTTTAT |
| | | GGAAATGGAG TGGTGACAAC AAGTGGTACC TACGTCAGTG CCATAGCTCA |
| | | AGCTAAAGCA TCACAAGAAG GGCCTCTACC AGAGATTGAG GACGAGGTGT |
| | | TTAGGAAAAG AAACTTAACA ATAATGGACC TACATCCAGG ATCGGGAAAA |
| | | ACAAGAAGAT ACCTTCCAGC CATAGTCCGT GAGGCCATAA AAAGAAAGCT |
| | | GCGCACGCTA GTCTTAGCTC CCACAAGAGT TGTCGCTTCT GAAATGGCAG |
| | | AGGCGCTCAA GGGAATGCCA ATAAGGTATC AGACAACAGC AGTGAAGAGT |
| | | GAACACACGG GAAAGGAGAT AGTTGACCTT ATGTGTCACG CCACTTTCAC |
| | | TATGCGTCTC CTGTCTCCTG TGAGAGTTCC CAATTATAAT ATGATTATCA |
| | | TGGATGAAGC ACATTTTACC GATCCAGCCA GCATAGCAGC CAGAGGGTAT |
| | | ATCTCAACCC GAGTGGGTAT GGGTGAAGCA GCTGCGATTT TCATGACAGC |
| | | CACTCCCCCC GGATCGGTGG AGGCCTTTCC ACAGAGCAAT GCAGTTATCC |
| | | AAGATGAGGA AAGAGACATT CCTGAAAGAT CATGGAACTC AGGCTATGAC |
| | | TGGATCACTG ATTTCCCAGG TAAAACAGTC TGGTTTGTTC CAAGCATCAA |
| | | ATCAGGAAAT GACATTGCCA ACTGTTTAAG AAAGAATGGG AAACGGGTGG |
| | | TCCAATTGAG CAGAAAAACT TTTGACACTG AGTACCAGAA AACAAAAAAT |
| | | AACGACTGGG ACTATGTTGT CACAACAGAC ATATCCGAAA TGGGAGCAAA |
| | | CTTCCGAGCC GACAGGGTAA TAGACCCGAG GCGGTGCCTG AAACCGGTAA |
| | | TACTAAAAGA TGGCCCAGAG CGTGTCATTC TAGCCGGACC GATGCCAGTG |
| | | ACTGTGGCTA GCGCCGCCCA GAGGAGAGGA AGAATTGGAA GGAACCAAAA |
| | | TAAGGAAGGC GATCAGTATA TTTACATGGG ACAGCCTCTA AACAATGATG |
| | | AGGACCACGC CCATTGGACA GAAGCAAAAA TGCTCCTTGA CAACATAAAC |
| | | ACACCAGAAG GGATTATCCC AGCCCTCTTT GAGCCGGAGA GAGAAAAGAG |
| | | TGCAGCAATA GACGGGGAAT ACAGACTACG GGGTGAAGCG AGGAAAACGT |
| | | TCGTGGAGCT CATGAGAAGA GGAGATCTAC CTGTCTGGCT ATCCTACAAA |
| | | GTTGCCTCAG AAGGCTTCCA GTACTCCGAC AGAAGGTGGT GCTTTGATGG |
| | | GGAAAGGAAC AACCAGGTGT TGGAGGAGAA CATGGACGTG GAGATCTGGA |
| | | CAAAAGAAGG AGAAAGAAAG AAACTACGAC CCCGCTGGCT GGATGCCAGA |
| | | ACATACTCTG ACCCACTGGC TCTGCGCGAA TTCAAAGAGT TCGCAGCAGG |
| | | AAGAAGAAGC GTCTCAGGTG ACCTAATATT AGAAATAGGG AAACTTCCAC |
| | | AACATTTAAC GCAAAGGGCC CAGAACGCCT TGGACAATCT GGTTATGTTG |
| | | CACAACTCTG AACAAGGAGG AAAAGCCTAT AGACACGCCA TGGAAGAACT |
| | | ACCAGACACC ATAGAAACGT TAATGCTCCT AGCTTTGATA GCTGTGCTGA |
| | | CTGGTGGAGT GACGTTGTTC TTCCTATCAG GAAGGGGTCT AGGAAAAACA |
| | | TCCATTGGCC TACTCTGCGT GATTGCCTCA AGTGCACTGT TATGGATGGC |
| | | CAGTGTGGAA CCCCATTGGA TAGCGGCCTC TATCATACTG GAGTTCTTTC |
| | | TGATGGTGTT GCTTATTCCA GAGCCGGACA GACAGCGCAC TCCACAAGAC |
| | | AACCAGCTAG CATACGTGGT GATAGGTCTG TTATTCATGA TATTGACAGT |
| | | GGCAGCCAAT GAGATGGGAT TACTGGAAAC CACAAAGAAG GACCTGGGGA |
| | | TTGGTCATGC AGCTGCTGAA AACCACCATC ATGCTGCAAT GCTGGACGTA |
| | | GACCTACATC CAGCTTCAGC CTGGACTCTC TATGCAGTGG CCACAACAAT |
| | | TATCACTCCC ATGATGAGAC ACACAATTGA AAACACAACG GCAAATATTT |
| | | CCCTGACAGC TATTGCAAAC CAGGCAGCTA TATTGATGGG ACTTGACAAG |
| | | GGATGGCCAA TATCAAAGAT GGACATAGGA GTTCCACTTC TCGCCTTGGG |
| | | GTGCTATTCT CAGGTGAACC CGCTGACGCT GACAGCGGCG GTATTGATGC |
| | | TAGTGGCTCA TTATGCCATA ATTGGACCCG GACTGCAAGC AAAAGCTACT |
| | | AGAGAAGCTC AAAAAAGGAC AGCAGCCGGA ATAATGAAAA ACCCAACTGT |
| | | CGACGGGATC GTTGCAATAG ATTTGGACCC TGTGGTTTAC GATGCAAAAT |
| | | TTGAAAAACA GCTAGGCCAA ATAATGTTGT TGATACTTTG CACATCACAG |
| | | ATCCTCCTGA TGCGGACCAC ATGGGCCTTG TGTGAATCCA TCACACTAGC |
| | | CACTGGACCT CTGACTACGC TTTGGGAGGG ATCTCCAGGA AAATTCTGGA |
| | | ACACCACGAT AGCGGTGTCC ATGGCAAACA TTTTTAGGGG AAGTTATCTA |
| | | GCAGGAGCAG GTCTGGCCTT TTCATTAATG AAATCTCTAG GAGGAGGTAG |
| | | GAGAGGCACG GGAGCCCAAG GGGAAACACT GGGAGAAAAA TGGAAAAGAC |
| | | AGCTAAACCA ATTGAGCAAG TCAGAATTCA ACACTTACAA AAGGAGTGGG |
| | | ATTATAGAGG TGGATAGATC TGAAGCCAAA GAGGGGTTAA AAAGAGGAGA |
| | | AACGACTAAA CACGCAGTGT CGAGAGGAAC GGCCAAACTG AGGTGGTTTG |
| | | TGGAGAGGAA CCTTGTGAAA CCAGAAGGGA AAGTCATAGA CCTCGGTTGT |
| | | GGAAGAGGTG GCTGGTCATA TTATTGCGCT GGGCTGAAGA AAGTCACAGA |
| | | AGTGAAAGGA TACACGAAAG GAGGACCTGG ACATGAGGAA CCAATCCCAA |
| | | TGGCAACCTA TGGATGGAAC CTAGTAAAGC TATACTCCGG GAAAGATGTA |
| | | TTCTTTACAC CACCTGAGAA ATGTGACACC CTCTTGTGTG ATATTGGTGA |
| | | GTCCTCTCCG AACCCAACTA TAGAAGAAGG AAGAACGTTA CGTGTTCTAA |
| | | AGATGGTGGA ACCATGGCTC AGAGGAAACC AATTTTGCAT AAAAATTCTA |
| | | AATCCCTATA TGCCGAGTGT GGTAGAAACT TTGGAGCAAA TGCAAAGAAA |
| | | ACATGGAGGA ATGCTAGTGC GAAATCCACT CTCAAGAAAC TCCACTCATG |
| | | AAATGTACTG GGTTTCATGT GGAACAGGAA ACATTGTGTC AGCAGTAAAC |
| | | ATGACATCTA GAATGCTGCT AAATCGATTC ACAATGGCTC ACAGGAAGCC |
| | | AACATATGAA AGAGACGTGG ACTTAGGCGC TGGAACAAGA CATGTGGCAG |
| | | TAGAACCAGA GGTGGCCAAC CTAGATATCA TTGGCCAGAG GATAGAGAAT |
| | | ATAAAAAATG AACACAAATC AACATGGCAT TATGATGAGG ACAATCCATA |
| | | CAAAACATGG GCCTATCATG GATCATATGA GGTCAAGCCA TCAGGATCAG |
| | | CCTCATCCAT GGTCAATGGT GTGGTGAGAC TGCTAACCAA ACCATGGGAT |
| | | GTCATTCCCA TGGTCACACA AATAGCCATG ACTGACACCA CACCCTTTGG |
| | | ACAACAGAGG GTGTTTAAAG AGAAAGTTGA CACGCGTACA CCAAAAGCGA |
| | | AACGAGGCAC AGCACAAATT ATGGAGGTGA CAGCCAGGTG GTTATGGGGT |
| | | TTTCTCTCTA GAAACAAAAA ACCCAGAATC TGCACAAGAG AGGAGTTCAC |
| | | AAGAAAAGTC AGGTCAAACG CAGCTATTGG AGCAGTGTTC GTTGATGAAA |
| | | ATCAATGGAA CTCAGCAAAA GAGGCAGTGG AAGATGAACG GTTCTGGGAC |
| | | CTTGTGCACA GAGAGAGGGA GCTTCATAAA CAAGGAAAAT GTGCCACGTG |
| | | TGTCTACAAC ATGATGGGAA AGAGAGAGAA AAAATTAGGA GAGTTCGGAA |
| | | AGGCAAAAGG AAGTCGCGCA ATATGGTACA TGTGGTTGGG AGCGCGCTTT |
| | | TTAGAGTTTG AAGCCCTTGG TTTCATGAAT GAAGATCACT GGTTCAGCAG |
| | | AGAGAATTCA CTCAGTGGAG TGGAAGGAGA AGGACTCCAC AAACTTGGAT |
| | | ACATACTCAG AGACATATCA AAGATTCCAG GGGGAAATAT GTATGCAGAT |
| | | GACACAGCCG GATGGGACAC AAGAATAACA GAGGATGATC TTCAGAATGA |
| | | GGCCAAAATC ACTGACATCA TGGAACCTGA ACATGCCCTA TTGGCCACGT |
| | | CAATCTTTAA GCTAACCTAC CAAAACAAGG TAGTAAGGGT GCAGAGACCA |
| | | GCGAAAAATG GAACCGTGAT GGATGTCATA TCCAGACGTG ACCAGAGAGG |
| | | AAGTGGACAG GTTGGAACCT ATGGCTTAAA CACCTTCACC AACATGGAGG |
| | | CCCAACTAAT AAGACAAATG GAGTCTGAGG GAATCTTTTC ACCCAGCGAA |
| | | TTGGAAACCC CAAATCTAGC CGAAAGAGTC CTCGACTGGT TGAAAAAACA |
| | | TGGCACCGAG AGGCTGAAAA GAATGGCAAT CAGTGGAGAT GACTGTGTGG |
| | | TGAAACCAAT CGATGACAGA TTTGCAACAG CCTTAACAGC TTTGAATGAC |
| | | ATGGGAAAGG TAAGAAAAGA CATACCGCAA TGGGAACCTT CAAAAGGATG |
| | | GAATGATTGG CAACAAGTGC CTTTCTGTTC ACACCATTTC CACCAGCTGA |
| | | TTATGAAGGA TGGGAGGGAG ATAGTGGTGC CATGCCGCAA CCAAGATGAA |
| | | CTTGTAGGTA GGGCCAGAGT ATCACAAGGC GCCGGATGGA GCTTGAGAGA |
| | | AACTGCATGC CTAGGCAAGT CATATGCACA AATGTGGCAG CTGATGTACT |
| | | TCCACAGGAG AGACTTGAGA TTAGCGGCTA ATGCTATCTG TTCAGCCGTT |
| | | CCAGTTGATT GGGTCCCAAC CAGCCGCACC ACCTGGTCGA TCCATGCCCA |
| | | CCATCAATGG ATGACAACAG AAGACATGTT GTCAGTGTGG AATAGGGTTT |
| | | GGATAGAGGA AAACCCATGG ATGGAGGACA AGACTCATGT GTCCAGTTGG |
| | | GAAGACGTTC CATACCTAGG AAAAAGGGAA GATCAATGGT GTGGTTCCCT |
| | | AATAGGCTTA ACAGCACGAG CCACCTGGGC CACCAACATA CAAGTGGCCA |
| | | TAAACCAAGT GAGAAGGCTC ATTGGGAATG AGAATTATCT AGACTTCATG |
| | | ACATCAATGA AGAGATTCAA AAACGAGAGT GATCCCGAAG GGGCACTCTG |
| | | GTAA |
| 2 | DV-2 | GATAGTGGTT GCGTTGTGAG CTGGAAAAAC AAAGAACTGA AATGTGGCAG |
| | NS1-NS5 | TGGGATTTTC ATCACAGACA ACGTGCACAC ATGGACAGAA CAATACAAGT |
| | NA | TCCAACCAGA ATCCCCTTCA AAACTAGCTT CAGCTATCCA GAAAGCTCAA |
| | | GAAGAGGGAA TTTGTGGAAT CCGCTCAGTA ACAAGACTGG AGAATCTGAT |
| | | GTGGAAACAA ATAACACCAG AATTGAATCA CATTCTAGCA GAAAATGAGG |
| | | TGAAGTTAAC TATCATGACA GGAGACATCA AAGGAATCAT GCAGGCAGGA |
| | | AAACGATCTC TGCGGCCTCA GCCCACTGAG CTAAAGTATT CATGGAAAAC |
| | | ATGGGGCAAA GCAAAAATGC TCTCCACAGA GTCTCATAAC CAAACCTTTC |
| | | TCATTGATGG CCCCGAAACA GCAGAATGCC CCAACACAAA TAGAGCTTGG |
| | | AATTCGTTGG AAGTTGAAGA CTACGGCTTT GGAGTATTCA CCACCAATAT |
| | | ATGGCTAAAA TTGAAAGAAA AACAGGATGC ATTCTGCGAC TCAAAACTCA |
| | | TGTCAGCGGC CATAAAAGAC AATAGAGCCG TCCATGCCGA TATGGGTTAT |
| | | TGGATAGAAA GTGCACTCAA TGACACATGG AAGATAGAGA AAGCCTCTTT |
| | | CATTGAAGTT AAAAATTGCC ACTGGCCAAA ATCACACACC CTCTGGAGCA |
| | | ATGGAGTGCT AGAAAGTGAG ATGATAATTC CAAAGAATCT CGCTGGACCA |
| | | GTGTCTCAAC ACAACTATAG ACCAGGCTAC CATACACAAA TAGCAGGACC |
| | | ATGGCATCTA GGTAAGCTTG AGATGGACTT TGATTTCTGC GATGGAACTA |
| | | CAGTGGTAGT GACTGAGGAC TGCGGAAATA GAGGACCCTC TTTGAGAACA |
| | | ACCACTGCCT CTGGAAAACT CATAACAGAA TGGTGCTGCC GATCTTGCAC |
| | | ATTACCACCG CTAAGATACA GAGGTGAGGA TGGGTGCTGG TACGGGATGG |
| | | AAATCAGACC ATTGAAAGAG AAAGAAGAAA ATTTGGTTAA CTCCTTGGTC |
| | | ACAGCCGGAC ATGGGCAGGT TGACAACTTT TCACTAGGAG TCTTGGGAAT |
| | | GGCATTGTTC CTGGAGGAAA TGCTTAGGAC CCGAGTAGGA ACGAAACATG |
| | | CAATACTACT AGTTGCAGTT TCTTTTGTGA CATTGATCAC AGGGAACATG |
| | | TCCTTTAGAG ACCTAGGGAG AGTGGTGGTT ATGGTAGGCG CCACTATGAC |
| | | GGATGACATA GGTATGGGCG TGACTTATCT TGCCCTACTA GCAGCCTTCA |
| | | AAGTCAGACC AACTTTTGCA GCTGGACTAC TCTTGAGAAA GCTGACCTCC |
| | | AAGGAATTGA TGATGACTAC TATAGGAATT GTACTCCTCT CCCAAAGCAC |
| | | CATACCAGAG ACCATTCTTG AATTGACTGA TGCGTTAGCC TTGGGCATGA |
| | | TGGTCCTCAA AATGGTAAGA AATATGGAAA AGTATCAATT AGCAGTGACT |
| | | ATCATGGCTA TCTTGTGCGT CCCAAACGCA GTGATATTAC AAAACGCATG |
| | | GAAAGTGAGT TGCACAATAC TAGCAGTGGT GTCCGTTTCC CCATTGCTTT |
| | | TAACATCCTC ACAGCAAAAA ACGGATTGGA TACCATTAGC ATTGACAATC |
| | | AAGGGTCTCA ACCCAACAGC TATTTTTCTA ACAACCCTCT CAAGAACCAG |
| | | CAAGAAAAGG AGCTGGCCAT TAAATGAGGC TATCATGGCA GTCGGGATGG |
| | | TGAGCATTTT AGCCAGTTCT CTCCTAAAAA ATGATATTCC CATGACAGGA |
| | | CCACTAGTGG CTGGAGGGCT CCTCACTGTG TGCTACGTGC TCACTGGACG |
| | | ATCGGCCGAT TTGGAACTGG AGAGAGCAGC CGATGTCAAA TGGGAAGACC |
| | | AGGCAGAGAT ATCAGGAAGC AGTCCAATCC TGTCAATAAC AATATCAGAA |
| | | GATGGTAGCA TGTCAATAAA AAATGAAGAG GAAGAACAAA CACTGACCAT |
| | | ACTCATTAGA ACAGGATTGC TGGTGATCTC AGGACTTTTT CCTGTATCAA |
| | | TACCAATCAC GGCAGCAGCA TGGTACCTGT GGGAAGTGAA GAAACAACGG |
| | | GCCGGAGTAT TGTGGGATGT TCCTTCACCC CCACCCATGG GAAAGGCTGA |
| | | ACTGGAAGAT GGAGCCTATA GAATCAAGCA AAAAGGGATT CTTGGATATT |
| | | CCCAGATCGG AGCCGGAGTT TACAAAGAAG GAACATTCCA TACAATGTGG |
| | | CATGTCACAC GTGGCGCTGT CCTAATGCAC AAAGGAAAGA GGATTGAACC |
| | | ATCATGGGCG GACGTCAAGA AAGATCTAAT ATCATATGGA GGAGGCTGGA |
| | | AGTTAGAAGG AGAATGGAAG GAAGGAGAAG AAGTCCAGGT ATTGGCATTG |
| | | GAGCCTGGAA AAAATCCAAG AGCCGTCCAA ACAAAACCTG GTCTTTTCAG |
| | | AACCAACGCC GGAACAATAG GTGCCGTATC TCTGGACTTT TCTCCTGGAA |
| | | CGTCAGGATC TCCAATCATT GACAAAAAAG GAAAAGTTGT GGGTCTTTAT |
| | | GGTAATGGTG TTGTTACAAG GAGTGGAGCA TATGTGAGTG CTATAGCCCA |
| | | GACAGAAAAA AGCATTGAAG ACAACCCAGA GATCGAAGAT GACATTTTCC |
| | | GAAAGAGAAG ACTGACCATA CTGGACCTCC ATCCAGGAGC GGGAAAGACG |
| | | AAAAGATACC TTCCGGCCAT AGTCAGAGAA GCTATAAAAC GGGGTTTGAG |
| | | AACATTAATC TTGGCTCCCA CTAGAGTTGT GGCAGCTGAA ATGGAGGAAG |
| | | CTCTTAGAGG ACTTCCAATA AGATACCAAA CCCCAGCCAT CAGAGCTGAG |
| | | CACACCGGGC GGGAGATTGT GGACCTAATG TGTCATGCCA CATTTACCAT |
| | | GAGGCTGCTA TCACCAGTTA GAGTGCCAAA CTACAACCTG ATTATCATGG |
| | | ACGAAGCCCA TTTCACAGAC CCAGCAAGTA TAGCAGCCAG AGGATACATC |
| | | TCAACTCGAG TGGAGATGGG TGAGGCAGCT GGGATTTTCA TGACAGCCAC |
| | | TCCCCCGGGA AGCAGAGACC CATTTCCTCA GAGCAATGCA CCAATCATAG |
| | | ATGAAGAAAG AGAAATCCCT GAACGTTCGT GGAATTCTGG ACATGAGTGG |
| | | GTCACGGATT TCAAAGGGAA GACTGTTTGG TTCGTTCCAA GTATAAAAGC |
| | | AGGAAATGAT ATAGCAGCTT GCCTGAGAAA AAATGGAAAG AAAGTGATAC |
| | | AACTCAGTAG GAAGACTTTT GATTCTGAGT ATGTCAAGAC TAGAACCAAT |
| | | GATTGGGATT TCGTGGTTAC AACTGACATT TCAGAAATGG GTGCCAATTT |
| | | CAAGGCTGAG AGGGTTATAG ACCCCAGACG CTGCATGAAA CCAGTCATAC |
| | | TAACAGATGG TGAGGAGCGG GTGATTCTGG CAGGACCTAT GCCAGTGACC |
| | | CACTCTAGTG CAGCACAAAG AAGAGGGAGA ATAGGAAGAA ATCCAAAAAA |
| | | TGAAAATGAC CAGTACATAT ACATGGGGGA ACCTCTGGAA AATGATGAAG |
| | | ACTGTGCACA CTGGAAAGAA GCCAAAATGC TCCTAGATAA CATCAACACA |
| | | CCAGAAGGAA TCATCCCTAG CATGTTCGAA CCAGAGCGTG AAAAAGTGGA |
| | | TGCCATTGAT GGCGAATACC GCTTGAGAGG AGAAGCAAGG AAAACCTTTG |
| | | TAGACTTAAT GAGAAGAGGA GACCTACCAG TCTGGTTGGC CTACAAAGTG |
| | | GCAGCTGAAG GCATCAACTA CGCAGACAGA AGGTGGTGTT TTGATGGAAT |
| | | CAAGAACAAC CAAATCTTGG AAGAAAACGT GGAAGTTGAA ATCTGGACAA |
| | | AAGAAGGGGA AAGGAAGAAA TTGAAACCCA GATGGCTGGA TGCTAGGATC |
| | | TATTCTGACC CACTGGCGCT AAAAGAATTT AAGGAATTTG CAGCCGGAAG |
| | | AAAGTCTCTG ACCCTGAACC TAATCACAGA AATGGGTAGG CTTCCAACCT |
| | | TTATGACTCA GAAGGCAAGA GACGCACTGG ACAACTTAGC AGTGCTGCAT |
| | | ACGGCTGAAG CAGGTGGAAG GGCGTACAAT CATGCTCTCA GTGAACTGCC |
| | | GGAGACCCTG GAGACATTGC TTTTACTGAC ACTGCTGGCT ACAGTCACGG |
| | | GAGGGATCTT TTTATTCTTG ATGAGCGGGA GGGGCATAGG GAAGATGACC |
| | | CTGGGAATGT GCTGCATAAT CACGGCTAGC ATCCTCCTAT GGTACGCACA |
| | | AATACAGCCA CACTGGATAG CAGCTTCAAT AATACTGGAG TTTTTTCTCA |
| | | TAGTTTTGCT TATTCCAGAA CCAGAAAAAC AGAGAACACC CCAAGACAAC |
| | | CAATTGACCT ACGTTGTCAT AGCCATCCTC ACAGTGGTGG CCGCAACCAT |
| | | GGCAAACGAG ATGGGTTTCC TAGAAAAAAC GAAGAAAGAT CTTGGATTGG |
| | | GAAGCATTGC AACCCAGCAA CCCGAGAGCA ACATCCTGGA CATAGATCTA |
| | | CGTCCTGCAT CAGCATGGAC GCTGTATGCC GTGGCCACAA CATTTGTTAC |
| | | ACCAATGTTG AGACATAGCA TTGAAAATTC CTCAGTGAAT GTATCCCTAA |
| | | CAGCTATAGC CAACCAAGCC ACAGTGTTAA TGGGTCTCGG GAAAGGATGG |
| | | CCATTGTCAA AGATGGACAT CGGAGTTCCC CTTCTCGCCA TTGGATGCTA |
| | | CTCACAAGTC AATCCCATAA CTCTCACAGC AGCTCTTCTC TTATTGGTAG |
| | | CGCATTATGC CATCATAGGG CCAGGACTTC AAGCAAAAGC AACCAGGGAA |
| | | GCTCAGAAAA GAGCAGCGGC GGGCATCATG AAAAATCCAA CCGTCGATGG |
| | | AATAACAGTG ATTGACCTAG ATCCAATACC CTATGATCCA AAGTTTGAAA |
| | | AGCAGTTGGG ACAAGTAATG CTCCTAGTCC TCTGCGTGAC TCAAGTATTG |
| | | ATGATGAGGA CTACATGGGC TCTGTGTGAG GCTTTAACCT TAGCTACCGG |
| | | GCCCATCTCC ACACTGTGGG AAGGAAATCC AGGAAGATTT TGGAACACTA |
| | | CCATTGCGGT GTCAATGGCT AACATTTTTA GAGGGAGTTA CTTGGCTGGA |
| | | GCTGGACTTC TCTTTTCTAT TATGAAGAAC ACAACCAACA CAAGAAGGGG |
| | | AACTGGCAAC ATAGGAGAGA CGCTTGGAGA GAAATGGAAA AGCCGATTGA |
| | | ACGCACTGGG AAAAAGTGAA TTCCAGATCT ACAAGAAAAG TGGAATCCAG |
| | | GAAGTGGATA GAACCTTAGC AAAAGAAGGC ATCAAAAGAG GAGAAACGGA |
| | | CCATCACGCT GTGTCGCGAG GTTCAGCAAA ACTGAGATGG TTCGTTGAGA |
| | | GAAACATGGT CACACCAGAA GGGAAAGTAG TGGACCTCGG TTGTGGCAGA |
| | | GGAGGCTGGT CATACTATTG TGGAGGACTA AAAAACGTAA GAGAAGTCAA |
| | | AGGCCTAACA AAAGGAGGAC CAGGACACGA AGAACCCATC CCCATGTCAA |
| | | CATATGGGTG GAATCTAGTG CGTCTTCAAA GTGGAGTCGA CGTTTTCTTC |
| | | ATCCCGCCAG AAAAGTGTGA CACATTGTTG TGTGACATAG GGGAGTCATC |
| | | ACCAAATCCC ACAGTGGAAG CAGGACGAAC ACTCAGAGTC CTTAATTTAG |
| | | TAGAAAATTG GTTGAACAAC AACACTCAAT TTTGCATAAA AGTTCTCAAC |
| | | CCATATATGC CCTCAGTCAT AGAAAAAATG GAAACACTGC AAAGGAAATA |
| | | TGGAGGAGCC TTAGTGAGGA ATCCACTCTC ACGAAACTCC ACACATGAGA |
| | | TGTACTGGGT ATCTAATGCT TCCGGGAACA TAGTGTCATC AGTGAACATG |
| | | ATTTCAAGGA TGTTGATCAA CAGATTCACA ATGAGACACA AGAAAGCCAC |
| | | TTACGAGCCG GATGTTGACC TCGGAAGCGG AACCCGTAAC ATCGGGATTG |
| | | AAAGTGAGAT ACCAAACCTA GATATAATTG GGAAAAGAAT AGAAAAAATA |
| | | AAGCAAGAGC ATGAAACATC ATGGCACTAT GACCAAGACC ACCCATACAA |
| | | AACGTGGGCA TACCATGGTA GCTATGAAAC AAAACAGACT GGATCAGCAT |
| | | CATCCATGGT CAACGGAGTG GTCAGGCTGC TGACAAAACC TTGGGACGTC |
| | | GTCCCCATGG TGACACAGAT GGCAATGACA GACACGACTC CATTTGGACA |
| | | ACAGCGTGTT TTTAAAGAAA AAGTGGACAC GAGAACCCAA GAACCGAAAG |
| | | AAGGCACGAA GAAACTAATG AAAATCACAG CAGAGTGGCT GTGGAAAGAA |
| | | TTAGGGAAGA AAAAGACACC TAGGATGTGC ACCAGAGAAG AATTCACAAG |
| | | AAAGGTGAGA AGCAATGCAG CCTTGGGGGC CATATTCACT GATGAGAACA |
| | | AGTGGAAGTC GGCACGTGAG GCTGTTGAAG ACAGTAGGTT TTGGGAGCTG |
| | | GTTGACAAGG AAAGGAATCT CCATCTTGAA GGAAAGTGTG AAACATGTGT |
| | | GTACAACATG ATGGGAAAAA GAGAGAAGAA GCTAGGGGAA TTCGGCAAGG |
| | | CAAAAGGTAG CAGAGCCATA TGGTACATGT GGCTTGGAGC TCGCTTCCTA |
| | | GAGTTTGAAG CCCTAGGATT CCTAAATGAA GATCACTGGT TCTCCAGAGA |
| | | GAACTCCCTG AGTGGAGTGG AAGGAGAAGG GCTGCACAAG CTAGGTTACA |
| | | TTCTAAGAGA CGTGAGCAAG AAAGAGGGAG GAGCTATGTA TGCCGATGAC |
| | | ACAGCAGGAT GGGACACAAG AATCACACTA GAAGACCTAA AAAATGAAGA |
| | | AATGGTAACA AACCACATGG AAGGAGAACA CAAGAAACTA GCCGAGGCCA |
| | | TTTTCAAACT AACGTACCAA AACAAGGTGG TGCGTGTGCA AAGACCAACA |
| | | CCAAGAGGCA CAGTAATGGA CATTATATCG AGAAGAGACC AAAGAGGTAG |
| | | TGGACAAGTT GGTACCTATG GACTCAATAC TTTCACCAAT ATGGAAGCCC |
| | | AACTAATCAG ACAGATGGAG GGAGAAGGAG TCTTCAAAAA CATTCAGCAC |
| | | CTGACAGTCA CAGAAGAAAT CGCCGTGCAA AACTGGTTGG CAAGAGTGGG |
| | | GCGCGAAAGG TTATCAAGAA TGGCCATCAG TGGAGATGAT TGTGTTGTGA |
| | | AACCTTTAGA TGACAGGTTC GCAAGCGCTT TAACAGCTCT AAATGACATG |
| | | GGAAAGATTA GGAAAGACAT ACAACAATGG GAACCTTCAA GAGGATGGAA |
| | | CGATTGGACA CAAGTGCCCT TCTGTTCACA CCATTTCCAT GAGCTAATCA |
| | | TGAAAGATGG TCGCGTACTT GTTGTTCCAT GTAGAAACCA AGATGAACTG |
| | | ATTGGCAGAG CCCGAATCTC CCAAGGAGCA GGGTGGTCTT TGCGGGAGAC |
| | | GGCCTGTTTG GGGAAGTCCT ACGCCCAAAT GTGGAGCTTG ATGTACTTCC |
| | | ACAGACGCGA CCTCAGGCTG GCGGCAAATG CCATTTGCTC GGCAGTACCA |
| | | TCACATTGGG TTCCAACAAG TCGAACAACC TGGTCCATAC ACGCCAAACA |
| | | TGAATGGATG ACAACGGAAG ACATGCTGAC AGTCTGGAAC AGGGTGTGGA |
| | | TTCAAGAAAA CCCATGGATG GAAGACAAAA CCCCAGTGGA ATCATGGGAG |
| | | GAAATCCCAT ACTTGGGGAA AAGAGAAGAC CAATGGTGCG GCTCATTGAT |
| | | TGGGTTAACA AGCAGGGCCA CCTGGGCAAA GAACATCCAA GCAGCAATAA |
| | | ATCAAGTTAG ATCCCTTATA GGCAATGAGG AATACACAGA TTACATGCCA |
| | | TCCATGAAAA GATTTAGAAG AGAAGAGGAA GAAGCAGGAG TCCTGTGGTA |
| | | G |
| 3 | DV-3 | GACATGGGAT GTGTTATAAA CTGGAAAGGC AAAGAACTCA AATGTGGAAG |
| | NS1-NS5 | TGGAATCTTC GTCACCAATG AGGTCCATAC CTGGACAGAG CAATACAAAT |
| | NA | TCCAAGCAGA CTCACCAAAA AGATTGGCGA CAGCCATTGC AGGCGCTTGG |
| | | GAGAATGGAG TGTGCGGAAT TAGGTCAACA ACCAGAATGG AGAATCTCCT |
| | | GTGGAAGCAA ATAGCCAATG AACTGAACTA CATATTGTGG GAAAACAATA |
| | | TCAAATTAAC GGTAGTTGTG GGCGATATAA TTGGGGTCTT AGAGCAAGGA |
| | | AAAAGAACAC TAACACCACA ACCCATGGAG CTAAAATACT CATGGAAAAC |
| | | GTGGGGAAAG GCAAAAATAG TGACAGCTGA AATACAAAAT TCCTCTTTCA |
| | | TAATAGACGG GCCAAACACA CCGGAGTGTC CAAGTGCCTC AAGAGCATGG |
| | | AATGTGTGGG AGGTGGAAGA TTACGGGTTC GGAGTCTTCA CAACCAACAT |
| | | ATGGCTGAAA CTCCGAGAGG TGTACACCCA ACTATGTGAC CATAGGCTAA |
| | | TGTCGGCAGC TGTCAAGGAT GAGAGGGCCG TGCATGCCGA CATGGGCTAC |
| | | TGGATAGAAA GCCAAAAGAA TGGAAGTTGG AAGCTAGAAA AAGCATCCCT |
| | | CATAGAGGTG AAAACCTGCA CATGGCCAAA ATCACATACC CTTTGGAGTA |
| | | ATGGTGTGCT AGAGAGTGAC ATGATCATCC CAAAGAGTCT AGCTGGTCCT |
| | | ATTTCGCAAC ACAACTACAG GCCCGGGTAC CACACCCAGA CGGCGGGACC |
| | | CTGGCATTTA GGAAAATTAG ACCTGGACTT CAACTATTGC GAAGGAACAA |
| | | CAGTTGTCAT CACAGAAAAC TGTGGGACAA GAGGCCCATC ATTGAGAACA |
| | | ACAACAGTGT CAGGGAAGTT AATACACGAA TGGTGTTGCC GTTCGTGCAC |
| | | ACTTCCTCCC TTGCGATACA TGGGAGAAGA CGGTTGCTGG TATGGCATGG |
| | | AAATCAGACC CATCAGTGAG AAAGAAGAGA ACATGGTAAA GTCTTTAGTC |
| | | TCAGCGGGAA GTGGAAAGGT GGACAACTTT ACAATGGGTG TCTTGTGTTT |
| | | GGCAATCCTC TTTGAAGAGG TGATGAGAGG AAAATTTGGG AAGAAACACA |
| | | TGATTGCGGG GGTTTTCTTC ACGTTTGTGC TCCTTCTCTC AGGGCAAATA |
| | | ACATGGAGAG ACATGGCGCA CACACTAATA ATGATTGGGT CCAACGCATC |
| | | TGACAAGATG GGAATGGGCG TTACCTACCT AGCTTTAATT GCAACATTTA |
| | | AAATCCAGCC ATTCTTGGCT TTGGGATTTT TCCTAAGAAA ACTGACATCC |
| | | AGAGAAAATT TATTGTTAGG AGTTGGGCTG GCTATGGCAA CAACGTTACA |
| | | ACTGCCAGAG GACATTGAAC AAATGGCAAA TGGAATCGCT CTGGGGCTCA |
| | | TGGCGCTTAA ACTGATAACA CAATTTGAAA CATACCAATT ATGGACAGCA |
| | | TTAATCTCCT TAACGTGTTC AAATACAATG TTTACGTTGA CTGTTGCCTG |
| | | GAGAACAGCC ACCCTGATTT TGGCCGGAGT TTCGCTTTTA CCAGTGTGTC |
| | | AGTCTTCGAG CATGAGGAAA ACAGACTGGC TTCCAATGGC AGTGGCAGCT |
| | | ATGGGAGTTC CACCTCTACC ACTTTTTATT TTTAGCTTGA AAGACACACT |
| | | TAAAAGGAGA AGCTGGCCAC TGAATGAAGG GGTGATGGCT GTTGGGCTTG |
| | | TGAGCATTCT GGCCAGTTCT CTCCTTAGAA ATGATGTACC CATGGCTGGA |
| | | CCATTAGTGG CCGGGGGCTT GCTGATAGCG TGCTACGTCA TAACTGGCAC |
| | | GTCAGCAGAC CTCACCGTAG AAAAAGCAGC AGATATAACA TGGGAGGAAG |
| | | AGGCTGAGCA AACAGGAGTG TCCCACAACT TAATGATCAC AGTTGATGAT |
| | | GATGGAACAA TGAGAATAAA AGATGATGAG ACTGAGAATA TCCTAACAGT |
| | | GCTTTTGAAA ACAGCATTAC TAATAGTATC AGGAGTCTTT CCATACTCCA |
| | | TACCCGCAAC ATTGCTGGTC TGGCACACTT GGCAAAAGCA AACCCAAAGA |
| | | TCCGGCGTTC TATGGGACGT ACCCAGCCCC CCAGAGACAC AGAAAGCAGA |
| | | ACTGGAAGAG GGGGTCTATA GGATCAAACA GCAAGGAATT TTTGGGAAAA |
| | | CCCAAGTAGG GGTTGGAGTA CAGAAAGAAG GAGTCTTCCA CACCATGTGG |
| | | CACGTTACAA GAGGGGCGGT GTTGACATAT AATGGGAAAA GACTGGAACC |
| | | AAACTGGGCT AGTGTGAAAA AAGATCTGAT TTCATACGGA GGAGGATGGA |
| | | GATTGAGCGC ACAATGGCAA AAGGGGGAGG AGGTGCAGGT TATTGCCGTA |
| | | GAGCCTGGGA AGAACCCAAA GAACTTTCAA ACCATGCCAG GCACTTTTCA |
| | | GACTACAACA GGGGAAATAG GAGCAATTGC ACTGGATTTC AAGCCTGGAA |
| | | CTTCAGGATC TCCTATCATA AACAGAGAGG GAAAGGTAGT GGGACTGTAT |
| | | GGCAATGGAG TGGTTACAAA GAATGGTGGC TACGTCAGCG GAATAGCGCA |
| | | AACGAATGCA GAACCAGATG GACCGACACC AGAATTGGAA GAAGAGATGT |
| | | TCAAAAAGCG AAATCTAACC ATAATGGATC TTCATCCTGG GTCAGGAAAG |
| | | ACACGGAAAT ACCTTCCAGC TATTGTTAGA GAGGCAATCA AGAGACGTCT |
| | | AAGGACTCTA ATTTTGGCAC CGACAAGGGT GGTTGCAGCT GAGATGGAAG |
| | | AAGCATTGAA AGGGCTCCCA ATAAGGTACC AAACAACAGC AACAAAATCT |
| | | GAACACACAG GAAGAGAGAT TGTTGATCTA ATGTGCCACG CAACGTTCAC |
| | | AATGCGCCTG CTGTCACCAG TTAGGGTTCC AAATTATAAC TTGATAATAA |
| | | TGGATGAAGC CCATTTCACA GACCCAGCCA GTATAGCGGC TAGAGGGTAC |
| | | ATATCGACTC GTGTTGGAAT GGGAGAGGCA GCCGCAATTT TCATGACAGC |
| | | AACGCCCCCT GGAACAGCTG ATGCCTTTCC TCAGAGCAAC GCTCCAATTC |
| | | AAGATGAAGA AAGGGACATA CCAGAACGCT CATGGAATTC AGGCAATGAA |
| | | TGGATAACCG ACTTCGCTGG GAAAACGGTG TGGTTTGTCC CCAGCATTAA |
| | | AGCCGGAAAT GACATAGCAA ACTGCTTGCG AAAAAACGGG AAAAAGGTCA |
| | | TTCAACTTAG TAGGAAGACT TTTGACACAG AATATCAGAA AACTAAACTG |
| | | AATGATTGGG ACTTCGTAGT GACAACTGAC ATTTCAGAAA TGGGGGCCAA |
| | | TTTCAAAGCA GATAGAGTGA TCGACCCAAG AAGATGTCTC AAACCAGTGA |
| | | TCCTGACAGA TGGACCAGAG CGGGTGATCC TGGCTGGACC AATGCCAGTC |
| | | ACCGCGGCGA GTGCTGCGCA AAGGAGAGGG AGAGTTGGCA GGAACCCACA |
| | | AAAAGAAAAT GACCAGTACA TATTCACGGG CCAACCTCTC AATAATGATG |
| | | AAGACCACGC TCACTGGACA GAAGCAAAAA TGCTGCTGGA CAACATTAAT |
| | | ACACCAGAAG GGATCATACC AGCTCTCTTT GAGCCAGAAA GGGAGAAGTC |
| | | AGCCGCCATA GACGGTGAGT ATCGCTTGAA AGGTGAGTCT AGGAAGACTT |
| | | TCGTGGAACT CATGAGGAGG GGTGACCTTC CAGTCTGGTT AGCCCATAAA |
| | | GTAGCATCAG AAGGGATCAA ATATACAGAT AGAAAATGGT GCTTTGATGG |
| | | ACAACGTAAT AATCAAATTT TAGAGGAGAA CATGGATGTG GAAATCTGGA |
| | | CAAAGGAAGG AGAAAAGAAA AAATTGAGAC CTAGGTGGCT TGATGCCCGC |
| | | ACTTATTCAG ATCCCTTAGC ACTCAAGGAA TTCAAGGACT TTGCGGCTGG |
| | | CAGAAAGTCA ATCGCCCTTG ATCTTGTGAC AGAAATAGGA AGAGTGCCTT |
| | | CACATCTAGC CCATAGAACG AGAAACGCTC TGGACAATCT GGTGATGCTG |
| | | CATACGTCAG AACATGGCGG TAGGGCCTAC AGGCATGCGG TGGAGGAACT |
| | | ACCAGAGACA ATGGAAACAC TCCTACTCTT GGGACTCATG ATCTTGTTGA |
| | | CAGGTGGAGC AATGCTTTTC TTAATATCAG GTAAAGGGAT TGGAAAGACC |
| | | TCAATAGGAC TCATTTGTGT AATTGCTTCC AGCGGCATGT TGTGGATGGC |
| | | CGAAATCCCA CTCCAATGGA TCGCGTCGGC TATAGTCCTG GAGTTTTTTA |
| | | TGATGGTGTT GCTTATACCA GAACCAGAAA AGCAGAGAAC TCCCCAAGAC |
| | | AACCAACTCG CATATGTCGT GATAGGCATA CTCACATTGG CTGCAATAAT |
| | | AGCAGCCAAT GAAATGGGAT TGTTGGAAAC TACAAAGAGA GATTTAGGAA |
| | | TGTCTAAGGA GCCAGGTGTT GTCTCTCCAA CCAGCTATTT AGATGTGGAC |
| | | TTGCACCCAG CATCAGCCTG GACATTGTAC GCCGTGGCCA CTACAGTAAT |
| | | AACACCAATG TTAAGACATA CCATAGAGAA TTCTACAGCA AATGTGTCTC |
| | | TGGCAGCTAT AGCCAACCAG GCAGTGGTCC TGATGGGTTT GGACAAAGGA |
| | | TGGCCAATAT CAAAAATGGA CTTAGGAGTA CCCCTACTGG CATTGGGTTG |
| | | CTATTCACAA GTGAACCCAC TGACTCTAAC AGCGGCAGTA CTCTTGCTGA |
| | | TCACACATTA TGCTATTATA GGTCCAGGAT TGCAGGCAAA AGCCACTCGT |
| | | GAAGCTCAAA AAAGGACAGC TGCTGGAATA ATGAAGAATC CAACGGTGGA |
| | | TGGGATAATG ACAATAGACC TAGATCCCGT AATATATGAT TCAAAATTTG |
| | | AAAAACAACT GGGACAGGTT ATGCTCCTGG TTTTGTGTGC AGTTCAACTT |
| | | TTGTTAATGA GAACATCATG GGCCTTGTGT GAAGCTTTAA CTCTAGCTAC |
| | | AGGACCAATA ACAACACTCT GGGAAGGATC ACCTGGGAAG TTTTGGAACA |
| | | CCACGATAGC TGTTTCCATG GCGAACATTT TTAGAGGGAG CTATTTAGCA |
| | | GGAGCTGGGC TTGCTTTTTC TATAATGAAA TCAGTTGGAA CAGGAAAAAG |
| | | AGGAACAGGC TCACAAGGTG AAACTTTAGG AGAAAAATGG AAAAAGAAAT |
| | | TAAATCAATT ATCCCGGAAA GAGTTTGACC TTTACAAGAA ATCTGGAATC |
| | | ACTGAAGTGG ATAGAACAGA AGCCAAAGAA GGGTTGAAAA GAGGAGAAAC |
| | | AACACATCAT GCCGTGTCCA GAGGTACCGC GAAACTTCAA TGGTTTGTGG |
| | | AGAGAAACAT GGTCATTCCC GAAGGAAGAG TCATAGACTT GGGCTGTGGA |
| | | AGAGGAGGCT GGTCATATTA CTGTGCAGGA CTGAAAAAAG TCACAGAAGT |
| | | GCGAGGATAC ACAAAAGGCG GTCCAGGACG CGAAGAACCA GTACCTATGT |
| | | CCACATATGG ATGGAACATA GTTAAGTTAA TGAGTGGAAA GGATGTGTTT |
| | | TATCTTCCAC CTGAAAAGTG TGACACCCTG TTGTGTGACA TTGGAGAATC |
| | | TTCACCAAGC CCAACAGTGG AAGAAAGCAG AACTATAAGA GTTTTGAAGA |
| | | TGGTTGAACC ATGGCTAAAG AACAACCAAT TTTGCATTAA AGTATTGAAC |
| | | CCTTACATGC CAACTGTGAT TGAGCACCTA GAAAGATTAC AAAGGAAACA |
| | | TGGAGGAATG CTTGTGAGAA ATCCACTTTC ACGAAACTCC ACGCACGAAA |
| | | TGTACTGGAT ATCTAATGGC ACAGGTAACA TTGTCGCTTC AGTCAATATG |
| | | GTATCTAGAC TGCTACTGAA CAGGTTCACG ATGACACACA GAAGACCCAC |
| | | CATTGAGAAA GATGTGGATT TAGGAGCAGG AACTCGACAT GTTAATGCGG |
| | | AACCAGAAAC ACCCAACATG GATGTCATTG GGGAAAGAAT AAAAAGGATC |
| | | AAGGAAGAGC ATAATTCAAC ATGGCACTAT GATGACGAAA ACCCCTACAA |
| | | AACGTGGGCT TACCATGGAT CTTATGAAGT TAAAGCCACA GGCTCAGCCT |
| | | CCTCCATGAT AAATGGAGTC GTGAAACTCC TCACTAAACC ATGGGATGTG |
| | | GTGCCCATGG TGACACAGAT GGCAATGACA GATACAACTC CATTTGGCCA |
| | | GCAGAGAGTC TTTAAAGAGA AAGTGGACAC CAGGACACCC AGGTCCATGC |
| | | CAGGAACAAG AAGGGTCATG GGGATCACAG CGGAGTGGCT CTGGAGAACC |
| | | TTGGGAAGGA ACAAAAAACC CAGGCTATGC ACAAGGGAAG AGTTTACAAA |
| | | AAAGGTCAGA ACCAACGCAG CCATGGGCGC CGTTTTCACA GAGGAGAACC |
| | | AATGGGATAG CGCGAAAGCT GCTGTTGAGG ATGAGGATTT TTGGAAACTT |
| | | GTGGACAGAG AACGTGAACT CCACAAATTG GGCAAGTGTG GAAGCTGTGT |
| | | TTACAACATG ATGGGCAAGA GAGAGAAGAA ACTTGGAGAG TTTGGCAAAG |
| | | CAAAAGGCAG TAGAGCTATA TGGTACATGT GGTTGGGAGC CAGGTACCTT |
| | | GAGTTCGAAG CCCTTGGATT CTTAAATGAA GACCACTGGT TCTCGCGTGA |
| | | GAACTCTTAC AGTGGAGTGG AAGGAGAAGG ACTGCACAAG CTAGGCTATA |
| | | TATTAAGGGA CATTTCCAAG ATACCCGGAG GAGCTATGTA TGCTGATGAC |
| | | ACAGCTGGTT GGGACACAAG AATAACAGAA GATGACCTGC ACAATGAGGA |
| | | AAAGATCACA CAGCAAATGG ACCCTGAACA CAGGCAGTTA GCGAACGCTA |
| | | TATTTAAGCT CACATACCAA AACAAAGTGG TCAAAGTTCA ACGACCGACT |
| | | CCAACAGGCA CGGTAATGGA CATCATATCT AGGAAAGACC AAAGAGGCAG |
| | | TGGACAGGTA GGAACTTATG GTCTGAATAC ATTCACCAAC ATGGAAGCCC |
| | | AGTTAATCAG ACAAATGGAA GGAGAAGGTG TGCTGTCAAA GGCAGACCTC |
| | | GAGAACCCTC ATCTGCCAGA GAAGAAAATT ACACAATGGT TGGAAACCAA |
| | | AGGAGTGGAG AGGTTAAGAA GAATGGCCAT TAGCGGGGAT GATTGCGTAG |
| | | TGAAACCAAT CGATGACAGG TTCGCTAATG CCCTGCTTGC TCTGAACGAC |
| | | ATGGGAAAGG TTCGGAAAGA CATACCTCAA TGGCAGCCAT CAAAGGGATG |
| | | GCATGATTGG CAACAGGTTC CTTTCTGCTC CCACCACTTT TCATGAATTG |
| | | ATCATGAAAG ATGGAAGAAA GTTGGTGGTT CCCTGCAGAC CCCAGGACGA |
| | | ACTAATAGGA AGAGCAAGAA TCTCTCAAGG AGCGGGATGG AGCCTTAGAG |
| | | AAACCGCATG TCTGGGGAAA GCCTACGCTC AAATGTGGAG TCTCATGTAT |
| | | TTTCACAGAA GAGACCTCAG ACTAGCATCC AACGCCATAT GTTCAGCAGT |
| | | ACCAGTCCAC TGGGTCCCCA CAAGTAGAAC GACATGGTCT ATTCATGCTC |
| | | ACCATCAGTG GATGACCACA GAAGACATGC TTACTGTCTG GAACAGGGTA |
| | | TGGATCGAGG ACAATCCATG GATGGAAGAC AAAACTCCAG TCACAACCTG |
| | | GGAAAATGTT CCATATTTAG GGAAGAGAGA AGACCAATGG TGCGGATCAC |
| | | TTATTGGTCT CACTTCCAGA GCAACCTGGG CCCAGAACAT ACCCACAGCA |
| | | ATTCAACAGG TGAGAAGCCT TATAGGCAAT GAAGAGTTTC TGGACTACAT |
| | | GCCTTCAATG AAGAGATTTA GGAAGGAGGA GGAGTCGGAG GGAGCCATTT |
| | | GGTAA |
| 4 | DV-4 | GACATGGGTT GTGTGGCGTC ATGGAGTGGG AAAGAATTGA AGTGTGGAAG |
| | NS1-NS5 | CGGAATTTTT GTGGTTGACA ACGTGCACAC TTGGACAGAA CAGTACAAAT |
| | NA | TTCAACCAGA GTCCCCAGCG AGACTAGCGT CTGCAATATT AAATGCCCAC |
| | | AAAGATGGGG TCTGTGGAAT TAGATCAACC ACGAGGCTGG AAAATGTCAT |
| | | GTGGAAGCAA ATAACCAACG AGCTAAACTA TGTTCTCTGG GAAGGAGGAC |
| | | ATGACCTCAC TGTAGTGGCT GGGGATGTGA AGGGGGTGTT GACCAAAGGC |
| | | AAGAGAGCAC TCACACCCCC AGTGAGTGAT CTGAAATATT CATGGAAGAC |
| | | ATGGGGAAAA GCAAAAATCT TCACCCCAGA AGCAAGAAAT AGCACATTTT |
| | | TAATAGACGG ACCAGACACC TCTGAATGCC CCAATGAACG AAGAGCATGG |
| | | AACTCTCTTG AGGTGGAAGA CTATGGATTT GGCATGTTCA CGACCAACAT |
| | | ATGGATGAAA TTCCGAGAAG GAAGTTCAGA AGTGTGTGAC CACAGGTTAA |
| | | TGTCAGCTGC AATTAAAGAT CAGAAAGCTG TGCATGCTGA CATGGGTTAT |
| | | TGGATAGAGA GCTCAAAAAA CCAGACCTGG CAGATAGAGA AAGCATCTCT |
| | | TATTGAAGTG AAAACATGTC TGTGGCCCAA GACCCACACA CTGTGGAGCA |
| | | ATGGAGTGCT GGAAAGCCAG ATGCTCATTC CAAAATCATA TGCGGGCCCT |
| | | TTTTCACAGC ACAATTACCG CCAGGGCTAT GCCACGCAAA CCGTGGGCCC |
| | | ATGGCACTTA GGCAAATTAG AGATAGACTT TGGAGAATGC CCCGGAACAA |
| | | CAGTCACAAT TCAGGAGGAT TGTGACCATA GAGGCCCATC TTTGAGGACC |
| | | ACCACTGCAT CTGGAAAACT AGTCACGCAA TGGTGCTGCC GCTCCTGCAC |
| | | GATGCCTCCC TTAAGGTTCT TGGGAGAAGA TGGGTGCTGG TATGGGATGG |
| | | AGATTAGGCC CTTGAGTGAA AAAGAAGAGA ACATGGTCAA ATCACAGGTG |
| | | ACGGCCGGAC AGGGCACATC AGAAACTTTT TCTATGGGTC TGTTGTGCCT |
| | | GACCTTGTTT GTGGAAGAAT GCTTGAGGAG AAGAGTCACT AGGAAACACA |
| | | TGATATTAGT TGTGGTGATC ACTCTTTGTG CTATCATCCT GGGAGGCCTC |
| | | ACATGGATGG ACTTACTACG AGCCCTCATC ATGTTGGGGG ACACTATGTC |
| | | TGGTAGAATA GGAGGACAGA TCCACCTAGC CATCATGGCA GTGTTCAAGA |
| | | TGTCACCAGG ATACGTGCTG GGTGTGTTTT TAAGGAAACT CACTTCAAGA |
| | | GAGACAGCAC TAATGGTAAT AGGAATGGCC ATGACAACGG TGCTTTCAAT |
| | | TCCACATGAC CTTATGGAAC TCATTGATGG AATATCACTG GGACTAATTT |
| | | TGCTAAAAAT AGTAACACAG TTTGACAACA CCCAAGTGGG AACCTTAGCT |
| | | CTTTCCTTGA CTTTCATAAG ATCAACAATG CCATTGGTCA TGGCTTGGAG |
| | | GACCATTATG GCTGTGTTGT TTGTGGTCAC ACTCATTCCT TTGTGCAGGA |
| | | CAAGCTGTCT TCAAAAACAG TCTCATTGGG TAGAAATAAC AGCACTCATC |
| | | CTAGGAGCCC AAGCTCTGCC AGTGTACCTA ATGACTCTTA TGAAAGGAGC |
| | | CTCAAGAAGA TCTTGGCCTC TTAACGAGGG CATAATGGCT GTGGGTTTGG |
| | | TTAGTCTCTT AGGAAGCGCT CTTTTAAAGA ATGATGTCCC TTTAGCTGGC |
| | | CCAATGGTGG CAGGAGGCTT ACTTCTGGCG GCTTACGTGA TGAGTGGTAG |
| | | CTCAGCAGAT CTGTCACTAG AGAAGGCCGC CAACGTGCAG TGGGATGAAA |
| | | TGGCAGACAT AACAGGCTCA AGCCCAATCG TAGAAGTGAA GCAGGATGAA |
| | | GATGGCTCTT TCTCCATACG GGACGTCGAG GAAACCAATA TGATAACCCT |
| | | TTTGGTGAAA CTGGCACTGA TAACAGTGTC AGGTCTCTAC CCCTTGGCAA |
| | | TTCCAGTCAC AATGACCTTA TGGTACATGT GGCAAGTGAA AACACAAAGA |
| | | TCAGGAGCCC TGTGGGACGT CCCCTCACCC GCTGCCACTA AAAAAGCCGC |
| | | ACTGTCTGAA GGAGTGTACA GGATCATGCA AAGAGGGTTA TTCGGGAAAA |
| | | CTCAGGTTGG AGTAGGGATA CACATGGAAG GTGTATTTCA CACAATGTGG |
| | | CATGTAACAA GAGGATCAGT GATCTGCCAC GAGACTGGGA GATTGGAGCC |
| | | ATCTTGGGCT GACGTCAGGA ATGACATGAT ATCATACGGT GGGGGATGGA |
| | | GGCTTGGAGA CAAATGGGAC AAAGAAGAAG ACGTTCAGGT CCTCGCCATA |
| | | GAACCAGGAA AAAATCCTAA ACATGTCCAA ACGAAACCTG GCCTTTTCAA |
| | | GACCCTAACT GGAGAAATTG GAGCAGTAAC ATTAGATTTC AAACCCGGAA |
| | | CGTCTGGTTC TCCCATCATC AACAGGAAAG GAAAAGTCAT CGGACTCTAT |
| | | GGAAATGGAG TAGTTACCAA ATCAGGTGAT TACGTCAGTG CCATAACGCA |
| | | AGCCGAAAGA ATTGGAGAGC CAGATTATGA AGTGGATGAG GACATTTTTC |
| | | GAAAGAAAAG ATTAACTATA ATGGACTTAC ACCCCGGAGC TGGAAAGACA |
| | | AAAAGAATTC TTCCATCAAT AGTGAGAGAA GCCTTAAAAA GGAGGCTACG |
| | | AACTTTGATT TTAGCTCCCA CGAGAGTGGT GGCGGCCGAG ATGGAAGAGG |
| | | CCCTACGTGG ACTGCCAATC CGTTATCAGA CCCCAGCTGT GAAATCAGAA |
| | | CACACAGGAA GAGAGATTGT AGACCTCATG TGTCATGCAA CCTTCACAAC |
| | | AAGACTTTTG TCATCAACCA GGGTTCCAAA TTACAACCTT ATAGTGATGG |
| | | ATGAAGCACA TTTCACCGAT CCTTCTAGTG TCGCGGCTAG AGGATACATC |
| | | TCGACCAGGG TGGAAATGGG AGAGGCAGCA GCCATCTTCA TGACCGCAAC |
| | | CCCTCCCGGA GCGACAGATC CCTTTCCCCA GAGCAACAGC CCAATAGAAG |
| | | ACATCGAGAG GGAAATTCCG GAAAGGTCAT GGAACACAGG GTTCGACTGG |
| | | ATAACAGACT ACCAAGGGAA AACTGTGTGG TTTGTTCCCA GCATAAAAGC |
| | | TGGAAATGAC ATTGCAAATT GTTTGAGAAA GTCGGGAAAG AAAGTTATCC |
| | | AGTTGAGTAG GAAAACCTTT GATACAGAGT ATCCAAAAAC GAAACTCACG |
| | | GACTGGGACT TTGTGGTCAC TACAGACATA TCTGAAATGG GGGCCAATTT |
| | | TAGAGCCGGG AGAGTGATAG ACCCTAGAAG ATGCCTCAAG CCAGTTATCC |
| | | TACCAGATGG GCCAGAGAGA GTCATTTTAG CAGGTCCTAT TCCAGTGACT |
| | | CCAGCAAGCG CTGCTCAGAG AAGAGGGCGA ATAGGAAGGA ACCCAGCACA |
| | | AGAAGACGAC CAATACGTTT TCTCCGGAGA CCCACTAAAA AATGATGAAG |
| | | ATCATGCCCA CTGGACAGAA GCAAAGATGC TGCTTGACAA TATCTACACC |
| | | CCAGAAGGGA TCATTCCAAC ATTGTTTGGT CCGGAAAGGG AAAAAACCCA |
| | | AGCCATTGAT GGAGAGTTTC GCCTCAGAGG GGAACAAAGG AAGACTTTTG |
| | | TGGAATTAAT GAGGAGAGGA GACCTTCCGG TGTGGCTGAG CTATAAGGTA |
| | | GCTTCTGCTG GCATTTCTTA CGAAGATCGG GAATGGTGCT TCACAGGGGA |
| | | AAGAAATAAC CAAATTTTAG AAGAAAACAT GGAGGTTGAA ATTTGGACTA |
| | | GAGAGGGAGA AAAGAAAAAG CTAAGGCCAA GATGGTTAGA TGCACGTGTA |
| | | TACGCTGACC CCATGGCTTT GAAGGATTTC AAGGAGTTTG CCAGTGGAAG |
| | | GAAGAGTATA ACTCTCGACA TCCTAACAGA GATTGCCAGT TTGCCAACTT |
| | | ACCTTTCCTC TAGGGCCAAG CTCGCCCTTG ATAACATAGT CATGCTCCAC |
| | | ACAACAGAAA GAGGAGGGAG GGCCTATCAA CACGCCCTGA ACGAACTTCC |
| | | GGAGTCACTG GAAACACTCA TGCTTGTAGC TTTACTAGGT GCTATGACAG |
| | | CAGGCATCTT CCTGTTTTTC ATGCAAGGGA AAGGAATAGG GAAATTGTCA |
| | | ATGGGTTTGA TAACCATTGC GGTGGCTAGT GGCTTGCTCT GGGTAGCAGA |
| | | AATTCAACCC CAGTGGATAG CGGCCTCAAT CATACTAGAG TTTTTTCTCA |
| | | TGGTACTGTT GATACCGGAA CCAGAAAAAC AAAGGACCCC ACAAGACAAT |
| | | CAATTGATCT ACGTCATATT GACCATTCTC ACCATCATTG GTCTAATAGC |
| | | AGCCAACGAG ATGGGGCTGA TTGAAAAAAC AAAAACGGAT TTTGGGTTTT |
| | | ACCAGGTAAA AACAGAAACC ACCATCCTCG ATGTGGACTT GAGACCAGCT |
| | | TCAGCATGGA CGCTCTATGC AGTAGCCACC ACAATTCTGA CTCCCATGCT |
| | | GAGACACACC ATAGAAAACA CGTCGGCCAA CCTATCTCTA GCAGCCATTG |
| | | CCAACCAGGC AGCCGTCCTA ATGGGGCTTG GAAAAGGATG GCCGCTCCAC |
| | | AGAATGGACC TCGGTGTGCC GCTGTTAGCA ATGGGATGCT ATTCTCAAGT |
| | | GAACCCAACA ACCTTGACAG CATCCTTAGT CATGCTTTTA GTCCATTATG |
| | | CAATAATAGG CCCAGGATTG CAGGCAAAAG CCACAAGAGA GGCCCAGAAA |
| | | AGGACAGCTG CTGGGATCAT GAAAAATCCC ACAGTGGACG GGATAACAGT |
| | | AATAGATCTA GAACCAATAT CCTATGACCC AAAATTTGAA AAGCAATTAG |
| | | GGCAGGTCAT GCTACTAGTC TTGTGTGCTG GACAACTACT CTTGATGAGA |
| | | ACAACATGGG CTTTCTGTGA AGTCTTGACT TTGGCCACAG GACCAATCTT |
| | | GACCTTGTGG GAGGGCAACC CGGGAAGGTT TTGGAACACG ACCATAGCCG |
| | | TATCCACCGC CAACATTTTC AGGGGAAGTT ACTTGGCGGG AGCTGGACTG |
| | | GCTTTTTCAC TCATAAAGAA TGCACAAACC CCTAGGAGGG GAACTGGGAC |
| | | CACAGGAGAG ACACTGGGAG AGAAGTGGAA GAGACAGCTA AACTCATTAG |
| | | ACAGAAAAGA GTTTGAAGAG TATAAAAGAA GTGGAATACT AGAAGTGGAC |
| | | AGGACTGAAG CCAAGTCTGC CCTGAAAGAT GGGTCTAAAA TCAAGCATGC |
| | | AGTATCAAGA GGGTCCAGTA AGATCAGATG GATTGTTGAG AGAGGGATGG |
| | | TAAAGCCAAA AGGGAAAGTT GTAGATCTTG GCTGTGGGAG AGGAGGATGG |
| | | TCTTATTACA TGGCGACACT CAAGAACGTG ACTGAAGTGA AAGGGTATAC |
| | | AAAAGGAGGT CCAGGACATG AAGAACCGAT TCCCATGGCT ACTTATGGTT |
| | | GGAATTTGGT CAAACTCCAT TCAGGGGTTG ACGTGTTCTA CAAACCCACA |
| | | GAGCAAGTGG ACACCCTGCT CTGTGATATT GGGGAGTCAT CTTCTAATCC |
| | | AACAATAGAG GAAGGAAGAA CATTAAGAGT TTTGAAGATG GTGGAGCCAT |
| | | GGCTCTCTTC AAAACCTGAA TTCTGCATCA AAGTCCTTAA CCCCTACATG |
| | | CCAACAGTCA TAGAAGAGCT GGAGAAACTG CAGAGAAAAC ATGGTGGGAA |
| | | CCTTGTCAGA TGCCCGCTGT CCAGGAACTC CACCCATGAG ATGTATTGGG |
| | | TGTCAGGAGC GTCGGGAAAC ATTGTGAGCT CTGTGAACAC AACATCAAAG |
| | | ATGTTGTTGA ACAGGTTCAC AACAAGGCAT AGGAAACCCA CTTATGAGAA |
| | | GGACGTAGAT CTTGGGGCAG GAACGAGAAG TGTCTCCACT GAAACAGAAA |
| | | AACCAGACAT GACAATCATT GGGAGAAGGC TTCAGCGATT GCAAGAAGAG |
| | | CACAAAGAAA CCTGGCATTA TGATCAGGAA AACCCATACA GAACCTGGGC |
| | | GTATCATGGA AGCTATGAAG CTCCTTCGAC AGGCTCTGCA TCCTCCATGG |
| | | TGAACGGGGT GGTAAAACTG CTAACAAAAC CCTGGGATGT GATTCCAATG |
| | | GTGACTCAGT TAGCCATGAC AGATACAACC CCTTTTGGGC AACAAAGAGT |
| | | GTTCAAAGAG AAGGTGGATA CCAGAACACC ACAACCAAAA CCCGGTACAC |
| | | GAATGGTTAT GACCACGACA GCCAATTGGC TGTGGGCCCT CCTTGGAAAG |
| | | AAGAAAAATC CCAGACTGTG CACAAGGGAA GAGTTCATCT CAAAAGTTAG |
| | | ATCAAACGCA GCCATAGGCG CAGTCTTTCA GGAAGAACAG GGATGGACAT |
| | | CAGCCAGTGA AGCTGTGAAT GACAGCCGGT TTTGGGAACT GGTTGACAAA |
| | | GAAAGGGCCC TACACCAGGA AGGGAAATGT GAATCGTGTG TCTATAACAT |
| | | GATGGGAAAA CGTGAGAAAA AGTTAGGAGA GTTTGGCAGA GCCAAGGGAA |
| | | GCCGAGCAAT CTGGTACATG TGGCTGGGAG CGCGGTTTCT GGAATTTGAA |
| | | GCCCTGGGTT TTTTGAATGA AGATCACTGG TTTGGCAGAG AAAATTCATG |
| | | GAGTGGAGTG GAAGGGGAAG GTCTGCACAG ATTGGGATAT ATCCTGGAGG |
| | | AGATAGACAA GAAGGATGGA GACCTAATGT ATGCTGATGA CACAGCAGGC |
| | | TGGGACACAA GAATCACTGA GGATGACCTT CAAAATGAGG AACTGATCAC |
| | | GGAACAGATG GCTCCCCACC ACAAGATCCT AGCCAAAGCC ATTTTCAAAC |
| | | TAACCTATCA AAACAAAGTG GTGAAAGTCC TCAGACCCAC ACCGCGGGGA |
| | | GCGGTGATGG ATATCATATC CAGGAAAGAC CAAAGAGGTA GTGGACAAGT |
| | | TGGAACATAT GGTTTGAACA CATTCACCAA CATGGAAGTT CAACTCATCC |
| | | GCCAAATGGA AGCTGAAGGA GTCATCACAC AAGATGACAT GCAGAACCCA |
| | | AAAGGGTTGA AAGAAAGAGT TGAGAAATGG CTGAAAGAGT GTGGTGTCGA |
| | | CAGGTTAAAG AGGATGGCAA TCAGTGGAGA CGATTGCGTG GTGAAGCCCC |
| | | TAGATGAGAG GTTTGGCACT TCCCTCCTCT TCTTGAACGA CATGGGAAAG |
| | | GTGAGGAAAG ACATTCCGCA GTGGGAACCA TCTAAGGGAT GGAAAAACTG |
| | | GCAAGAGGTT CCTTTTTGCT CCCACCACTT TCACAAGATC TTTATGAAGG |
| | | ATGGCCGCTC ACTAGTTGTT CCATGTAGAA ACCAGGATGA ACTGATAGGG |
| | | AGAGCCAGAA TCTCGCAGGG AGCTGGATGG AGCTTAAGAG AAACAGCCTG |
| | | CCTGGGCAAA GCTTACGCCC AGATGTGGTC GCTTATGTAC TTCCACAGAA |
| | | GGGATCTGCG TTTAGCCTCC ATGGCCATAT GCTCAGCAGT TCCAACGGAA |
| | | TGGTTTCCAA CAAGCAGAAC AACATGGTCA ATCCACGCTC ATCACCAGTG |
| | | GATGACCACT GAAGATATGC TCAAAGTGTG GAACAGAGTG TGGATAGAAG |
| | | ACAACCCTAA TATGACTGAC AAGACTCCAG TCCATTCGTG GGAAGATATA |
| | | CCTTACCTAG GGAAAAGAGA GGATTTGTGG TGTGGATCCC TGATTGGACT |
| | | TTCTTCCAGA GCCACCTGGG CGAAGAACAT TCATACGGCC ATAACCCAGG |
| | | TCAGGAACCT GATCGGAAAA GAGGAATACG TGGATTACAT GCCAGTAATG |
| | | AAAAGATACA GTGCTCCTTC AGAGAGTGAA GGAGTTCTGT AA |
| 5 | DV-1 | AGTTGTTAGT CTACGTGGAC CGACAAGAAC AGTTTCGAAT CGGAAGCTTG |
| | 5′UTR | CTTAACGTAG TTCTAACAGT TTTTTATTAG AGAGCAGCAT |
| 6 | DV-2 | AGTTGTTAGT CTACGTGGAC CGACAAAGAC AGATTCTTTG AGGGAGCTAA |
| | 5′UTR | GCTCAACGTA GTTCTAACAG TTTTTTAATT AGAGAGCAGA TCTCTG |
| 7 | DV-3 | GACTCGGAAG CTTGCTTAAC GTAGTGCTAA CAGTTTTTTG TTAGAGAGCA |
| | 5′UTR | GATCTCTG |
| 8 | DV-4 | AGTTGTTAGT CTGTGTGGAC CGACAAGGAC AGTTCCAAAT CGGAAGCTTG |
| | 5′UTR | CTTAACACAG TTCTAACAGT TTGTTTGAAT AGAGAGCAGA TCTCTGGAAA |
| | | A |
| 9 | DV-1 | GCCAACTCAT TCACAAAATA AAGGAAAATA AAAAATCAAA CAAGGCAAGA |
| | 3′UTR | AGTCAGGCCG GATTAAGCCA TAGCACGGTA AGAGCTATGC TGCCTGTGAG |
| | | CCCCGTCCAA GGACGTAAAA TGAAGTCAGG CCGAAAGCCA CGGTTCGAGC |
| | | AAGCCGTGCT GCCTGTAGCT CCATCGTGGG GATGTAAAAA CCCGGGAGGC |
| | | TGCAAACCAT GGAAGCTGTA CGCATGGGGT AGCAGACTAG TGGTTAGAGG |
| | | AGACCCCTCC CAAGACACAA CGCAGCAGCG GGGCCCAACA CCAGGGGAAG |
| | | CTGTACCCTG GTGGTAAGGA CTAGAGGTTA GAGGAGACCC CCCGCACAAC |
| | | ATATTGACGC AACAAACAGC TGGGAGAGAC CAGAGATCCT GCTGTCTCTA |
| | | CAGCATCATT CCAGGCACAG AACGCCAAAA AATGGAATGG TGCTGTTGAA |
| | | TCAACAGGTT CT |
| 10 | DV-2 | AAAGCAGAAC TAATTTAAAA CAAGGCTGGA AGTCAGGTCG GATTAAGCCA |
| | 3′UTR | TAGTACGGAA AAAACTATGC TACCTGTGAG CCCCGTCCAA GGACGTTAAA |
| | | AGAAGTCAGG CCATCATAAA TGCCATAGCT TGAGTAAACT ATGCAGCCTG |
| | | TAGCTCCACC TGAGAAGGTG TAAAAAATCC GGGAGGCCAC AAACCATGGA |
| | | AGCTGTACGC ATGGCGTAGT GGACTAGCGG TTAGAGGAGA CCCCTCCCTT |
| | | ACAAATCGCA GCAACAATGG GGGCCCAAGG CGAGATGAAG CTGTAGTCTC |
| | | GCTGGAAGGA CTAGAGGTTA GAGGAGACCC CCCCGAAACA AAAAACAGCA |
| | | TATTGACGCT GGGAAAGACC AGAGATCCTG CTGTCTCCTC AGCATCATTC |
| | | CAGGCACAGA ACGCCAGAAA ATGGAATGGT GCTGTTGAAT CAACAGGTTC |
| | | T |
| 11 | DV-3 | ACGTAGGAAG TGAAAAAGAG GCTAACTGTC AGGCCACATT AAGCCACAGT |
| | 3′UTR | ACGGAAGAAG CTGTGCTGCC TGTGAGCCCC GTCCAAGGAC GTTAAAAGAA |
| | | GAAGTCAGGC CCCAAAGCCA CGGTTTGAGC AAACCGTGCT GCCTGTAGCT |
| | | CCGTCGTGGG GACGTAAAAC CTGGGAGGCT GCAAACTGTG GAAGCTGTAC |
| | | GCACGGTGTA GCAGACTAGC GGTTAGAGGA GACCCCTCCC ATGACACAAC |
| | | GCAGCAGCGG GGCCCGAGCA CTGAGGGAAG CTGTACCTCC TTGCAAAGGA |
| | | CTAGAGGTTA GAGGAGACCC CCCGCAAATA AAAACAGCAT ATTGACGCTG |
| | | GGAGAGACCA GAGATCCTGC TGTCTCCTCA GCATCATTCC AGGCACAGAA |
| | | CGCCAGAAAA TGGAATGGTG CTGTTGAATC AACAGGTTCT |
| 12 | DV-4 | TTACCAACAA CAAACACCAA AGGCTATTGA AGTCAGGCCA CTTGTGCCAC |
| | 3′UTR | GGTTTGAGCA AACCGTGCTG CCTGTAGCTC CGCCAATAAT GGGAGGCGTA |
| | | ATAATCCCCA GGGAGGCCAT GCGCCACGGA AGCTGTACGC GTGGCATATT |
| | | GGACTAGCGG TTAGAGGAGA CCCCTCCCAT CACTGATAAA ACGCAGCAAA |
| | | AGGGGGCCCG AAGCCAGGAG GAAGCTGTAC TCCTGGTGGA AGGACTAGAG |
| | | GTTAGAGGAG ACCCCCCCAA CACAAAAACA GCATATTGAC GCTGGGAAAG |
| | | ACCAGAGATC CTGCTGTCTC TGCAACATCA ATCCAGGCAC AGAGCGCCGC |
| | | AAGATGGATT GGTGTTGTTG ATCCAACAGG TTCT |
| 13 | DV-1 | ATGAACAACC AACGGAAAAA GACGGGTCGA CCGTCTTTCA ATATGCTGAA |
| | partial | ACGCGCGAGA AACCGCGTGT CAACTGTTTC ACAGTTGGCG AAGAGATTCT |
| | CNA | CA |
| 14 | DV-2 | ATGAATAACC AACGGAAAAA GGCGAGAAAC ACGCCTTTCA ATATGCTGAA |
| | partial | ACGCGAGAGA AACCGCGTGT CGACTGTGCA ACAGCTGACA AAGAGATTCT |
| | CNA | CA |
| 15 | DV-3 | ATGAACAACC AACGGAAGAA GGCGGGAAAA CCGTCTATCA ATATGCTGAA |
| | partial | ACGCGTGAGA AACCGTGTGT CAACTGGATC ACAGTTGGCG AAGAGATTCT |
| | CNA | CA |
| 16 | DV-4 | ATGAACCAAC GAAAAAAGGT GGTTAGACCA CCTTTCAATA TGCTGAAACG |
| | partial | CGAGAGAAAC CGCGTATCAA CCCCTCAAGG GTTGGTGAAG AGATTCTCA |
| | CNA | |
| 17 | DV-1 | ATTCTGCTGA CATGGCTAGG ATTAAACTCA AGGAGCACGT CCCTTTCAAT |
| | partial E | GACGTGTATC GCAGTTGGCA TGGTCACACT GTACCTAGGA GTCATGGTTC |
| | ENA | AGGCG |
| 18 | DV-2 | GTCATTATCA CATGGATAGG AATGAATTCA CGCAGCACCT CACTGTCTGT |
| | partial E | GTCACTAGTA TTGGTGGGAA TTGTGACACT GTATTTGGGA GTCATGGTGC |
| | ENA | AGGCG |
| 19 | DV-3 | GTTCTCTTGA CTTGGATAGG GTTGAATTCA AAAAACACAT CCATGTCATT |
| | partial E | TTCATGCATT GCGATAGGAG TCATTACACT CTATCTGGGA GCTGTGGTGC |
| | ENA | AAGCT |
| 20 | DV-4 | TTCTTAGTGT TGTGGATTGG CACGAACTCG AGGAACACTT CAATGGCTAT |
| | partial E | GACGTGCATA GCTGTTGGAG GAATCACTCT GTTTCTGGGC TTCACAGTTC |
| | ENA | AAGCA |
| 21 | HDV | GGGTCGGCAT GGCATCTCCA CCTCCTCGCG GTCCGACCTG GGCATCCGAA |
| | ribozyme | GGAGGACGCA CGTCCACTCG GATGGCTAAG |
| 22 | DV-1 | DSGCVINWKG RELKCGSGIF VTNEVHTWTE QYKFQADSPK RLSAAIGKAW |
| | NS1-NS5 | EEGVCGIRSA TRLENIMWKQ ISNELNHILL ENDMKFTVVV GDVSGILAQG |
| | AA | KKMIRPQPME HKYSWKSWGK AKIIGADVQN TTFIIDGPNT PECPDNQRAW |
| | | NIWEVEDYGF GIFTTNIWLK LRDSYTQVCD HRLMSAAIKD SKAVHADMGY |
| | | WIESEKNETW KLARASFIEV KTCIWPKSHT LWSNGVLESE MIIPKIYGGP |
| | | ISQHNYRPGY FTQTAGPWHL GKLELDEDLC EGTTVVVDEH CGNRGPSLRT |
| | | TTVTGKTIHE WCCRSCTLPP LRFKGEDGCW YGMEIRPVKE KEENLVKSMV |
| | | SAGSGEVDSF SLGLLCISIM IEEVMRSRWS RKMLMTGTLA VFLLLTMGQL |
| | | TWNDLIRLCI MVGANASDKM GMGTTYLALM ATERMRPMFA VGLLERRLTS |
| | | REVLLLTVGL SLVASVELPN SLEELGDGLA MGIMMLKLLT DFQSHQLWAT |
| | | LLSLTFVKTT FSLHYAWKTM AMILSIVSLF PLCLSTTSQK TTWLPVLLGS |
| | | LGCKPLTMEL ITENKIWGRK SWPLNEGIMA VGIVSILLSS LLKNDVPLAG |
| | | PLIAGGMLIA CYVISGSSAD LSLEKAAEVS WEEEAEHSGA SHNILVEVQD |
| | | DGTMKIKDEE RDDTLTILLK ATLLAISGVY PMSIPATLFV WYFWQKKKQR |
| | | SGVLWDTPSP PEVERAVLDD GIYRILQRGL GGWRFQGSWN FQEGVFHTMW |
| | | HVTRGAVLMY QGKRLEPSWA SVKKDLISYG LGRSQVGVGV AGEEVQVIAV |
| | | EPGKNPKNVQ TAPGTFKTPE GEVGAIALDF KPGTSGSPIV NREGKIVGLY |
| | | GNGVVTTSGT YVSAIAQAKA SQEGPLPEIE DEVERKRNLT IMDLHPGSGK |
| | | TRRYLPAIVR EAIKRKLRTL VLAPTRVVAS EMAEALKGMP IRYQTTAVKS |
| | | EHTGKEIVDL MCHATFTMRL LSPVRVPNYN MIIMDEAHFT DPASIAARGY |
| | | ISTRVGMGEA AAIFMTATPP GSVEAFPQSN AVIQDEERDI PERSWNSGYD |
| | | WITDFPGKTV WFVPSIKSGN DIANCLRKNG KRVVQLSRKT FDTEYQKTKN |
| | | NDWDYVVTTD ISEMGANFRA DRVIDPRRCL KPVILKDGPE RVILAGPMPV |
| | | TVASAAQRRG RIGRNQNKEG DQYIYMGQPL NNDEDHAHWT EAKMLLDNIN |
| | | TPEGIIPALF EPEREKSAAI DGEYRLRGEA RKTFVELMRR GDLPVWLSYK |
| | | VASEGFQYSD RRWCFDGERN NQVLEENMDV EIWTKEGERK KLRPRWLDAR |
| | | TYSDPLALRE FKEFAAGRRS VSGDLILEIG KLPQHLTQRA QNALDNLVML |
| | | HNSEQGGKAY RHAMEELPDT IETLMLLALI AVLTGGVTLF FLSGRGLGKT |
| | | SIGLLCVIAS SALLWMASVE PHWIAASIIL EFFLMVLLIP EPDRQRTPQD |
| | | NQLAYVVIGL LFMILTVAAN EMGLLETTKK DLGIGHAAAE NHHHAAMLDV |
| | | DLHPASAWTL YAVATTIITP MMRHTIENTT ANISLTAIAN QAAILMGLDK |
| | | GWPISKMDIG VPLLALGCYS QVNPLTLTAA VLMLVAHYAI IGPGLQAKAT |
| | | REAQKRTAAG IMKNPTVDGI VAIDLDPVVY DAKFEKQLGQ IMLLILCTSQ |
| | | ILLMRTTWAL KSLGGGRRGT GAQGETLGEK WKRQLNQLSK SEENTYKRSG |
| | | AGAGLAFSLM EGLKRGETTK HAVSRGTAKL RWFVERNLVK PEGKVIDLGC |
| | | IIEVDRSEAK CESITLATGP LTTLWEGSPG KFWNTTIAVS MANIERGSYL |
| | | GRGGWSYYCA GLKKVTEVKG YTKGGPGHEE PIPMATYGWN LVKLYSGKDV |
| | | FFTPPEKCDT LLCDIGESSP NPTIEEGRTL RVLKMVEPWL RGNQFCIKIL |
| | | NPYMPSVVET LEQMQRKHGG MLVRNPLSRN STHEMYWVSC GTGNIVSAVN |
| | | MTSRMLLNRF TMAHRKPTYE RDVDLGAGTR HVAVEPEVAN LDIIGQRIEN |
| | | IKNEHKSTWH YDEDNPYKTW AYHGSYEVKP SGSASSMVNG VVRLLTKPWD |
| | | VIPMVTQIAM TDTTPFGQQR VEKEKVDTRT PKAKRGTAQI MEVTARWLWG |
| | | FLSRNKKPRI CTREEFTRKV RSNAAIGAVE VDENQWNSAK EAVEDEREWD |
| | | LVHRERELHK QGKCATCVYN MMGKREKKLG EFGKAKGSRA IWYMWLGARE |
| | | LEFEALGEMN EDHWESRENS LSGVEGEGLH KLGYILRDIS KIPGGNMYAD |
| | | DTAGWDTRIT EDDLQNEAKI TDIMEPEHAL LATSIFKLTY QNKVVRVQRP |
| | | AKNGTVMDVI SRRDQRGSGQ VGTYGLNTFT NMEAQLIRQM ESEGIFSPSE |
| | | LETPNLAERV LDWLKKHGTE RLKRMAISGD DCVVKPIDDR FATALTALND |
| | | MGKVRKDIPQ WEPSKGWNDW QQVPFCSHHF HQLIMKDGRE IVVPCRNQDE |
| | | LVGRARVSQG AGWSLRETAC LGKSYAQMWQ LMYFHRRDLR LAANAICSAV |
| | | PVDWVPTSRT TWSIHAHHQW MTTEDMLSVW NRVWIEENPW MEDKTHVSSW |
| | | EDVPYLGKRE DQWCGSLIGL TARATWATNI QVAINQVRRL IGNENYLDEM |
| | | TSMKRFKNES DPEGALW- |
| 23 | DV-2 | DSGCVVSWKN KELKCGSGIF ITDNVHTWTE QYKFQPESPS KLASAIQKAQ |
| | NS1-NS5 | EEGICGIRSV TRLENLMWKQ ITPELNHILA ENEVKLTIMT GDIKGIMQAG |
| | AA | KRSLRPQPTE LKYSWKTWGK AKMLSTESHN QTFLIDGPET AECPNTNRAW |
| | | NSLEVEDYGF GVFTTNIWLK LKEKQDAFCD SKLMSAAIKD NRAVHADMGY |
| | | WIESALNDTW KIEKASFIEV KNCHWPKSHT LWSNGVLESE MIIPKNLAGP |
| | | VSQHNYRPGY HTQIAGPWHL GKLEMDEDFC DGTTVVVTED CGNRGPSLRT |
| | | TTASGKLITE WCCRSCTLPP LRYRGEDGCW YGMEIRPLKE KEENLVNSLV |
| | | TAGHGQVDNF SLGVLGMALF LEEMLRTRVG TKHAILLVAV SFVTLITGNM |
| | | SFRDLGRVVV MVGATMTDDI GMGVTYLALL AAFKVRPTFA AGLLLRKLTS |
| | | KELMMTTIGI VLLSQSTIPE TILELTDALA LGMMVLKMVR NMEKYQLAVT |
| | | IMAILCVPNA VILQNAWKVS CTILAVVSVS PLLLTSSQQK TDWIPLALTI |
| | | KGLNPTAIFL TTLSRTSKKR SWPLNEAIMA VGMVSILASS LLKNDIPMTG |
| | | PLVAGGLLTV CYVLTGRSAD LELERAADVK WEDQAEISGS SPILSITISE |
| | | DGSMSIKNEE EEQTLTILIR TGLLVISGLF PVSIPITAAA WYLWEVKKQR |
| | | AGVLWDVPSP PPMGKAELED GAYRIKQKGI LGYSQIGAGV YKEGTFHTMW |
| | | HVTRGAVLMH KGKRIEPSWA DVKKDLISYG GGWKLEGEWK EGEEVQVLAL |
| | | EPGKNPRAVQ TKPGLERTNA GTIGAVSLDE SPGTSGSPII DKKGKVVGLY |
| | | GNGVVTRSGA YVSAIAQTEK SIEDNPEIED DIFRKRRLTI MDLHPGAGKT |
| | | KRYLPAIVRE AIKRGLRTLI LAPTRVVAAE MEEALRGLPI RYQTPAIRAE |
| | | HTGREIVDLM CHATFTMRLL SPVRVPNYNL IIMDEAHFTD PASIAARGYI |
| | | STRVEMGEAA GIFMTATPPG SRDPFPQSNA PIIDEEREIP ERSWNSGHEW |
| | | VTDFKGKTVW FVPSIKAGND IAACLRKNGK KVIQLSRKTF DSEYVKTRTN |
| | | DWDFVVTTDI SEMGANFKAE RVIDPRRCMK PVILTDGEER VILAGPMPVT |
| | | HSSAAQRRGR IGRNPKNEND QYIYMGEPLE NDEDCAHWKE AKMLLDNINT |
| | | PEGIIPSMFE PEREKVDAID GEYRLRGEAR KTFVDLMRRG DLPVWLAYKV |
| | | AAEGINYADR RWCEDGIKNN QILEENVEVE IWTKEGERKK LKPRWLDARI |
| | | YSDPLALKEF KEFAAGRKSL TLNLITEMGR LPTEMTQKAR DALDNLAVLH |
| | | TAEAGGRAYN HALSELPETL ETLLLLTLLA TVTGGIFLFL MSGRGIGKMT |
| | | LGMCCIITAS ILLWYAQIQP HWIAASIILE FFLIVLLIPE PEKQRTPQDN |
| | | QLTYVVIAIL TVVAATMANE MGFLEKTKKD LGLGSIATQQ PESNILDIDL |
| | | RPASAWTLYA VATTFVTPML RHSIENSSVN VSLTAIANQA TVLMGLGKGW |
| | | PLSKMDIGVP LLAIGCYSQV NPITLTAALL LLVAHYAIIG PGLQAKATRE |
| | | AQKRAAAGIM KNPTVDGITV IDLDPIPYDP KFEKQLGQVM LLVLCVTQVL |
| | | MMRTTWALCE ALTLATGPIS TLWEGNPGRE WNTTIAVSMA NIFRGSYLAG |
| | | AGLLFSIMKN TTNTRRGTGN IGETLGEKWK SRLNALGKSE FQIYKKSGIQ |
| | | EVDRTLAKEG IKRGETDHHA VSRGSAKLRW FVERNMVTPE GKVVDLGCGR |
| | | GGWSYYCGGL KNVREVKGLT KGGPGHEEPI PMSTYGWNLV RLQSGVDVFF |
| | | IPPEKCDTLL CDIGESSPNP TVEAGRTLRV LNLVENWLNN NTQFCIKVLN |
| | | PYMPSVIEKM ETLQRKYGGA LVRNPLSRNS THEMYWVSNA SGNIVSSVNM |
| | | ISRMLINRFT MRHKKATYEP DVDLGSGTRN IGIESEIPNL DIIGKRIEKI |
| | | KQEHETSWHY DQDHPYKTWA YHGSYETKQT GSASSMVNGV VRLLTKPWDV |
| | | VPMVTQMAMT DTTPFGQQRV FKEKVDTRTQ EPKEGTKKLM KITAEWLWKE |
| | | LGKKKTPRMC TREEFTRKVR SNAALGAIFT DENKWKSARE AVEDSREWEL |
| | | VDKERNLHLE GKCETCVYNM MGKREKKLGE FGKAKGSRAI WYMWLGAREL |
| | | EFEALGELNE DHWFSRENSL SGVEGEGLHK LGYILRDVSK KEGGAMYADD |
| | | TAGWDTRITL EDLKNEEMVT NHMEGEHKKL AEAIFKLTYQ NKVVRVQRPT |
| | | PRGTVMDIIS RRDQRGSGQV GTYGLNTFTN MEAQLIRQME GEGVEKNIQH |
| | | LTVTEEIAVQ NWLARVGRER LSRMAISGDD CVVKPLDDRF ASALTALNDM |
| | | GKIRKDIQQW EPSRGWNDWT QVPFCSHHFH ELIMKDGRVL VVPCRNQDEL |
| | | IGRARISQGA GWSLRETACL GKSYAQMWSL MYFHRRDLRL AANAICSAVP |
| | | SHWVPTSRTT WSIHAKHEWM TTEDMLSVWN RVWIQENPWM EDKTPVESWE |
| | | EIPYLGKRED QWCGSLIGLT SRATWAKNIQ AAINQVRSLI GNEEYTDYMP |
| | | SMKRFRREEE EAGVLW- |
| 24 | DV-3 | DMGCVINWKG KELKCGSGIF VTNEVHTWTE QYKFQADSPK RLATAIAGAW |
| | NS1-NS5 | ENGVCGIRST TRMENLLWKQ IANELNYILW ENNIKLTVVV GDIIGVLEQG |
| | AA | KRTLTPQPME LKYSWKTWGK AKIVTAEIQN SSFIIDGPNT PECPSASRAW |
| | | NVWEVEDYGF GVFTTNIWLK LREVYTQLCD HRLMSAAVKD ERAVHADMGY |
| | | WIESQKNGSW KLEKASLIEV KTCTWPKSHT LWSNGVLESD MIIPKSLAGP |
| | | ISQHNYRPGY HTQTAGPWHL GKLDLDENYC EGTTVVITEN CGTRGPSLRT |
| | | TTVSGKLIHE WCCRSCTLPP LRYMGEDGCW YGMEIRPISE KEENMVKSLV |
| | | SAGSGKVDNF TMGVLCLAIL FEEVMRGKFG KKHMIAGVFF TFVLLLSGQI |
| | | TWRDMAHTLI MIGSNASDKM GMGVTYLALI ATFKIQPFLA LGFFLRKLTS |
| | | RENLLLGVGL AMATTLQLPE DIEQMANGIA LGLMALKLIT QFETYQLWTA |
| | | LISLTCSNTM FTLTVAWRTA TLILAGVSLL PVCQSSSMRK TDWLPMAVAA |
| | | MGVPPLPLFI FSLKDTLKRR SWPLNEGVMA VGLVSILASS LLRNDVPMAG |
| | | PLVAGGLLIA CYVITGTSAD LTVEKAADIT WEEEAEQTGV SHNLMITVDD |
| | | DGTMRIKDDE TENILTVLLK TALLIVSGVF PYSIPATLLV WHTWQKQTQR |
| | | SGVLWDVPSP PETQKAELEE GVYRIKQQGI FGKTQVGVGV QKEGVFHTMW |
| | | HVTRGAVLTY NGKRLEPNWA SVKKDLISYG GGWRLSAQWQ KGEEVQVIAV |
| | | EPGKNPKNFQ TMPGTFQTTT GEIGAIALDF KPGTSGSPII NREGKVVGLY |
| | | GNGVVTKNGG YVSGIAQTNA EPDGPTPELE EEMEKKRNLT IMDLHPGSGK |
| | | TRKYLPAIVR EAIKRRLRTL ILAPTRVVAA EMEEALKGLP IRQTTATKSE |
| | | HTGREIVDLM CHATFTMRLL SPVRVPNYNL IIMDEAHFTD PASIAARGYI |
| | | STRVGMGEAA IFMTATPPGT ADAFPQSNAP IQDEERDIPE RSWNSGNEWI |
| | | TDFAGKTVWF PSIKAGNDIA NCLRKNGKKV IQLSRKTEDT EYQKTKLNDW |
| | | DFVVTTDISE MGANFKADRV IDPRRCLKPV ILTDGPERVI LAGPMPVTAA |
| | | SAAQRRGRVG RNPQKENDQY IFTGQPLNND EDHAHWTEAK MLLDNINTPE |
| | | GIIPALFEPE REKSAAIDGE YRLKGESRKT FVELMRRGDL PVWLAHKVAS |
| | | EGIKYTDRKW CFDGQRNNQI LEENMDVEIW TKEGEKKKLR PRWLDARTYS |
| | | DPLALKEFKD FAAGRKSIAL DLVTEIGRVP SHLAHRTRNA LDNLVMLHTS |
| | | EHGGRAYRHA VEELPETMET LLLLGLMILL TGGAMLFLIS GKGIGKTSIG |
| | | LICVIASSGM LWMAEIPLQW IASAIVLEFF MMVLLIPEPE KQRTPQDNQL |
| | | AYVVIGILTL AAIIAANEMG LLETTKRDLG MSKEPGVVSP TSYLDVDLHP |
| | | ASAWTLYAVA TTVITPMLRH TIENSTANVS LAAIANQAVV LMGLDKGWPI |
| | | SKMDLGVPLL ALGCYSQVNP LTLTAAVLLL ITHYAIIGPG LQAKATREAQ |
| | | KRTAAGIMKN PTVDGIMTID LDPVIYDSKF EKQLGQVMLL VLCAVQLLLM |
| | | RTSWALCEAL TLATGPITTL WEGSPGKEWN TTIAVSMANI FRGSYLAGAG |
| | | LAFSIMKSVG TGKRGTGSQG ETLGEKWKKK LNQLSRKEFD LYKKSGITEV |
| | | DRTEAKEGLK RGETTHHAVS RGTAKLQWFV ERNMVIPEGR VIDLGCGRGG |
| | | WSYYCAGLKK VTEVRGYTKG GPGHEEPVPM STYGWNIVKL MSGKDVFYLP |
| | | PEKCDTLLCD IGESSPSPTV EESRTIRVLK MVEPWLKNNQ FCIKVLNPYM |
| | | PTVIEHLERL QRKHGGMLVR NPLSRNSTHE MYWISNGTGN IVASVNMVSR |
| | | LLLNRFTMTH RRPTIEKDVD LGAGTRHVNA EPETPNMDVI GERIKRIKEE |
| | | HNSTWHYDDE NPYKTWAYHG SYEVKATGSA SSMINGVVKL LTKPWDVVPM |
| | | VTQMAMTDTT PFGQQRVEKE KVDTRTPRSM PGTRRVMGIT AEWLWRTLGR |
| | | NKKPRLCTRE EFTKKVRTNA AMGAVETEEN QWDSAKAAVE DEDFWKLVDR |
| | | ERELHKLGKC GSCVYNMMGK REKKLGEFGK AKGSRAIWYM WLGARYLEFE |
| | | ALGELNEDHW FSRENSYSGV EGEGLHKLGY ILRDISKIPG GAMYADDTAG |
| | | WDTRITEDDL HNEEKITQQM DPEHRQLANA IFKLTYQNKV VKVQRPTPTG |
| | | TVMDIISRKD QRGSGQVGTY GLNTFTNMEA QLIRQMEGEG VLSKADLENP |
| | | HLPEKKITQW LETKGVERLR RMAISGDDCV VKPIDDRFAN ALLALNDMGK |
| | | VRKDIPQWQP SKGWHDWQQV PFCSHHFHEL IMKDGRKLVV PCRPQDELIG |
| | | RARISQGAGW SLRETACLGK AYAQMWSLMY FHRRDLRLAS NAICSAVPVH |
| | | WVPTSRTTWS IHAHHQWMTT EDMLTVWNRV WIEDNPWMED KTPVTTWENV |
| | | PYLGKREDQW CGSLIGLTSR ATWAQNIPTA IQQVRSLIGN EEFLDYMPSM |
| | | KRERKEEESE GAIW- |
| 25 | DV-4 | DMGCVASWSG KELKCGSGIF VVDNVHTWTE QYKFQPESPA RLASAILNAH |
| | NS1-NS5 | KDGVCGIRST TRLENVMWKQ ITNELNYVLW EGGHDLTVVA GDVKGVLTKG |
| | AA | KRALTPPVSD LKYSWKTWGK AKIFTPEARN STELIDGPDT SECPNERRAW |
| | | NSLEVEDYGF GMFTTNIWMK FREGSSEVCD HRLMSAAIKD QKAVHADMGY |
| | | WIESSKNQTW QIEKASLIEV KTCLWPKTHT LWSNGVLESQ MLIPKSYAGP |
| | | FSQHNYRQGY ATQTVGPWHL GKLEIDFGEC PGTTVTIQED CDHRGPSLRT |
| | | TTASGKLVTQ WCCRSCTMPP LRELGEDGCW YGMEIRPLSE KEENMVKSQV |
| | | TAGQGTSETF SMGLLCLTLF VEECLRRRVT RKHMILVVVI TLCAIILGGL |
| | | TWMDLLRALI MLGDTMSGRI GGQIHLAIMA VFKMSPGYVL GVFLRKLTSR |
| | | ETALMVIGMA MTTVLSIPHD LMELIDGISL GLILLKIVTQ FDNTQVGTLA |
| | | LSLTFIRSTM PLVMAWRTIM AVLFVVTLIP LCRTSCLQKQ SHWVEITALI |
| | | LGAQALPVYL MTLMKGASRR SWPLNEGIMA VGLVSLLGSA LLKNDVPLAG |
| | | PMVAGGLLLA AYVMSGSSAD LSLEKAANVQ WDEMADITGS SPIVEVKQDE |
| | | DGSFSIRDVE ETNMITLLVK LALITVSGLY PLAIPVTMTL WYMWQVKTQR |
| | | SGALWDVPSP AATKKAALSE GVYRIMQRGL FGKTQVGVGI HMEGVFHTMW |
| | | HVTRGSVICH ETGRLEPSWA DVRNDMISYG GGWRLGDKWD KEEDVQVLAI |
| | | EPGKNPKHVQ TKPGLFKTLT GEIGAVTLDF KPGTSGSPII NRKGKVIGLY |
| | | GNGVVTKSGD YVSAITQAER IGEPDYEVDE DIFRKKRLTI MDLHPGAGKT |
| | | KRILPSIVRE ALKRRLRTLI LAPTRVVAAE MEEALRGLPI RYQTPAVKSE |
| | | HTGREIVDLM CHATFTTRLL SSTRVPNYNL IVMDEAHFTD PSSVAARGYI |
| | | STRVEMGEAA IFMTATPPGA TDPFPQSNSP IEDIEREIPE RSWNTGEDWI |
| | | TDYQGKTVWF VPSIKAGNDI ANCLRKSGKK VIQLSRKTED TEYPKTKLTD |
| | | WDFVVTTDIS EMGANFRAGR VIDPRRCLKP VILPDGPERV ILAGPIPVTP |
| | | ASAAQRRGRI GRNPAQEDDQ YVESGDPLKN DEDHAHWTEA KMLLDNIYTP |
| | | EGIIPTLFGP EREKTQAIDG EFRLRGEQRK TEVELMRRGD LPVWLSYKVA |
| | | SAGISYEDRE WCFTGERNNQ ILEENMEVEI WTREGEKKKL RPRWLDARVY |
| | | ADPMALKDEK EFASGRKSIT LDILTEIASL PTYLSSRAKL ALDNIVMLHT |
| | | TERGGRAYQH ALNELPESLE TLMLVALLGA MTAGIFLFFM QGKGIGKLSM |
| | | GLITIAVASG LLWVAEIQPQ WIAASIILEF FLMVLLIPEP EKQRTPQDNQ |
| | | LIYVILTILT IIGLIAANEM GLIEKTKTDF GFYQVKTETT ILDVDLRPAS |
| | | AWTLYAVATT ILTPMLRHTI ENTSANLSLA AIANQAAVLM GLGKGWPLHR |
| | | MDLGVPLLAM GCYSQVNPTT LTASLVMLLV HYAIIGPGLQ AKATREAQKR |
| | | TAAGIMKNPT VDGITVIDLE PISYDPKFEK QLGQVMLLVL CAGQLLLMRT |
| | | TWAFCEVLTL ATGPILTLWE GNPGREWNTT IAVSTANIFR GSYLAGAGLA |
| | | FSLIKNAQTP RRGTGTTGET LGEKWKRQLN SLDRKEFEEY KRSGILEVDR |
| | | TEAKSALKDG SKIKHAVSRG SSKIRWIVER GMVKPKGKVV DLGCGRGGWS |
| | | YYMATLKNVT EVKGYTKGGP GHEEPIPMAT YGWNLVKLHS GVDVFYKPTE |
| | | QVDTLLCDIG ESSSNPTIEE GRTLRVLKMV EPWLSSKPEF CIKVLNPYMP |
| | | TVIEELEKLQ RKHGGNLVRC PLSRNSTHEM YWVSGASGNI VSSVNTTSKM |
| | | LLNRFTTRHR KPTYEKDVDL GAGTRSVSTE TEKPDMTIIG RRLQRLQEEH |
| | | KETWHYDQEN PYRTWAYHGS YEAPSTGSAS SMVNGVVKLL TKPWDVIPMV |
| | | TQLAMTDTTP FGQQRVFKEK VDTRTPQPKP GTRMVMTTTA NWLWALLGKK |
| | | KNPRLCTREE FISKVRSNAA IGAVFQEEQG WTSASEAVND SRFWELVDKE |
| | | RALHQEGKCE SCVYNMMGKR EKKLGEFGRA KGSRAIWYMW LGARFLEFEA |
| | | LGFLNEDHWF GRENSWSGVE GEGLHRLGYI LEEIDKKDGD LMYADDTAGW |
| | | DTRITEDDLQ NEELITEQMA PHHKISAKAI FKLTYQNKVV KVLRPTPRGA |
| | | VMDIISRKDQ RGSGQVGTYG LNTFTNMEVQ LIRQMEAEGV ITQDDMQNPK |
| | | GLKERVEKWL KECGVDRLKR MAISGDDCVV KPLDERFGTS LLFLNDMGKV |
| | | RKDIPQWEPS KGWKNWQEVP FCSHHEHKIF MKDGRSLVVP CRNQDELIGR |
| | | ARISQGAGWS LRETACLGKA YAQMWSLMYF HRRDLRLASM AICSAVPTEW |
| | | FPTSRTTWSI HAHHQWMTTE DMLKVWNRVW IEDNPNMTDK TPVHSWEDIP |
| | | YLGKREDLWC GSLIGLSSRA TWAKNIHTAI TQVRNLIGKE EYVDYMPVMK |
| | | RYSAPSESEG VL- |
| 26 | DV-1 | MNNQRKKTGR PSENMLKRAR NRVSTVSQLA KARFS |
| | partial | |
| | AA | |
| 27 | DV-2 | MNNQRKKARN TPENMLKRER NRVSTVQQLT KRES |
| | partial | |
| | AA | |
| 28 | DV-3 | MNNQRKKAGK PSINMLKRVR NRVSTGSQLA KRES |
| | partial | |
| | AA | |
| 29 | DV-4 | MNQRKKVVRP PENMLKRERN RVSTPQGLVK RES |
| | partial | |
| | AA | |
| 30 | DV-1 | ILLTWLGLNS RSTSLSMTCI AVGMVTLYLG VMVQA |
| | partial | |
| | EAA | |
| 31 | DV-2 | VIITWIGMNS RSTSLSVSLV LVGIVTLYLG VMVQA |
| | partial | |
| | EAA | |
| 32 | DV-3 | VLLTWIGLNS KNTSMSFSCI AIGVITLYLG AVVQA |
| | partial | |
| | EAA | |
| 33 | DV-4 | FLVLWIGTNS RNTSMAMTCI AVGGITLFLG FTVQA |
| | partial | |
| | EAA | |
| 34 | ZV CME | ATGAAAAACC CAAAAAAGAA ATCCGGAGGA TTCCGGATTG TCAATATGCT |
| | NA | AAAACGCGGA GTAGCCCGTG TGAGCCCCTT TGGGGGCTTG AAGAGGCTGC |
| | | CAGCCGGACT TCTGCTGGGT CATGGGCCCA TCAGGATGGT CTTGGCGATT |
| | | CTAGCCTTTT TGAGATTCAC GGCAATCAAG CCATCACTGG GTCTCATCAA |
| | | TAGATGGGGT TCAGTGGGGA AAAAAGAGGC TATGGAAACA ATAAAGAAGT |
| | | TCAAGAAAGA TCTGGCTGCC ATGCTGAGAA TAATCAATGC TAGGAAGGAG |
| | | AAGAAGAGAC GAGGCGCAGA TACTAGTGTC GGAATTGTTG GCCTCCTGCT |
| | | GACCACAGCT ATGGCAGCGG AGGTCACTAG ACGTGGGAGT GCATACTATA |
| | | TGTACTTGGA CAGAAACGAT GCTGGGGAGG CCATATCTTT TCCAACCACA |
| | | TTGGGGATGA ATAAGTGTTA TATACAGATC ATGGATCTTG GACACATGTG |
| | | TGATGCCACC ATGAGCTATG AATGCCCTAT GCTGGATGAG GGGGTGGAAC |
| | | CAGATGACGT CGATTGTTGG TGCAACACGA CGTCAACTTG GGTTGTGTAC |
| | | GGAACCTGCC ATCACAAAAA AGGTGAAGCA CGGAGATCTA GAAGAGCTGT |
| | | GACGCTCCCC TCCCATTCCA CCAGGAAGCT GCAAACGCGG TCGCAAACCT |
| | | GGTTGGAATC AAGAGAATAC ACAAAGCACT TGATTAGAGT CGAAAATTGG |
| | | ATATTCAGGA ACCCTGGCTT CGCGTTAGCA GCGGCCGCCA TCGCTTGGCT |
| | | TTTGGGAAGC TCAACGAGCC AAAAAGTCAT ATACTTGGTC ATGATACTGC |
| | | TGATTGCCCC GGCATACAGC ATCAGGTGCA TAGGAGTCAG CAATAGGGAC |
| | | TTTGTGGAAG GTATGTCAGG TGGGACTTGG GTTGATGTTG TCTTGGAACA |
| | | TGGAGGTTGT GTCACCGTAA TGGCACAGGA CAAACCGACT GTCGACATAG |
| | | AGCTGGTTAC AACAACAGTC AGCAACATGG CGGAGGTAAG ATCCTACTGC |
| | | TATGAGGCAT CAATATCAGA CATGGCTTCT GACAGCCGCT GCCCAACACA |
| | | AGGTGAAGCC TACCTTGACA AGCAATCAGA CACTCAATAT GTCTGCAAAA |
| | | GAACGTTAGT GGACAGAGGC TGGGGAAATG GATGTGGACT TTTTGGCAAA |
| | | GGGAGCCTGG TGACATGCGC TAAGTTTGCA TGCTCCAAGA AAATGACCGG |
| | | GAAGAGCATC CAGCCAGAGA ATCTGGAGTA CCGATAATGC TGTCAGTTCA |
| | | TGGCTCCCAG CACAGTGGGA TGATCGTTAA TGACACAGGA CATGAAACTG |
| | | ATGAGAATAG AGCGAAAGTT GAGATAACGC CCAATTCACC GAGAGCCGAA |
| | | GCCACCCTGG GGGGTTTTGG AAGCCTAGGA CTTGATTGTG AACCGAGGAC |
| | | AGGCCTTGAC TTTTCAGATT TGTATTACTT GACTATGAAT AACAAGCACT |
| | | GGTTGGTTCA CAAGGAGTGG TTCCACGACA TTCCATTACC TTGGCACGCT |
| | | GGGGCAGACA CCGGAACTCC ACACTGGAAC AACAAAGAAG CACTGGTAGA |
| | | GTTCAAGGAC GCACATGCCA AAAGGCAAAC TGTCGTGGTT CTAGGGAGTC |
| | | AAGAAGGAGC AGTTCACACG GCCCTTGCTG GAGCTCTGGA GGCTGAGATG |
| | | GATGGTGCAA AGGGAAGGCT GTCCTCTGGC CACCTGAAAT GTCGCCTGAA |
| | | AATGGATAAA CTTAGATTGA AGGGCGTGTC ATACTCCTTG TGTACTGCAG |
| | | CGTTCACATT CACCAAGATC CCGGCTGAAA CACTGCACGG GACAGTCACA |
| | | GTGGAGTTAC AGTACGCAGG GACAGATGGA CCTTGCAAGG TTCCAGCTCA |
| | | GATGGCGGTG GACATGCAAA CTCTGACCCC AGTTGGGAGG TTGATAACCG |
| | | CTAACCCCGT AATCACTGAA AGCACTGAGA ACTCTAAGAT GATGCTGGAA |
| | | CTTGATCCAC CATTTGGGGA CTCTTACATT GTCATAGGAG TCGGGGAGAA |
| | | GAAGATCACC CACCACTGGC ACAGGAGTGG CAGCACCATT GGAAAAGCAT |
| | | TTGAAGCCAC TGTGAGAGGT GCCAAGAGAA TGGCAGTCTT GGGAGACACA |
| | | GCCTGGGACT TTGGATCAGT TGGAGGCGCT CTCAACTCAT TGGGCAAGGG |
| | | CATCCATCAA ATTTTTGGAG CAGCTTTCAA ATCATTGTTT GGAGGAATGT |
| | | CCTGGTTCTC ACAAATTCTC ATTGGAACGT TGCTGATGTG GTTGGGTCTG |
| | | AACACAAAGA ATGGATCTAT TTCCCTTATG TGCTTGGCCT TAGGGGGAGT |
| | | GTTGATCTTC TTATCCACAG CCGTCTCTGC T |
| 35 | ZV CME | MKNPKKKSGG FRIVNMLKRG VARVSPFGGL KRLPAGLLLG HGPIRMVLAI |
| | AA | LAFLRFTAIK PSLGLINRWG SVGKKEAMET IKKEKKDLAA MLRIINARKE |
| | | KKRRGADTSV GIVGLLLTTT AMAAEVTRRG SAYYMYLDRN DAGEAISEPT |
| | | TLGMNKCYIQ IMDLGHMCDA TMSYECPMLD EGVEPDDVDC WCNTTSTWVV |
| | | YGTCHHKKGE ARRSRRAVTL PSHSTRKLQT RSQTWLESRE YTKHLIRVEN |
| | | WIFRNPGFAL AAAAIAWLLG SSTSQKVIYL VMILLIAPAY SIRCIGVSNR |
| | | DFVEGMSGGT WVDVVLEHGG CVTVMAQDKP TVDIELVTTT VSNMAEVRSY |
| | | CYEASISDMA SDSRCPTQGE AYLDKQSDTQ YVCKRTLVDR GWGNGCGLFG |
| | | KGSLVTCAKF ACSKKMTGKS IQPENLEYRI MLSVHGSQHS GMIVNDTGHE |
| | | TDENRAKVEI TPNSPRAEAT LGGFGSLGLD CEPRTGLDES DLYYLTMNNK |
| | | HWLVHKEWFH DIPLPWHAGA DTGTPHWNNK EALVEFKDAH AKRQTVVVLG |
| | | SQEGAVHTAL AGALEAEMDG AKGRLSSGHL KCRLKMDKLR LKGVSYSLCT |
| | | AAFTFTKIPA ETLHGTVTVE LQYAGTDGPC KVPAQMAVDM QTLTPVGRLI |
| | | TANPVITEST ENSKMMLELD PPFGDSYIVI GVGEKKITHH WHRSGSTIGK |
| | | AFEATVRGAK RMAVLGDTAW DFGSVGGALN SLGKGIHQIF GAAFKSLEGG |
| | | MSWFSQILIG TLLMWLGLNT KNGSISLMCL ALGGVLIELS TAVSA |
| 36 | ZV C | MKNPKKKSGG FRIVNMLKRG VARVSPFGGL KRLPAGLLLG HGPIRMVLAI |
| | AA | LAFLRFTAIK PSLGLINRWG SVGKKEAMET IKKFKKDLAA MLRIINARKE |
| | | KKRRGADTSV GIVGLLLTT |
| 37 | ZV prM | AMAAEVTRRG SAYYMYLDRN DAGEAISFPT TLGMNKCYIQ IMDLGHMCDA |
| | AA | TMSYECPMLD EGVEPDDVDC WCNTTSTWVV YGTCHHKKGE ARRSRR |
| 38 | ZVE AA | AVTLPSHSTR KLQTRSQTWL ESREYTKHLI RVENWIERNP GFALAAAAIA |
| | | WLLGSSTSQK VIYLVMILLI APAYSIRCIG VSNRDEVEGM SGGTWVDVVL |
| | | EHGGCVTVMA QDKPTVDIEL VTTTVSNMAE VRSYCYEASI SDMASDSRCP |
| | | TQGEAYLDKQ SDTQYVCKRT LVDRGWGNGC GLFGKGSLVT CAKFACSKKM |
| | | TGKSIQPENL EYRIMLSVHG SQHSGMIVND TGHETDENRA KVEITPNSPR |
| | | AEATLGGFGS LGLDCEPRTG LDFSDLYYLT MNNKHWLVHK EWFHDIPLPW |
| | | HAGADTGTPH WNNKEALVEF KDAHAKRQTV VVLGSQEGAV HTALAGALEA |
| | | EMDGAKGRLS SGHLKCRLKM DKLRLKGVSY SLCTAAFTFT KIPAETLHGT |
| | | VTVELQYAGT DGPCKVPAQM AVDMQTLTPV GRLITANPVI TESTENSKMM |
| | | LELDPPFGDS YIVIGVGEKK ITHHWHRSGS TIGKAFEATV RGAKRMAVLG |
| | | DTAWDFGSVG GALNSLGKGI HQIFGAAFKS LFGGMSWFSQ ILIGTLLMWL |
| | | GLNTKNGSIS LMCLALGGVL IFLSTAVSA |

What is claimed is:

1. A viral vector system, comprising:
one or more viral vector types, each viral vector type comprising:
an outer layer comprising structural proteins of a non-dengue Orthoflavivirus species; and
a nucleic acid for expressing one or more nonstructural proteins of dengue virus.

2. The viral vector system of claim 1, wherein the non-dengue Orthoflavivirus species is Japanese encephalitis virus, Tick-borne encephalitis virus, West Nile virus, Powassan virus, Yellow fever virus, or Zika virus.

3. The viral vector system of claim 1, wherein the structural proteins comprise one or more of: C protein, E protein, and M protein.

4. The viral vector system of claim 1, wherein the one or more nonstructural proteins comprise one or more of: NS1, NS2A, NS2B, NS3, NS4A, NS4B, and NS5.

5. The viral vector system of claim 1, wherein the nucleic acid comprises a sequence encoding the one or more nonstructural proteins of dengue virus that is at least 70% identical to its native sequence.

6. The viral vector system of claim 1, comprising two or more serotypes of dengue virus to yield a multivalent viral vector system.

7. The viral vector system of any one of claim 6, wherein the viral vector system comprises two or more subtypes of one or more genotypes of dengue virus.

8. The viral vector system of any one of claims 1-7, wherein the viral vector system is formulated for vaccine administration.

9. A method of yielding viral vector products, comprising:
contacting a culture of packaging cells with:
a nucleic acid comprising a first set of one or more expression constructs that encodes for one or more nonstructural proteins of dengue virus with a 5′-UTR a 3′-UTR, and a packaging signal, wherein the one or more nonstructural proteins are derived from native sequences of dengue virus; and
a nucleic acid comprising a second set of one or more expression constructs that encodes for one or more structural proteins of a non-dengue Orthoflavivirus species, wherein the one or more structural proteins are derived from a native sequence of non-dengue Orthoflavivirus species.

10. The method of claim 9 further comprising:
preparing the viral vector products for vaccine administration.

11. The method of claim 9, wherein the non-dengue Orthoflavivirus species is Japanese encephalitis virus, Tick-borne encephalitis virus, West Nile virus, Powassan virus, Yellow fever virus, or Zika virus.

12. The method of claim 9, wherein the structural proteins comprise one or more of: C protein, E protein, and M protein, or a precursor protein thereof.

13. The method of claim 9, wherein the one or more nonstructural proteins comprise one or more of: NS1, NS2A, NS2B, NS3, NS4A, NS4B, and NS5.

14. The method of claim 9, wherein the viral vector products comprise:
an outer layer comprising the one or more structural proteins of the non-dengue Orthoflavivirus species nucleic acid; and
a positive-strand RNA comprising sequences of the 5′-UTR, the 3′-UTR, packaging signal, and a genetic code for the one or more nonstructural proteins of the dengue virus.

15. A viral vector system, comprising:
one or more viral vector types, each viral vector type comprising:
an outer layer comprising structural proteins of Zika virus; and
a nucleic acid for expressing one or more nonstructural proteins of a non-Zika Orthoflavivirus species.

16. The viral vector system of claim 15, wherein the non-Zika Orthoflavivirus species is Japanese encephalitis virus, Tick-borne encephalitis virus, West Nile virus, Powassan virus, Yellow fever virus, or dengue virus.

17. The viral vector system of claim 15, wherein the structural proteins comprise one or more of: C protein, E protein, and M protein.

18. The viral vector system of claim 15, wherein the one or more nonstructural proteins comprise one or more of: NS1, NS2A, NS2B, NS3, NS4A, NS4B, and NS5.

19. The viral vector system of claim 15, wherein the nucleic acid comprises a sequence encoding the one or more nonstructural proteins of the non-Zika Orthoflavivirus species that is at least 70% identical to its native sequence.

20. The viral vector system of claim 15, wherein the viral vector system comprises a multivalent viral vector system comprises two or more serotypes of the non-Zika Orthoflavivirus species.

21. The viral vector system of claim 15, wherein the viral vector system is formulated for vaccine administration.
